SEQUENCE LISTING (368 sequences)

SEQ ID NO: 1 - protein, 25 aa, synthetic construct
EVQLVESGGDLVKPGGSLRLSCVAS

SEQ ID NO: 2 - protein, 12 aa, synthetic construct
WVRQAPGKGLQW

SEQ ID NO: 3 - protein, 35 aa, synthetic construct
DAVKGRFTISRDNAKNTLYLQMNSLRAEDTAVYYC

SEQ ID NO: 4 - protein, 11 aa, synthetic construct
WGQGTLVTVSS

SEQ ID NO: 5 - protein, 25 aa, synthetic construct
QSVLTQPTSVSGSLGQRVTISCSGS

SEQ ID NO: 6 - protein, 14 aa, synthetic construct
WYQQLPGKAPKLLV

SEQ ID NO: 7 - protein, 32 aa, synthetic construct
GVPDRFSGSKSGNSATLTITGLQAEDEADYYC

SEQ ID NO: 8 - protein, 10 aa, synthetic construct
FGGGTHLTVL

SEQ ID NO: 9 - protein, 25 aa, synthetic construct
QAVLTQPPSVSAALGQRVTISCTGS

SEQ ID NO: 10 - protein, 14 aa, synthetic construct
WYQQLPGKSPKTII

SEQ ID NO: 11 - protein, 32 aa, synthetic construct
GVPVRFSGSKSGSTATLTITGIQAEDEADYYC

SEQ ID NO: 12 - protein, 25 aa, synthetic construct
ELTLQESGPGLVKPSQTLSLTCVVS

SEQ ID NO: 13 - protein, 12 aa, synthetic construct
WIRQRPGRGLEW

SEQ ID NO: 14 - protein, 35 aa, synthetic construct
PAFQGRISITADTAKNQFSLQLSSMTTEDTAVYYC

SEQ ID NO: 15 - protein, 25 aa, synthetic construct
QSMLTQPASVSGSLGQKVTISCTGS

SEQ ID NO: 16 - protein, 14 aa, synthetic construct
WYQQLPGIGPRTVI

SEQ ID NO: 17 - protein, 32 aa, synthetic construct
GVPDRFSGSKSGSSATLTISGLQAEDEAEYYC

SEQ ID NO: 18 - protein, 10 aa, synthetic construct
GFTFSNYGMT

SEQ ID NO: 19 - protein, 10 aa, synthetic construct
GFTFSSFGMN

SEQ ID NO: 20 - protein, 10 aa, synthetic construct
GFTFSNYGMN

SEQ ID NO: 21 - protein, 10 aa, synthetic construct
GFSFSDSGLN

SEQ ID NO: 22 - protein, 10 aa, synthetic construct
GFTFSNYNMG

SEQ ID NO: 23 - protein, 10 aa, synthetic construct
GFTFNNYGMS

SEQ ID NO: 24 - protein, 10 aa, synthetic construct
GFTFSTYGMT

SEQ ID NO: 25 - protein, 10 aa, synthetic construct
GFTFSDYNMG

SEQ ID NO: 26 - protein, 10 aa, synthetic construct
GFTFIRFGMS

SEQ ID NO: 27 - protein, 10 aa, synthetic construct
GFTFSDYNMA

SEQ ID NO: 28 - protein, 10 aa, synthetic construct
GFTFSTYGMN

SEQ ID NO: 29 - protein, 10 aa, synthetic construct
GFTFSDYGMN

SEQ ID NO: 30 - protein, 10 aa, synthetic construct
GFTFSTYNMG

SEQ ID NO: 31 - protein, 10 aa, synthetic construct
GFTFSDYGMT

SEQ ID NO: 32 - protein, 10 aa, synthetic construct
GFTFSNYGLS

SEQ ID NO: 33 - protein, 10 aa, synthetic construct
GFTFSNSGMS

SEQ ID NO: 34 - protein, 10 aa, synthetic construct
GFTFSNYPMT

SEQ ID NO: 35 - protein, 10 aa, synthetic construct
GFTFRSYGMG

SEQ ID NO: 36 - protein, 10 aa, synthetic construct
GFTFNIYGMS

SEQ ID NO: 37 - protein, 10 aa, synthetic construct
GFTFSRYNMG

SEQ ID NO: 38 - protein, 11 aa, synthetic construct
GGSVTSSYYWN

SEQ ID NO: 39 - protein, 11 aa, synthetic construct
GGSVTSNYYWN

SEQ ID NO: 40 - protein, 11 aa, synthetic construct
GGSVTSTYYWN

SEQ ID NO: 41 - protein, 11 aa, synthetic construct
RGSVTSSYYWN

SEQ ID NO: 42 - protein, 11 aa, synthetic construct
GGSVTNSYYWN

SEQ ID NO: 43 - protein, 11 aa, synthetic construct
GGSVTSDYYWN

SEQ ID NO: 44 - protein, 11 aa, synthetic construct
GGSVTSSHYWN

SEQ ID NO: 45 - protein, 11 aa, synthetic construct
GGSVTSSSYWT

SEQ ID NO: 46 - protein, 11 aa, synthetic construct
GGSVTSSSYWN

SEQ ID NO: 47 - protein, 11 aa, synthetic construct
GGSVSSSYYWN

SEQ ID NO: 48 - protein, 11 aa, synthetic construct
GGSVTSGYYWN

SEQ ID NO: 49 - protein, 11 aa, synthetic construct
GGSVTTSYYWN

SEQ ID NO: 50 - protein, 11 aa, synthetic construct
GGSVTNNYYWN

SEQ ID NO: 51 - protein, 11 aa, synthetic construct
GGSVTNTYYWN

SEQ ID NO: 52 - protein, 11 aa, synthetic construct
GGSVTTTYYWN

SEQ ID NO: 53 - protein, 11 aa, synthetic construct
GGSVTSSYYWS

SEQ ID NO: 54 - protein, 11 aa, synthetic construct
GDSVTSSYYWN

SEQ ID NO: 55 - protein, 11 aa, synthetic construct
GDSVTSNYYWN

SEQ ID NO: 56 - protein, 11 aa, synthetic construct
GGSVTSSYHWN

SEQ ID NO: 57 - protein, 11 aa, synthetic construct
GGSVTSSYYWH

SEQ ID NO: 58 - protein, 14 aa, synthetic construct
VANIDSSGTETTYV

SEQ ID NO: 59 - protein, 14 aa, synthetic construct
VAWIYASGSSTSYA

SEQ ID NO: 60 - protein, 14 aa, synthetic construct
VAYIKKGGSWTTYA

SEQ ID NO: 61 - protein, 14 aa, synthetic construct
VTFINSGGSRTTYA

SEQ ID NO: 62 - protein, 14 aa, synthetic construct
VASINSAGSGTGYA

SEQ ID NO: 63 - protein, 14 aa, synthetic construct
VAYINSPGNSVDYP

SEQ ID NO: 64 - protein, 14 aa, synthetic construct
VAYINSGGTNTDYA

SEQ ID NO: 65 - protein, 14 aa, synthetic construct
VAHIDSGGINTNFA

SEQ ID NO: 66 - protein, 14 aa, synthetic construct
VAYIDSGGSSTTYS

SEQ ID NO: 67 - protein, 14 aa, synthetic construct
VAYIDSSGSDTLYA

SEQ ID NO: 68 - protein, 14 aa, synthetic construct
VANINAGGTSTGYI

SEQ ID NO: 69 - protein, 14 aa, synthetic construct
VAYIKPGQTFITYL

SEQ ID NO: 70 - protein, 14 aa, synthetic construct
VAYINSGGTTTHYA

SEQ ID NO: 71 - protein, 14 aa, synthetic construct
VAYIHSGGTTSTYG

SEQ ID NO: 72 - protein, 14 aa, synthetic construct
VAYINSGATTTHYA

SEQ ID NO: 73 - protein, 14 aa, synthetic construct
VAWIYGSGTGTSYG

SEQ ID NO: 74 - protein, 14 aa, synthetic construct
VAYIDRSGSHTIYA

SEQ ID NO: 75 - protein, 14 aa, synthetic construct
VAQINIGGSITHYA

SEQ ID NO: 76 - protein, 14 aa, synthetic construct
VADIDKGGSSTRYA

SEQ ID NO: 77 - protein, 14 aa, synthetic construct
VAYINSGGNTIGYA

SEQ ID NO: 78 - protein, 11 aa, synthetic construct
MGYWTGSTNYN

SEQ ID NO: 79 - protein, 11 aa, synthetic construct
MGYWTGTTNYN

SEQ ID NO: 80 - protein, 11 aa, synthetic construct
MGYWTGNTNYN

SEQ ID NO: 81 - protein, 11 aa, synthetic construct
MGYWTGSTKYN

SEQ ID NO: 82 - protein, 11 aa, synthetic construct
MGYWIGSTSYN

SEQ ID NO: 83 - protein, 11 aa, synthetic construct
MGYWTGSTSYN

SEQ ID NO: 84 - protein, 11 aa, synthetic construct
MGYWAGSTNYN

SEQ ID NO: 85 - protein, 11 aa, synthetic construct
MGYWTGRTNYN

SEQ ID NO: 86 - protein, 11 aa, synthetic construct
MGYWTGTTKYN

SEQ ID NO: 87 - protein, 11 aa, synthetic construct
MGYWTGDTNYN

SEQ ID NO: 88 - protein, 11 aa, synthetic construct
MGYWTGNTKYN

SEQ ID NO: 89 - protein, 11 aa, synthetic construct
MGYWTGGTNYN

SEQ ID NO: 90 - protein, 11 aa, synthetic construct
MGYWVGSTTYN

SEQ ID NO: 91 - protein, 11 aa, synthetic construct
MGYWTGTTRYN

SEQ ID NO: 92 - protein, 11 aa, synthetic construct
LGYWTGSTNYN

SEQ ID NO: 93 - protein, 11 aa, synthetic construct
MGYWTGSTDYN

SEQ ID NO: 94 - protein, 11 aa, synthetic construct
MGFWTGSTNYN

SEQ ID NO: 95 - protein, 11 aa, synthetic construct
MGYWSGSTNYN

SEQ ID NO: 96 - protein, 11 aa, synthetic construct
MGYWTGYTNYN

SEQ ID NO: 97 - protein, 11 aa, synthetic construct
MGYWTGSTTYN

SEQ ID NO: 98 - protein, 16 aa, synthetic construct
TSTPIYTDAYVANFDY

SEQ ID NO: 99 - protein, 16 aa, synthetic construct
ASDGDTFGYILNGFHS

SEQ ID NO: 100 - protein, 16 aa, synthetic construct
AKEVRDTHGLTFNFDS

SEQ ID NO: 101 - protein, 16 aa, synthetic construct
AWSDYNSYRLGDYLDH

SEQ ID NO: 102 - protein, 16 aa, synthetic construct
ASESARVIHAYVWFYN

SEQ ID NO: 103 - protein, 16 aa, synthetic construct
ARSPLGYYGSFSNFDY

SEQ ID NO: 104 - protein, 16 aa, synthetic construct
ATVWAPYGYGSHNFDY

SEQ ID NO: 105 - protein, 16 aa, synthetic construct
ARSPLGYYGSVSNFDY

SEQ ID NO: 106 - protein, 16 aa, synthetic construct
ARSPPGYYGSVSNFDY

SEQ ID NO: 107 - protein, 16 aa, synthetic construct
ANYEGRYWETVYNFQY

SEQ ID NO: 108 - protein, 16 aa, synthetic construct
AKDGYDGDIWIDNFDY

SEQ ID NO: 109 - protein, 16 aa, synthetic construct
AKDGEVKNNWYNWFQY

SEQ ID NO: 110 - protein, 16 aa, synthetic construct
ANEGPSYYQGLNWFYY

SEQ ID NO: 111 - protein, 16 aa, synthetic construct
VRLNLAGWGLLREDNF

SEQ ID NO: 112 - protein, 16 aa, synthetic construct
ASGIYIWIRSHRNFEY

SEQ ID NO: 113 - protein, 16 aa, synthetic construct
ATPGLGDNKYWEEVIY

SEQ ID NO: 114 - protein, 16 aa, synthetic construct
AKAADINLWYGRGFDQ

SEQ ID NO: 115 - protein, 16 aa, synthetic construct
AGLDGGRFSGGNAFGY

SEQ ID NO: 116 - protein, 16 aa, synthetic construct
ATTTGYGTYETYNIDY

SEQ ID NO: 117 - protein, 16 aa, synthetic construct
TTSGRTPSGAKYNFAH

SEQ ID NO: 118 - protein, 15 aa, synthetic construct
ASLPGSSWSGYNFDY

SEQ ID NO: 119 - protein, 19 aa, synthetic construct
ARDGGWRYGWLGVARPFDY

SEQ ID NO: 120 - protein, 15 aa, synthetic construct
VRNRYYGSSSWDFDY

SEQ ID NO: 121 - protein, 16 aa, synthetic construct
ASLRRYFGSYYSESDL

SEQ ID NO: 122 - protein, 13 aa, synthetic construct
ASLPGSSWSHFDY

SEQ ID NO: 123 - protein, 11 aa, synthetic construct
ARGYDSYHPRY

SEQ ID NO: 124 - protein, 15 aa, synthetic construct
ARRGNIWANGGLFDY

SEQ ID NO: 125 - protein, 16 aa, synthetic construct
ARDGSRGIYGSGNFHY

SEQ ID NO: 126 - protein, 15 aa, synthetic construct
VRRSDIWSSNGHFDY

SEQ ID NO: 127 - protein, 13 aa, synthetic construct
ARVAIAPWNGFDY

SEQ ID NO: 128 - protein, 15 aa, synthetic construct
AKAYYGGYDGYNLEY

SEQ ID NO: 129 - protein, 12 aa, synthetic construct
ARRGPHFGSYYY

SEQ ID NO: 130 - protein, 14 aa, synthetic construct
ARRGSSWYYTYFDY

SEQ ID NO: 131 - protein, 11 aa, synthetic construct
ARYIYGSGFDY

SEQ ID NO: 132 - protein, 15 aa, synthetic construct
ASLPGSSWSGYNFEY

SEQ ID NO: 133 - protein, 13 aa, synthetic construct
ASTGSIYGYAPFN

SEQ ID NO: 134 - protein, 10 aa, synthetic construct
ARGSSGNFDY

SEQ ID NO: 135 - protein, 15 aa, synthetic construct
ASGSDIWSSNGHFDS

SEQ ID NO: 136 - protein, 17 aa, synthetic construct
ARALEGSSWYSTVNFDY

SEQ ID NO: 137 - protein, 18 aa, synthetic construct
ARGRRGYIYGYADGHFDF

SEQ ID NO: 138 - protein, 10 aa, synthetic construct
TNNIGIVGAS

SEQ ID NO: 139 - protein, 10 aa, synthetic construct
TNNIGIVGAN

SEQ ID NO: 140 - protein, 10 aa, synthetic construct
TNNIGLVGAS

SEQ ID NO: 141 - protein, 10 aa, synthetic construct
TNNIGIVGAT

SEQ ID NO: 142 - protein, 10 aa, synthetic construct
TGNIGVVGAT

SEQ ID NO: 143 - protein, 10 aa, synthetic construct
TNNIGIAGAS

SEQ ID NO: 144 - protein, 10 aa, synthetic construct
TDNIGILGAT

SEQ ID NO: 145 - protein, 10 aa, synthetic construct
TNNIGTVGAS

SEQ ID NO: 146 - protein, 10 aa, synthetic construct
TNNIGVVGAS

SEQ ID NO: 147 - protein, 10 aa, synthetic construct
TNNIGIVGAA

SEQ ID NO: 148 - protein, 10 aa, synthetic construct
TNNIGTVGAT

SEQ ID NO: 149 - protein, 10 aa, synthetic construct
ANNIGIFGAA

SEQ ID NO: 150 - protein, 10 aa, synthetic construct
TDDIGLIGAT

SEQ ID NO: 151 - protein, 10 aa, synthetic construct
TNNIGILGAT

SEQ ID NO: 152 - protein, 10 aa, synthetic construct
TDNIGTIGAR

SEQ ID NO: 153 - protein, 10 aa, synthetic construct
ANNIGIVGAT

SEQ ID NO: 154 - protein, 10 aa, synthetic construct
TNDIRIVGAS

SEQ ID NO: 155 - protein, 10 aa, synthetic construct
TNNINIVGAS

SEQ ID NO: 156 - protein, 10 aa, synthetic construct
TNDIGIVGAT

SEQ ID NO: 157 - protein, 10 aa, synthetic construct
TDNVGIVGAN

SEQ ID NO: 158 - protein, 11 aa, synthetic construct
NTNIGSGYDVQ

SEQ ID NO: 159 - protein, 11 aa, synthetic construct
DTNIGSGYDVQ

SEQ ID NO: 160 - protein, 11 aa, synthetic construct
NTNIGSAYDVQ

SEQ ID NO: 161 - protein, 11 aa, synthetic construct
NTNIGRGYDVQ

SEQ ID NO: 162 - protein, 11 aa, synthetic construct
NTNIGRGHDVQ

SEQ ID NO: 163 - protein, 11 aa, synthetic construct
NTNIGGGYDVQ

SEQ ID NO: 164 - protein, 11 aa, synthetic construct
NTNIGSGYGVQ

SEQ ID NO: 165 - protein, 11 aa, synthetic construct
NTNIGSNYDVQ

SEQ ID NO: 166 - protein, 11 aa, synthetic construct
NTNIGSDYDVQ

SEQ ID NO: 167 - protein, 11 aa, synthetic construct
NTNIGKDYDVQ

SEQ ID NO: 168 - protein, 11 aa, synthetic construct
NTNIGSVYDVQ

SEQ ID NO: 169 - protein, 11 aa, synthetic construct
NTNIGSGYDVH

SEQ ID NO: 170 - protein, 11 aa, synthetic construct
NTNIGSGFDVQ

SEQ ID NO: 171 - protein, 11 aa, synthetic construct
DTNIGGDYDVQ

SEQ ID NO: 172 - protein, 11 aa, synthetic construct
DTNIGSNYDVQ

SEQ ID NO: 173 - protein, 11 aa, synthetic construct
DTNIGTGYDVQ

SEQ ID NO: 174 - protein, 11 aa, synthetic construct
NTNIGKNYDVH

SEQ ID NO: 175 - protein, 11 aa, synthetic construct
ETNIGKNYDVQ

SEQ ID NO: 176 - protein, 11 aa, synthetic construct
NTNIGSYFDVQ

SEQ ID NO: 177 - protein, 11 aa, synthetic construct
HTNFGSGYVAQ

SEQ ID NO: 178 - protein, 10 aa, synthetic construct
SSNIGGNYVG

SEQ ID NO: 179 - protein, 10 aa, synthetic construct
SSNIGGNYVA

SEQ ID NO: 180 - protein, 10 aa, synthetic construct
SSNIGDNYVG

SEQ ID NO: 181 - protein, 10 aa, synthetic construct
SSNIGGNNVG

SEQ ID NO: 182 - protein, 10 aa, synthetic construct
NSNIGGNYVG

SEQ ID NO: 183 - protein, 10 aa, synthetic construct
RSNIGGNYVG

SEQ ID NO: 184 - protein, 10 aa, synthetic construct
SSNIGGNFVG

SEQ ID NO: 185 - protein, 10 aa, synthetic construct
SSNIGDNYVA

SEQ ID NO: 186 - protein, 10 aa, synthetic construct
SSNIGANYVA

SEQ ID NO: 187 - protein, 10 aa, synthetic construct
SSNIGGNSVG

SEQ ID NO: 188 - protein, 10 aa, synthetic construct
NSNIGGNYVA

SEQ ID NO: 189 - protein, 10 aa, synthetic construct
SSNIGNNYVG

SEQ ID NO: 190 - protein, 10 aa, synthetic construct
GSNIRTNYVG

SEQ ID NO: 191 - protein, 10 aa, synthetic construct
SSNIGGYYVG

SEQ ID NO: 192 - protein, 10 aa, synthetic construct
SSNIGGNHVG

SEQ ID NO: 193 - protein, 10 aa, synthetic construct
SSNIGANYVG

SEQ ID NO: 194 - protein, 10 aa, synthetic construct
SSNIGGNDVG

SEQ ID NO: 195 - protein, 10 aa, synthetic construct
SSNIGSNYVG

SEQ ID NO: 196 - protein, 10 aa, synthetic construct
SSNIGTNYVG

SEQ ID NO: 197 - protein, 10 aa, synthetic construct
GSNIGGNYVG

SEQ ID NO: 198 - protein, 8 aa, synthetic construct
DSDGDRPS

SEQ ID NO: 199 - protein, 8 aa, synthetic construct
YSDGDRPS

SEQ ID NO: 200 - protein, 8 aa, synthetic construct
YSDGNRPS

SEQ ID NO: 201 - protein, 8 aa, synthetic construct
YSDGSRPS

SEQ ID NO: 202 - protein, 8 aa, synthetic construct
YNDGNRPS

SEQ ID NO: 203 - protein, 8 aa, synthetic construct
DNDGDRPS

SEQ ID NO: 204 - protein, 8 aa, synthetic construct
YSNGDRPS

SEQ ID NO: 205 - protein, 8 aa, synthetic construct
YDDGDRPS

SEQ ID NO: 206 - protein, 8 aa, synthetic construct
YTNGDRPS

SEQ ID NO: 207 - protein, 8 aa, synthetic construct
YSDGKRPS

SEQ ID NO: 208 - protein, 8 aa, synthetic construct
HSDGDRPS

SEQ ID NO: 209 - protein, 8 aa, synthetic construct
DIDGDRPS

SEQ ID NO: 210 - protein, 8 aa, synthetic construct
YRNGDRPS

SEQ ID NO: 211 - protein, 8 aa, synthetic construct
FSDGDRPS

SEQ ID NO: 212 - protein, 8 aa, synthetic construct
HSDGERPS

SEQ ID NO: 213 - protein, 8 aa, synthetic construct
DSDGNRPS

SEQ ID NO: 214 - protein, 8 aa, synthetic construct
DSDGERPS

SEQ ID NO: 215 - protein, 8 aa, synthetic construct
YNNGDRPS

SEQ ID NO: 216 - protein, 8 aa, synthetic construct
DGDGDRPS

SEQ ID NO: 217 - protein, 8 aa, synthetic construct
YTTGDRPS

SEQ ID NO: 218 - protein, 8 aa, synthetic construct
YGNSNRPS

SEQ ID NO: 219 - protein, 8 aa, synthetic construct
YGNTNRPS

SEQ ID NO: 220 - protein, 8 aa, synthetic construct
YGNNNRPS

SEQ ID NO: 221 - protein, 8 aa, synthetic construct
YGDSNRPS

SEQ ID NO: 222 - protein, 8 aa, synthetic construct
YGNRNRPS

SEQ ID NO: 223 - protein, 8 aa, synthetic construct
YGNINRPS

SEQ ID NO: 224 - protein, 8 aa, synthetic construct
YGYTNRPS

SEQ ID NO: 225 - protein, 8 aa, synthetic construct
YGNSYRPS

SEQ ID NO: 226 - protein, 8 aa, synthetic construct
YGNGNRPS

SEQ ID NO: 227 - protein, 8 aa, synthetic construct
FGNSNRPS

SEQ ID NO: 228 - protein, 8 aa, synthetic construct
YGNSDRPS

SEQ ID NO: 229 - protein, 8 aa, synthetic construct
FGNTNRPS

SEQ ID NO: 230 - protein, 8 aa, synthetic construct
YGTTFRPS

SEQ ID NO: 231 - protein, 8 aa, synthetic construct
YANSNRPS

SEQ ID NO: 232 - protein, 8 aa, synthetic construct
HGDSNRPS

SEQ ID NO: 233 - protein, 8 aa, synthetic construct
YGDNNRPS

SEQ ID NO: 234 - protein, 8 aa, synthetic construct
YGDTNRPS

SEQ ID NO: 235 - protein, 8 aa, synthetic construct
YDNTNRPS

SEQ ID NO: 236 - protein, 8 aa, synthetic construct
YGNDNRPS

SEQ ID NO: 237 - protein, 8 aa, synthetic construct
YGTRNRPS

SEQ ID NO: 238 - protein, 8 aa, synthetic construct
YGNNYRPS

SEQ ID NO: 239 - protein, 8 aa, synthetic construct
YGNDYRPS

SEQ ID NO: 240 - protein, 8 aa, synthetic construct
YGDNYRPS

SEQ ID NO: 241 - protein, 8 aa, synthetic construct
YANNYRPS

SEQ ID NO: 242 - protein, 8 aa, synthetic construct
YGNGFRPP

SEQ ID NO: 243 - protein, 8 aa, synthetic construct
YGNDHRPS

SEQ ID NO: 244 - protein, 8 aa, synthetic construct
YSNNYRPS

SEQ ID NO: 245 - protein, 8 aa, synthetic construct
YGDDYRPS

SEQ ID NO: 246 - protein, 8 aa, synthetic construct
YGNSYRPS

SEQ ID NO: 247 - protein, 8 aa, synthetic construct
YGNNHRPS

SEQ ID NO: 248 - protein, 8 aa, synthetic construct
YGNYYRPS

SEQ ID NO: 249 - protein, 8 aa, synthetic construct
YRDTYRPS

SEQ ID NO: 250 - protein, 8 aa, synthetic construct
YGNNNRPS

SEQ ID NO: 251 - protein, 8 aa, synthetic construct
YGDDHRPS

SEQ ID NO: 252 - protein, 8 aa, synthetic construct
YGNSHRPS

SEQ ID NO: 253 - protein, 8 aa, synthetic construct
YGDNHRPS

SEQ ID NO: 254 - protein, 8 aa, synthetic construct
YGNTYRPS

SEQ ID NO: 255 - protein, 8 aa, synthetic construct
YGYNYRPS

SEQ ID NO: 256 - protein, 8 aa, synthetic construct
FGNNYRPS

SEQ ID NO: 257 - protein, 8 aa, synthetic construct
YDNNYRPS

SEQ ID NO: 258 - protein, 11 aa, synthetic construct
QSFDTTLDAAV

SEQ ID NO: 259 - protein, 11 aa, synthetic construct
QSFDTTLDAYV

SEQ ID NO: 260 - protein, 11 aa, synthetic construct
QSFDTTLDAGV

SEQ ID NO: 261 - protein, 12 aa, synthetic construct
QSFDTTLDAYAV

SEQ ID NO: 262 - protein, 11 aa, synthetic construct
QSFDTTLDAHV

SEQ ID NO: 263 - protein, 12 aa, synthetic construct
QSFDTTLDAHAV

SEQ ID NO: 264 - protein, 10 aa, synthetic construct
QSFDTTLDAV

SEQ ID NO: 265 - protein, 11 aa, synthetic construct
QSFDTTLDAPV

SEQ ID NO: 266 - protein, 11 aa, synthetic construct
QSFDIALQAHV

SEQ ID NO: 267 - protein, 11 aa, synthetic construct
QSFDTTLDASV

SEQ ID NO: 268 - protein, 11 aa, synthetic construct
QSFDTTLDAIV

SEQ ID NO: 269 - protein, 11 aa, synthetic construct
QSFDTTLDGAV

SEQ ID NO: 270 - protein, 11 aa, synthetic construct
QSFDNTLDIVL

SEQ ID NO: 271 - protein, 11 aa, synthetic construct
QSVDSTLGAAV

SEQ ID NO: 272 - protein, 12 aa, synthetic construct
QSFDTTLDDHVV

SEQ ID NO: 273 - protein, 11 aa, synthetic construct
QSFDATLNAPV

SEQ ID NO: 274 - protein, 10 aa, synthetic construct
QSYETTLDVV

SEQ ID NO: 275 - protein, 9 aa, synthetic construct
QSFDTTLIV

SEQ ID NO: 276 - protein, 12 aa, synthetic construct
QSFDTTLDAHIV

SEQ ID NO: 277 - protein, 11 aa, synthetic construct
QSFDTTLGAAV

SEQ ID NO: 278 - protein, 12 aa, synthetic construct
QSYDDNLDGHAV

SEQ ID NO: 279 - protein, 11 aa, synthetic construct
QSYDDNLDGAV

SEQ ID NO: 280 - protein, 10 aa, synthetic construct
QSYDDNLDAV

SEQ ID NO: 281 - protein, 11 aa, synthetic construct
QSYDDNLDGHV

SEQ ID NO: 282 - protein, 11 aa, synthetic construct
QSYDDNLDGYV

SEQ ID NO: 283 - protein, 11 aa, synthetic construct
QSYDDNLDGIV

SEQ ID NO: 284 - protein, 10 aa, synthetic construct
QSYDDNLDGV

SEQ ID NO: 285 - protein, 12 aa, synthetic construct
QSYDDNLDGHYV

SEQ ID NO: 286 - protein, 12 aa, synthetic construct
QSYDDKFDGHAV

SEQ ID NO: 287 - protein, 11 aa, synthetic construct
QSYDDNFDGAV

SEQ ID NO: 288 - protein, 11 aa, synthetic construct
QSYDDNLDGLV

SEQ ID NO: 289 - protein, 12 aa, synthetic construct
QSYDDNLDGHAA

SEQ ID NO: 290 - protein, 12 aa, synthetic construct
QSYDDNLDGHIV

SEQ ID NO: 291 - protein, 11 aa, synthetic construct
QSYDGDLDGHV

SEQ ID NO: 292 - protein, 11 aa, synthetic construct
QSYDDDLDGAV

SEQ ID NO: 293 - protein, 11 aa, synthetic construct
QSYDDNLGGAV

SEQ ID NO: 294 - protein, 11 aa, synthetic construct
QSYDDNLDDAV

SEQ ID NO: 295 - protein, 10 aa, synthetic construct
QSYDDNLDYV

SEQ ID NO: 296 - protein, 12 aa, synthetic construct
QSYDDNLDGHVV

SEQ ID NO: 297 - protein, 11 aa, synthetic construct
QSYDDNLDGGV

SEQ ID NO: 298 - protein, 11 aa, synthetic construct
SSWDDSLRGHV

SEQ ID NO: 299 - protein, 11 aa, synthetic construct
SSWDDSLRGAV

SEQ ID NO: 300 - protein, 11 aa, synthetic construct
SSWDDSLRGYV

SEQ ID NO: 301 - protein, 11 aa, synthetic construct
SSWDDSLRGIV

SEQ ID NO: 302 - protein, 11 aa, synthetic construct
SSWDDSLRGVV

SEQ ID NO: 303 - protein, 11 aa, synthetic construct
SSWDDSLRGGV

SEQ ID NO: 304 - protein, 11 aa, synthetic construct
SSWDDTLRGHV

SEQ ID NO: 305 - protein, 10 aa, synthetic construct
SSWDDSLQYI

SEQ ID NO: 306 - protein, 11 aa, synthetic construct
SSWDDGLRGHV

SEQ ID NO: 307 - protein, 11 aa, synthetic construct
SSWDDSLRGPV

SEQ ID NO: 308 - protein, 11 aa, synthetic construct
SSWDDSLRGTV

SEQ ID NO: 309 - protein, 11 aa, synthetic construct
SSWDDTLSGHV

SEQ ID NO: 310 - protein, 11 aa, synthetic construct
SSWDNSVRGYV

SEQ ID NO: 311 - protein, 11 aa, synthetic construct
SSWDDSLRGSV

SEQ ID NO: 312 - protein, 11 aa, synthetic construct
SSWDDRLRGAV

SEQ ID NO: 313 - protein, 11 aa, synthetic construct
SSWDDSLRGHL

SEQ ID NO: 314 - protein, 11 aa, synthetic construct
SSWDDSLWTPV

SEQ ID NO: 315 - protein, 11 aa, synthetic construct
SSWDDSVRGVL

SEQ ID NO: 316 - protein, 11 aa, synthetic construct
SSWDDRVRGVL

SEQ ID NO: 317 - protein, 11 aa, synthetic construct
ASWDDNLRVQV

SEQ ID NO: 318 - protein, 159 aa, synthetic construct
  VARIANT 97..102: note = X is one or more naturally-occuring amino acid or absent
  VARIANT 114: note = X is one or more naturally-occuring amino acid or absent
EVQLVESGGDLVKPGGSLRLSCVASGFTFSSYHMSWVRQAPGKGLQWVAYINSGGSSTSYADAVKGRFTISRDNAKNTLYLQMNSLRAEDTAVYYCXXXXXXWGQGTLVTVSSXISONEORMORENATURALLYOCCURINGAMINOACIDORABSENT

SEQ ID NO: 319 - protein, 157 aa, synthetic construct
  VARIANT 95..100: note = X is one or more naturally-occuring amino acid or absent
  VARIANT 112: note = X is one or more naturally-occuring amino acid or absent
ELTLQESGPGLVKPSQTLSLTCVVSGGSVTSSYYWNWIRQRPGRGLEWMGYWTGSTNYNPAFQGRISITADTAKNQFSLQLSSMTTEDTAVYYCXXXXXXWGQGTLVTVSSXISONEORMORENATURALLYOCCURINGAMINOACIDORABSENT

SEQ ID NO: 320 - protein, 152 aa, synthetic construct
  VARIANT 91..96: note = X is one or more naturally-occuring amino acid or absent
  VARIANT 107: note = X is one or more naturally-occuring amino acid or absent
QSVLTQPTSVSGSLGQRVTISCSGSTNNIGIVGASWYQQLPGKAPKLLVDSDGDRPSGVPDRFSGSKSGNSATLTITGLQAEDEADYYCQXXXXXXFGGGTHLTVLXISONEORMORENATURALLYOCCURINGAMINOACIDORABSENT

SEQ ID NO: 321 - protein, 152 aa, synthetic construct
  VARIANT 92..97: note = X is one or more naturally-occuring amino acid or absent
  VARIANT 107: note = X is one or more naturally-occuring amino acid or absent
QAVLTQPPSVSAALGQRVTISCTGSNTNIGSGYDVQWYQQLPGKSPKTIIYGNSNRPSGVPVRFSGSKSGSTATLTITGIQAEDEADYYCQXXXXXXFGGGTHLTVXISONEORMORENATURALLYOCCURINGAMINOACIDORABSENT

SEQ ID NO: 322 - protein, 151 aa, synthetic construct
  VARIANT 91..96: note = X is one or more naturally-occuring amino acid or absent
  VARIANT 106: note = X is one or more naturally-occuring amino acid or absent
QSMLTQPASVSGSLGQKVTISCTGSSSNIGGNYVGWYQQLPGIGPRTVIYGNNYRPSGVPDRFSGSKSGSSATLTISGLQAEDEAEYYCSXXXXXXFGGGTHLTVXISONEORMORENATURALLYOCCURINGAMINOACIDORABSENT

SEQ ID NO: 323 - protein, 4 aa, synthetic construct
GGGS

SEQ ID NO: 324 - protein, 4 aa, synthetic construct
SGGG

SEQ ID NO: 325 - protein, 5 aa, synthetic construct
GGGGS

SEQ ID NO: 326 - protein, 5 aa, synthetic construct
SGGGG

SEQ ID NO: 327 - protein, 6 aa, synthetic construct
GGGGGS

SEQ ID NO: 328 - protein, 6 aa, synthetic construct
SGGGGG

SEQ ID NO: 329 - protein, 7 aa, synthetic construct
GGGGGGS

SEQ ID NO: 330 - protein, 7 aa, synthetic construct
SGGGGGG

SEQ ID NO: 331 - protein, 15 aa, synthetic construct
GGGGSGGGGSGGGGS

SEQ ID NO: 332 - DNA, 41 bp, synthetic construct
  source 1..41: mol_type = other DNA
gaggagcaactggtggagtttggaggacacatggtgaatcc

SEQ ID NO: 333 - DNA, 34 bp, synthetic construct
  source 1..34: mol_type = other DNA
gaactcacactgcaggagtcagggccaggactgg

SEQ ID NO: 334 - DNA, 26 bp, synthetic construct
  source 1..26: mol_type = other DNA
gaggtgcagctggtggagtctggggg

SEQ ID NO: 335 - DNA, 26 bp, synthetic construct
  source 1..26: mol_type = other DNA
gaggtacagctggtggagtctggggg

SEQ ID NO: 336 - DNA, 26 bp, synthetic construct
  source 1..26: mol_type = other DNA
gaggtacagctggtggaatctggggg

SEQ ID NO: 337 - DNA, 26 bp, synthetic construct
  source 1..26: mol_type = other DNA
gaggtgcagctggtggagtctggagg

SEQ ID NO: 338 - DNA, 26 bp, synthetic construct
  source 1..26: mol_type = other DNA
gaggtccagctggtgcagtctggggc

SEQ ID NO: 339 - DNA, 37 bp, synthetic construct
  source 1..37: mol_type = other DNA
gatattgtcatgacacagaccccactgtccctgtccg

SEQ ID NO: 340 - DNA, 37 bp, synthetic construct
  source 1..37: mol_type = other DNA
gatatcgtcatgacacagaccccactgtccctgtccg

SEQ ID NO: 341 - DNA, 29 bp, synthetic construct
  source 1..29: mol_type = other DNA
gaggccgtgatgacgcagaccccactgtc

SEQ ID NO: 342 - DNA, 36 bp, synthetic construct
  source 1..36: mol_type = other DNA
gaaatcgtgatgacacagtctccagcctccctctcc

SEQ ID NO: 343 - DNA, 28 bp, synthetic construct
  source 1..28: mol_type = other DNA
ttgcccgtgctgacccagcctccaagtg

SEQ ID NO: 344 - DNA, 32 bp, synthetic construct
  source 1..32: mol_type = other DNA
caggctgtgctgactcagccaccctctgtgtc

SEQ ID NO: 345 - DNA, 35 bp, synthetic construct
  source 1..35: mol_type = other DNA
cagtctgccctgactcaaccttcctcggtgtctgg

SEQ ID NO: 346 - DNA, 35 bp, synthetic construct
  source 1..35: mol_type = other DNA
cagtctatgctgactcagccagcctcagtgtctgg

SEQ ID NO: 347 - DNA, 32 bp, synthetic construct
  source 1..32: mol_type = other DNA
cagtctgtgctgactcagccggcctcagtgtc

SEQ ID NO: 348 - DNA, 35 bp, synthetic construct
  source 1..35: mol_type = other DNA
cagtctgtgctgactcaaccagcctcagtgtccgg

SEQ ID NO: 349 - DNA, 34 bp, synthetic construct
  source 1..34: mol_type = other DNA
cagtctgtgctgactcagccgacctcagtgtcgg

SEQ ID NO: 350 - DNA, 34 bp, synthetic construct
  source 1..34: mol_type = other DNA
tcctatgtgctgtctcagccgccatcagcgactg

SEQ ID NO: 351 - DNA, 38 bp, synthetic construct
  source 1..38: mol_type = other DNA
tcctatgtgctgacacagctgccatccatgagtgtgac

SEQ ID NO: 352 - DNA, 25 bp, synthetic construct
  source 1..25: mol_type = other DNA
tgaggagacggtgaccagggtgccc

SEQ ID NO: 353 - DNA, 28 bp, synthetic construct
  source 1..28: mol_type = other DNA
tgaggagacagtgaccagggtgccctgg

SEQ ID NO: 354 - DNA, 28 bp, synthetic construct
  source 1..28: mol_type = other DNA
tgaggagacggtgaccagggttccctgg

SEQ ID NO: 355 - DNA, 29 bp, synthetic construct
  source 1..29: mol_type = other DNA
tgaggacacagtgaccagggtcccttggc

SEQ ID NO: 356 - DNA, 30 bp, synthetic construct
  source 1..30: mol_type = other DNA
tgaggacacgaagagtgaggtgccatggcc

SEQ ID NO: 357 - DNA, 28 bp, synthetic construct
  source 1..28: mol_type = other DNA
cggagcaggaaccaaggtggagctcaaa

SEQ ID NO: 358 - DNA, 27 bp, synthetic construct
  source 1..27: mol_type = other DNA
tttgatctccagtttggtcccttggcc

SEQ ID NO: 359 - DNA, 28 bp, synthetic construct
  source 1..28: mol_type = other DNA
tttgatctccaccttggtcccttggccg

SEQ ID NO: 360 - DNA, 27 bp, synthetic construct
  source 1..27: mol_type = other DNA
ggcaaagggacacatctggagattaaa

SEQ ID NO: 361 - DNA, 28 bp, synthetic construct
  source 1..28: mol_type = other DNA
gaggacggtcagctgggtcccttcaccg

SEQ ID NO: 362 - DNA, 28 bp, synthetic construct
  source 1..28: mol_type = other DNA
gaggatggtcagctgggtccctctgccg

SEQ ID NO: 363 - DNA, 28 bp, synthetic construct
  source 1..28: mol_type = other DNA
aaggacggtcagttgggttcctgagccg

SEQ ID NO: 364 - DNA, 26 bp, synthetic construct
  source 1..26: mol_type = other DNA
gaggacggtcagatgggtgcctccgc

SEQ ID NO: 365 - DNA, 25 bp, synthetic construct
  source 1..25: mol_type = other DNA
gaggacggtcaggtgggtgcctccg

SEQ ID NO: 366 - protein, 335 aa, Canis lupus
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 367 - protein, 110 aa, Canis lupus
RNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 368 - protein, 106 aa, Canis lupus
GQPKASPSVTLFPPSSEELGANKATLVCLISDFYPSGVTVAWKADGSPVTQGVETTKPSKQSNNKYAASSYLSLTPDKWKSHSSFSCLVTHEGSTVEKKVAPAECS

(19) United States
(12) Patent Application Publication
(10) Pub. No.: US 2026/0176615 A1
(43) Pub. Date: Jun. 25, 2026

(54) CANINE ANTIBODY LIBRARY

(71) Applicant: Invetx, Inc., Boston, MA (US)

(72) Inventors: William BRONDYK, Brewster, MA (US); Maxwell Alexander Stefan, San Mateo, CA (US); Hoa My Giang, Alameda, CA (US)

(21) Appl. No.: 19/426,613

(22) Filed: Dec. 19, 2025

Related U.S. Application Data
(60) Provisional application No. 63/736,105, filed on Dec. 19, 2024.

(51) Int. Cl. C12N 15/10; C07K 16/00; C40B 40/10

(52) U.S. Cl. CPC C12N 15/1037; C07K 16/00; C40B 40/10; C07K 2317/20; C07K 2317/565; C07K 2317/567

(57) ABSTRACT

The disclosure features polypeptide libraries (e.g., antibody libraries) for the selection of canine antibodies, and nucleic acids encoding said polypeptide libraries and polypeptides (e.g., antibodies or antigen-binding fragments thereof) derived from said polypeptide libraries. The disclosure also features methods for generating and using said polypeptide libraries.

### SEQUENCE LISTING

The instant application contains a Sequence Listing which has been submitted electronically in XML format and is hereby incorporated by reference in its entirety. Said XML copy, created on Dec. 18, 2025, is named “51682-014002_Sequence_Listing_1218_25” and is 326,658 bytes in size.

### FIELD OF INVENTION

This disclosure relates to polypeptide libraries (e.g., antibody libraries) for the selection of canine antibodies. The disclosure also relates to nucleic acids encoding said polypeptide libraries and polypeptides (e.g., antibodies or antigen-binding fragments thereof) derived from said polypeptide libraries. The disclosure further provides methods for generating and using said polypeptide libraries.

### BACKGROUND OF THE INVENTION

Therapeutic antibodies such as monoclonal antibodies have been used to treat and/or diagnose various human diseases, such as cancer, autoimmune diseases, metabolic diseases, and infectious diseases. While companion animals, such as dogs and cats, can develop diseases comparable to human diseases with a similar underlying pathology, the use of therapeutic antibodies to treat diseases in companion animals still represents an emerging field. Studies of companion animal diseases have revealed various target antigens that can be targeted by therapeutic antibodies for the treatment or diagnosis of diseases. However, the generation of species-specific antibodies for companion animals with desirable properties (e.g., optimal binding specificity, affinity, and half-life) remains technically challenging. There remains an unmet need for an efficient method for the discovery and optimization of therapeutic antibodies for use in companion animals.

Phage display has emerged as an effective molecular technique to generate vast polypeptide libraries, such as antibody libraries. Phage display involves batch cloning of DNA encoding for millions of variants of certain ligands (e.g., polypeptides or fragments thereof) into the phage genome as part of one of the phage coat proteins (e.g., pIII, pVI, or pVIII). Display of polypeptides on the surface of phage is achieved by fusion of the coding sequence of one of the coat proteins to the gene of interest, which enables the isolation of specific binding ligands by a series of recursive cycles of selection on a target antigen or ligand, with each cycle comprising the steps of binding, washing, elution, and amplification.

Based on the direct linkage between phenotype and genotype, phage display provides an expedient approach to study the genetics and functionality of the interacting polypeptides and to identify said polypeptides with desirable properties. While phage display has been used to generate antibody libraries for the development and optimization of human antibodies, the use of phage display for the development of therapeutic antibodies for companion animals is still under development. There is an unmet need for large phage display polypeptide libraries, and methods of constructing such libraries, for the purpose of developing therapeutic antibodies for companion animals.

### SUMMARY OF THE INVENTION

Provided herein, inter alia, are polypeptide libraries (e.g., synthetic canine polypeptide libraries) for the selection of canine antibodies or antigen-binding fragments thereof. The inventors have found that particular canine VH and VL combinations could lead to canine antibodies with desirable properties, such as high thermostability, high yield, and high efficiency for phage display.

In a first aspect, the disclosure features a polypeptide library comprising one or more polypeptide members, in which each polypeptide member in the library comprises:
(a) a heavy chain variable domain (VH) comprising VH framework region (FW) 1-heavy chain complementarity determining region (HCDR) 1-VH FW2-HCDR2-VH FW3-HCDR3-VH FW4 segments, in which:
   (i) the VH FW1 segment comprises an amino acid sequence that is at least 80% identical (e.g., at least 85%, 90%, 95%, 97%, 99%, or 100% identical) to the sequence of SEQ ID NO: 1 or SEQ ID NO: 12;
   (ii) the VH FW2 segment comprises an amino acid sequence that is at least 80% identical (e.g., at least 85%, 90%, 95%, 97%, 99%, or 100% identical) to the sequence of SEQ ID NO: 2 or SEQ ID NO: 13;
   (iii) the VH FW3 segment comprises an amino acid sequence that is at least 80% identical (e.g., at least 85%, 90%, 95%, 97%, 99%, or 100% identical) to the sequence of SEQ ID NO: 3 or SEQ ID NO: 14; and
   (iv) the VH FW4 segment comprises an amino acid sequence that is at least 80% identical (e.g., at least 85%, 90%, 95%, 97%, 99%, or 100% identical) to the sequence of SEQ ID NO: 4; and
(b) a light chain variable domain (VL) comprising VL FW1-light chain complementarity determining region (LCDR) 1-VL FW2-LCDR2-VL FW3-LCDR3-VL FW4 segments, in which:
   (i) the VL FW1 segment comprises the amino acid sequence that is at least 80% identical (e.g., at least 85%, 90%, 95%, 97%, 99%, or 100% identical) to a sequence selected from the group consisting of SEQ ID NO: 5, SEQ ID NO: 9, and SEQ ID NO: 15;
   (ii) the VL FW2 segment comprises the amino acid sequence that is at least 80% identical (e.g., at least 85%, 90%, 95%, 97%, 99%, or 100% identical) to a sequence selected from the group consisting of SEQ ID NO: 6, SEQ ID NO: 10, and SEQ ID NO: 16;
   (iii) the VL FW3 segment comprises the amino acid sequence that is at least 80% identical (e.g., at least 85%, 90%, 95%, 97%, 99%, or 100% identical) to a sequence selected from the group consisting of SEQ ID NO: 7, SEQ ID NO: 11, and SEQ ID NO: 17; and
   (iv) the VL FW4 segment comprises the amino acid sequence that is at least 80% identical (e.g., at least 85%, 90%, 95%, 97%, 99%, or 100% identical) to the of SEQ ID NO: 8.

In some embodiments, the VH FW1 segment comprises the amino acid sequence of SEQ ID NO: 1. In some embodiments, the VH FW1 segment comprises the amino acid sequence of SEQ ID NO: 12.

In some embodiments, the VH FW2 segment comprises the amino acid sequence of SEQ ID NO: 2. In some embodiments, the VH FW2 segment comprises the amino acid sequence of SEQ ID NO: 13.

In some embodiments, the VH FW3 segment comprises the amino acid sequence of SEQ ID NO: 3. In some embodiments, the VH FW3 segment comprises the amino acid sequence of SEQ ID NO: 14.

In some embodiments, the VH FW4 segment comprises the amino acid sequence of SEQ ID NO: 4.

In some embodiments, the VL FW1 segment comprises the amino acid sequence of SEQ ID NO: 5. In some embodiments, the VL FW1 segment comprises the amino acid sequence of SEQ ID NO: 9. In some embodiments, the VL FW1 segment comprises the amino acid sequence of SEQ ID NO: 15.

In some embodiments, the VL FW2 segment comprises the amino acid sequence of SEQ ID NO: 6. In some embodiments, the VL FW2 segment comprises the amino acid sequence of SEQ ID NO: 10. In some embodiments, the VL FW2 segment comprises the amino acid sequence of SEQ ID NO: 16.

In some embodiments, the VL FW3 segment comprises the amino acid sequence of SEQ ID NO: 7. In some embodiments, the VL FW3 segment comprises the amino acid sequence of SEQ ID NO: 11. In some embodiments, the VL FW3 segment comprises the amino acid sequence of SEQ ID NO: 17.

In some embodiments, the VL FW4 segment comprises the amino acid sequence of SEQ ID NO: 8.

In some embodiments, the VH FW1 segment comprises an amino acid sequence that is at least 80% identical (e.g., at least 85%, 90%, 95%, 97%, 99%, or 100% identical) to SEQ ID NO: 1, the VH FW2 segment comprises an amino acid sequence that is at least 80% identical (e.g., at least 85%, 90%, 95%, 97%, 99%, or 100% identical) to SEQ ID NO: 2, the VH FW3 segment comprises an amino acid sequence that is at least 80% identical (e.g., at least 85%, 90%, 95%, 97%, 99%, or 100% identical) to SEQ ID NO: 3, the VH FW4 segment comprises an amino acid sequence that is at least 80% identical (e.g., at least 85%, 90%, 95%, 97%, 99%, or 100% identical) to SEQ ID NO: 4, the VL FW1 segment comprises an amino acid sequence that is at least 80% identical (e.g., at least 85%, 90%, 95%, 97%, 99%, or 100% identical) to SEQ ID NO: 5, the VL FW2 segment comprises an amino acid sequence that is at least 80% identical (e.g., at least 85%, 90%, 95%, 97%, 99%, or 100% identical) to SEQ ID NO: 6, the VL FW3 segment comprises an amino acid sequence that is at least 80% identical (e.g., at least 85%, 90%, 95%, 97%, 99%, or 100% identical) to SEQ ID NO: 7, and the VL FW4 segment comprises an amino acid sequence that is at least 80% identical (e.g., at least 85%, 90%, 95%, 97%, 99%, or 100% identical) to SEQ ID NO: 8.

In some embodiments, the VH FW1 segment comprises the amino acid sequence of SEQ ID NO: 1, the VH FW2 segment comprises the amino acid sequence of SEQ ID NO: 2, the VH FW3 segment comprises the amino acid sequence of SEQ ID NO: 3, the VH FW4 segment comprises the amino acid sequence of SEQ ID NO: 4, the VL FW1 segment comprises the amino acid sequence of SEQ ID NO: 5, the VL FW2 segment comprises the amino acid sequence of SEQ ID NO: 6, the VL FW3 segment comprises the amino acid sequence of SEQ ID NO: 7, and the VL FW4 segment comprises the amino acid sequence of SEQ ID NO: 8.

In some embodiments, the VH FW1 segment comprises an amino acid sequence that is at least 80% identical (e.g., at least 85%, 90%, 95%, 97%, 99%, or 100% identical) to SEQ ID NO: 1, the VH FW2 segment comprises an amino acid sequence that is at least 80% identical (e.g., at least 85%, 90%, 95%, 97%, 99%, or 100% identical) to SEQ ID NO: 2, the VH FW3 segment comprises an amino acid sequence that is at least 80% identical (e.g., at least 85%, 90%, 95%, 97%, 99%, or 100% identical) to SEQ ID NO: 3, the VH FW4 segment comprises an amino acid sequence that is at least 80% identical (e.g., at least 85%, 90%, 95%, 97%, 99%, or 100% identical) to SEQ ID NO: 4, the VL FW1 segment comprises an amino acid sequence that is at least 80% identical (e.g., at least 85%, 90%, 95%, 97%, 99%, or 100% identical) to SEQ ID NO: 9, the VL FW2 segment comprises an amino acid sequence that is at least 80% identical (e.g., at least 85%, 90%, 95%, 97%, 99%, or 100% identical) to SEQ ID NO: 10, the VL FW3 segment comprises an amino acid sequence that is at least 80% identical (e.g., at least 85%, 90%, 95%, 97%, 99%, or 100% identical) to SEQ ID NO: 11, and the VL FW4 segment comprises an amino acid sequence that is at least 80% identical (e.g., at least 85%, 90%, 95%, 97%, 99%, or 100% identical) to SEQ ID NO: 8.

In some embodiments, the VH FW1 segment comprises the amino acid sequence of SEQ ID NO: 1, the VH FW2 segment comprises the amino acid sequence of SEQ ID NO: 2, the VH FW3 segment comprises the amino acid sequence of SEQ ID NO: 3, the VH FW4 segment comprises the amino acid sequence of SEQ ID NO: 4, the VL FW1 segment comprises the amino acid sequence of SEQ ID NO: 9, the VL FW2 segment comprises the amino acid sequence of SEQ ID NO: 10, the VL FW3 segment comprises the amino acid sequence of SEQ ID NO: 11, and the VL FW4 segment comprises the amino acid sequence of SEQ ID NO: 8.

In some embodiments, the VH FW1 segment comprises an amino acid sequence that is at least 80% identical (e.g., at least 85%, 90%, 95%, 97%, 99%, or 100% identical) to SEQ ID NO: 12, the VH FW2 segment comprises an amino acid sequence that is at least 80% identical (e.g., at least 85%, 90%, 95%, 97%, 99%, or 100% identical) to SEQ ID NO: 13, the VH FW3 segment comprises an amino acid sequence that is at least 80% identical (e.g., at least 85%, 90%, 95%, 97%, 99%, or 100% identical) to SEQ ID NO: 14, the VH FW4 segment comprises an amino acid sequence that is at least 80% identical (e.g., at least 85%, 90%, 95%, 97%, 99%, or 100% identical) to SEQ ID NO: 4, the VL FW1 segment comprises an amino acid sequence that is at least 80% identical (e.g., at least 85%, 90%, 95%, 97%, 99%, or 100% identical) to SEQ ID NO: 15, the VL FW2 segment comprises an amino acid sequence that is at least 80% identical (e.g., at least 85%, 90%, 95%, 97%, 99%, or 100% identical) to SEQ ID NO: 16, the VL FW3 segment comprises an amino acid sequence that is at least 80% identical (e.g., at least 85%, 90%, 95%, 97%, 99%, or 100% identical) to SEQ ID NO: 17, and the VL FW4 segment comprises an amino acid sequence that is at least 80% identical (e.g., at least 85%, 90%, 95%, 97%, 99%, or 100% identical) to SEQ ID NO: 8.

In some embodiments, the VH FW1 segment comprises the amino acid sequence of SEQ ID NO: 12, the VH FW2 segment comprises the amino acid sequence of SEQ ID NO: 13, the VH FW3 segment comprises the amino acid sequence of SEQ ID NO: 14, the VH FW4 segment comprises the amino acid sequence of SEQ ID NO: 4, the VL FW1 segment comprises the amino acid sequence of SEQ ID NO: 15, the VL FW2 segment comprises the amino acid sequence of SEQ ID NO: 16, the VL FW3 segment comprises the amino acid sequence of SEQ ID NO: 17, and the VL FW4 segment comprises the amino acid sequence of SEQ ID NO: 8.

In some embodiments, the library comprises at least 103 (e.g., at least 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, 10²¹, 10²², 10²³, 10²⁴, or 10²⁵) unique polypeptide members. In some embodiments, the library comprises from 10³ to 10⁴ unique polypeptide members. In some embodiments, the library comprises from 10³ to 10⁵ unique polypeptide members. In some embodiments, the library comprises from 10³ to 10⁶ unique polypeptide members. In some embodiments, the library comprises from 10³ to 10⁷ unique polypeptide members. In some embodiments, the library comprises from 10³ to 10⁸ unique polypeptide members. In some embodiments, the library comprises from 10³ to 10⁹ unique polypeptide members. In some embodiments, the library comprises from 10³ to 10¹⁰ unique polypeptide members. In some embodiments, the library comprises from 10³ to 10¹¹ unique polypeptide members. In some embodiments, the library comprises from 10³ to 10¹² unique polypeptide members. In some embodiments, the library comprises from 10³ to 10¹³ unique polypeptide members. In some embodiments, the library comprises from 10³ to 10¹⁴ unique polypeptide members. In some embodiments, the library comprises from 10³ to 10¹⁵ unique polypeptide members. In some embodiments, the library comprises from 10³ to 10¹⁶ unique polypeptide members. In some embodiments, the library comprises from 10³ to 10¹⁷ unique polypeptide members. In some embodiments, the library comprises from 10³ to 10¹⁸ unique polypeptide members. In some embodiments, the library comprises from 10³ to 10¹⁹ unique polypeptide members. In some embodiments, the library comprises from 10³ to 1.14E19 unique polypeptide members. In some embodiments, the library comprises from 10³ to 5.36E19 unique polypeptide members. In some embodiments, the library comprises from 10³ to 7.60E19 unique polypeptide members. In some embodiments, the library comprises from 10³ to 10²⁰ unique polypeptide members. In some embodiments, the library comprises from 10³ to 10²¹ unique polypeptide members. In some embodiments, the library comprises from 10³ to 10²² unique polypeptide members. In some embodiments, the library comprises from 10³ to 10²³ unique polypeptide members. In some embodiments, the library comprises from 10³ to 10²⁴ unique polypeptide members. In some embodiments, the library comprises from 10³ to 10²⁵ unique polypeptide members. In some embodiments, the library comprises from 10³ to 10²⁵ unique polypeptide members. In some embodiments, the library comprises at least 10⁴ unique polypeptide members. In some embodiments, the library comprises at least 10⁵ unique polypeptide members. In some embodiments, the library comprises at least 10⁶ unique polypeptide members. In some embodiments, the library comprises at least 10⁷ unique polypeptide members. In some embodiments, the library comprises at least 10⁸ unique polypeptide members. In some embodiments, the library comprises at least 10⁹ unique polypeptide members. In some embodiments, the library comprises at least 10¹⁰ unique polypeptide members.

In some embodiments, the HCDR1 segment comprises from 1 to 25 (e.g., 1, 2, 3, 4, 5, 6, 7, 8, 9, 10, 11, 12, 13, 14, 15, 16, 17, 18, 19, 20, 21, 22, 23, 24, or 25) amino acid residues, the HCDR2 segment comprises from 1 to 25 (e.g., 1, 2, 3, 4, 5, 6, 7, 8, 9, 10, 11, 12, 13, 14, 15, 16, 17, 18, 19, 20, 21, 22, 23, 24, or 25) amino acid residues, the HCDR3 segment comprises from 1 to 35 (e.g., 1, 2, 3, 4, 5, 6, 7, 8, 9, 10, 11, 12, 13, 14, 15, 16, 17, 18, 19,20,21,22,23,24,25,26,27,28,29,30,31,32,33,34, or 35) amino acid residues, the LCDR1 segment comprises from 1 to 25 (e.g., 1, 2, 3, 4, 5, 6, 7, 8, 9, 10, 11, 12, 13, 14, 15, 16, 17, 18, 19, 20, 21, 22, 23, 24, or 25) amino acid residues, the LCDR2 segment comprises from 1 to 25 (e.g., 1, 2, 3, 4, 5, 6, 7, 8, 9, 10, 11, 12, 13, 14, 15, 16, 17, 18, 19, 20, 21, 22, 23, 24, or 25) amino acid residues, and/or the LCDR3 segment comprises from 1 to 35 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35) amino acid residues.

In some embodiments, the HCDR1 segment comprises 10 amino acid residues, the HCDR2 segment comprises 14 amino acid residues, the HCDR3 segment comprises from 4 to 16 (e.g., 4, 5, 6, 7, 8, 9, 10, 11, 12, 13, 14, 15, or 16) amino acid residues, the LCDR1 segment comprises from 4 to 12 (e.g., 4, 5, 6, 7, 8, 9, 10, 11, or 12) amino acid residues, the LCDR2 segment comprises 8 amino acid residues, and/or the LCDR3 segment comprises from 9 to 12 (e.g., 9, 10, 11, or 12) amino acid residues.

In some embodiments, the HCDR1 segment comprises 10 amino acid residues, the HCDR2 segment comprises 14 amino acid residues, the HCDR3 segment comprises from 4 to 16 (e.g., 4, 5, 6, 7, 8, 9, 10, 11, 12, 13, 14, 15, or 16) amino acid residues, the LCDR1 segment comprises from 4 to 18 (e.g., 4, 5, 6, 7, 8, 9, 10, 11, 12, 13, 14, 15, 16, 17, or 18) amino acid residues, the LCDR2 segment comprises from 8 to 12 (e.g., 8, 9, 10, 11, or 12) amino acid residues, and/or the LCDR3 segment comprises from 9 to 13 (e.g., 9, 10, 11, 12, or 13) amino acid residues. In some embodiments, the LCDR2 segment comprises 8 or 12 amino acid residues.

In some embodiments, the HCDR1 segment comprises from 8 to 14 (e.g., 8, 9, 10, 11, 12, 13, or 14) amino acid residues, the HCDR2 segment comprises from 9 to 21 (e.g., 9, 10, 11, 12, 13, 14, 15, 16, 17, 18, 19, 20, or 21) amino acid residues, the HCDR3 segment comprises from 2 to 29 (e.g., 2, 3, 4, 5, 6, 7, 8, 9, 10, 11, 12, 13, 14, 15, 16, 17, 18, 19, 20, 21, 22, 23, 24, 25, 26, 27, 28, or 29) amino acid residues, the LCDR1 segment comprises from 5 to 16 (e.g., 5, 6, 7, 8, 9, 10, 11, 12, 13, 14, 15, or 16) amino acid residues, the LCDR2 segment comprises 7 or 8 amino acid residues, and/or the LCDR3 segment comprises from 5 to 16 (e.g., 5, 6, 7, 8, 9, 10, 11, 12, 13, 14, 15, or 16) amino acid residues.

In some embodiments, the HCDR1 segment comprises an amino acid sequence that is at least 80% identical (e.g., at least 85%, 90%, 95%, 97%, 99%, or 100% identical) to a sequence selected from the group consisting of SEQ ID NOS: 18-57, the HCDR2 segment comprises an amino acid sequence that is at least 80% identical (e.g., at least 85%, 90%, 95%, 97%, 99%, or 100% identical) to a sequence selected from the group consisting of SEQ ID NOS: 58-97, the HCDR3 segment comprises an amino acid sequence that is at least 80% identical (e.g., at least 85%, 90%, 95%, 97%, 99%, or 100% identical) to a sequence selected from the group consisting of SEQ ID NOS: 98-137, the LCDR1 segment comprises an amino acid sequence that is at least 80% identical (e.g., at least 85%, 90%, 95%, 97%, 99%, or 100% identical) to a sequence selected from the group consisting of SEQ ID NOS: 138-197; the LCDR2 segment comprises an amino acid sequence that is at least 80% identical (e.g., at least 85%, 90%, 95%, 97%, 99%, or 100% identical) to a sequence selected from the group consisting of SEQ ID NOS: 198-257; and/or the LCDR3 segment comprises an amino acid sequence that is at least 80% identical (e.g., at least 85%, 90%, 95%, 97%, 99%, or 100% identical) to a sequence selected from the group consisting of SEQ ID NOS: 258-317.

In some embodiments, the HCDR1 segment comprises an amino acid sequence selected from the group consisting of SEQ ID NOS: 18-57, the HCDR2 segment comprises an amino acid sequence selected from the group consisting of SEQ ID NOS: 58-97, the HCDR3 segment comprises an amino acid sequence selected from the group consisting of SEQ ID NOS: 98-137, the LCDR1 segment comprises an amino acid sequence selected from the group consisting of SEQ ID NOS: 138-197, the LCDR2 segment comprises an amino acid sequence selected from the group consisting of SEQ ID NOS: 198-257, and/or the LCDR3 segment comprises an amino acid sequence selected from the group consisting of SEQ ID NOS: 258-317.

In some embodiments, the HCDR1 segment comprises an amino acid sequence that is at least 80% identical (e.g., at least 85%, 90%, 95%, 97%, 99%, or 100% identical) to a sequence selected from the group consisting of SEQ ID NOS: 18-37, the HCDR2 segment comprises an amino acid sequence that is at least 80% identical (e.g., at least 85%, 90%, 95%, 97%, 99%, or 100% identical) to a sequence selected from the group consisting of SEQ ID NOS: 58-77, the HCDR3 segment comprises an amino acid sequence that is at least 80% identical (e.g., at least 85%, 90%, 95%, 97%, 99%, or 100% identical) to a sequence selected from the group consisting of SEQ ID NOS: 98-117, the LCDR1 segment comprises an amino acid sequence that is at least 80% identical (e.g., at least 85%, 90%, 95%, 97%, 99%, or 100% identical) to a sequence selected from the group consisting of SEQ ID NOS: 138-157; the LCDR2 segment comprises an amino acid sequence that is at least 80% identical (e.g., at least 85%, 90%, 95%, 97%, 99%, or 100% identical) to a sequence selected from the group consisting of SEQ ID NOS: 198-217; and/or the LCDR3 segment comprises an amino acid sequence that is at least 80% identical (e.g., at least 85%, 90%, 95%, 97%, 99%, or 100% identical) to a sequence selected from the group consisting of SEQ ID NOS: 258-277.

In some embodiments, the HCDR1 segment comprises an amino acid sequence selected from the group consisting of SEQ ID NOS: 18-37, the HCDR2 segment comprises an amino acid sequence selected from the group consisting of SEQ ID NOS: 58-77, the HCDR3 segment comprises an amino acid sequence selected from the group consisting of SEQ ID NOS: 98-117, the LCDR1 segment comprises an amino acid sequence selected from the group consisting of SEQ ID NOS: 138-157, the LCDR2 segment comprises an amino acid sequence selected from the group consisting of SEQ ID NOS: 198-217, and/or the LCDR3 segment comprises an amino acid sequence selected from the group consisting of SEQ ID NOS: 258-277.

In some embodiments, the HCDR1 segment comprises an amino acid sequence that is at least 80% identical (e.g., at least 85%, 90%, 95%, 97%, 99%, or 100% identical) to a sequence selected from the group consisting of SEQ ID NOS: 18-37, the HCDR2 segment comprises an amino acid sequence that is at least 80% identical (e.g., at least 85%, 90%, 95%, 97%, 99%, or 100% identical) to a sequence selected from the group consisting of SEQ ID NOS: 58-77, the HCDR3 segment comprises an amino acid sequence that is at least 80% identical (e.g., at least 85%, 90%, 95%, 97%, 99%, or 100% identical) to a sequence selected from the group consisting of SEQ ID NOS: 98-117, the LCDR1 segment comprises an amino acid sequence that is at least 80% identical (e.g., at least 85%, 90%, 95%, 97%, 99%, or 100% identical) to a sequence selected from the group consisting of SEQ ID NOS: 158-177; the LCDR2 segment comprises an amino acid sequence that is at least 80% identical (e.g., at least 85%, 90%, 95%, 97%, 99%, or 100% identical) to a sequence selected from the group consisting of SEQ ID NOS: 218-237; and/or the LCDR3 segment comprises an amino acid sequence that is at least 80% identical (e.g., at least 85%, 90%, 95%, 97%, 99%, or 100% identical) to a sequence selected from the group consisting of SEQ ID NOS: 278-297.

In some embodiments, the HCDR1 segment comprises an amino acid sequence selected from the group consisting of SEQ ID NOS: 18-37, the HCDR2 segment comprises an amino acid sequence selected from the group consisting of SEQ ID NOS: 58-77, the HCDR3 segment comprises an amino acid sequence selected from the group consisting of SEQ ID NOS: 98-117, the LCDR1 segment comprises an amino acid sequence selected from the group consisting of SEQ ID NOS: 158-177, the LCDR2 segment comprises an amino acid sequence selected from the group consisting of SEQ ID NOS: 218-237, and/or the LCDR3 segment comprises an amino acid sequence selected from the group consisting of SEQ ID NOS: 278-297.

In some embodiments, the HCDR1 segment comprises an amino acid sequence that is at least 80% identical (e.g., at least 85%, 90%, 95%, 97%, 99%, or 100% identical) to a sequence selected from the group consisting of SEQ ID NOS: 38-57, the HCDR2 segment comprises an amino acid sequence that is at least 80% identical (e.g., at least 85%, 90%, 95%, 97%, 99%, or 100% identical) to a sequence selected from the group consisting of SEQ ID NOS: 78-97, the HCDR3 segment comprises an amino acid sequence that is at least 80% identical (e.g., at least 85%, 90%, 95%, 97%, 99%, or 100% identical) to a sequence selected from the group consisting of SEQ ID NOS: 118-137, the LCDR1 segment comprises an amino acid sequence that is at least 80% identical (e.g., at least 85%, 90%, 95%, 97%, 99%, or 100% identical) to a sequence selected from the group consisting of SEQ ID NOS: 178-197; the LCDR2 segment comprises an amino acid sequence that is at least 80% identical (e.g., at least 85%, 90%, 95%, 97%, 99%, or 100% identical) to a sequence selected from the group consisting of SEQ ID NOS: 238-257; and/or the LCDR3 segment comprises an amino acid sequence that is at least 80% identical (e.g., at least 85%, 90%, 95%, 97%, 99%, or 100% identical) to a sequence selected from the group consisting of SEQ ID NOS: 298-317.

In some embodiments, the HCDR1 segment comprises an amino acid sequence selected from the group consisting of SEQ ID NOS: 38-57, the HCDR2 segment comprises an amino acid sequence selected from the group consisting of SEQ ID NOS: 78-97, the HCDR3 segment comprises an amino acid sequence selected from the group consisting of SEQ ID NOS: 118-137, the LCDR1 segment comprises an amino acid sequence selected from the group consisting of SEQ ID NOS: 178-197, the LCDR2 segment comprises an amino acid sequence selected from the group consisting of SEQ ID NOS: 238-257, and/or the LCDR3 segment comprises an amino acid sequence selected from the group consisting of SEQ ID NOS: 298-317.

In some embodiments, the VH is connected to the VL by a linker. In some embodiments, the linker comprises the amino acid sequence of SEQ ID NO: 331.

In some embodiments of any of the preceding aspects, the polypeptide library comprises one or more antibodies or antigen-binding fragments thereof.

In some embodiments, the antibody or antigen-binding fragment thereof is selected from the group consisting of a monoclonal antibody or antigen-binding fragment thereof, a polyclonal antibody or antigen-binding fragment thereof, a canine antibody or antigen-binding fragment thereof, a caninized antibody or antigen-binding fragment thereof, a bispecific antibody or antigen-binding fragment thereof, a multi-specific antibody or antigen-binding fragment thereof, a dual-variable immunoglobulin domain, a monovalent antibody or antigen-binding fragment thereof, a chimeric antibody or antigen-binding fragment thereof, a single-chain Fv molecule (scFv), a diabody, a triabody, a Fv fragment, a Fab fragment, a F(ab′)2 molecule, and a tandem scFv (taFv).

In another aspect, the disclosure features a collection of nucleic acids encoding any of the polypeptide libraries disclosed herein.

In another aspect, the disclosure features a collection of vectors comprising any of the collections of nucleic acids disclosed herein.

In another aspect, the disclosure features a collection of host cells comprising any of the collections of nucleic acids disclosed herein or any of the collections of vectors disclosed herein.

In another aspect, the disclosure features a method of isolating an antibody or antigen-binding fragment thereof that specifically binds to an antigen, the method comprising the steps of:
(a) contacting any of the polypeptide libraries disclosed herein with the antigen;
(b) removing the members of the polypeptide library that do not bind to the antigen; and
(c) recovering the members of the polypeptide library that bind to the antigen.

In another aspect, the disclosure features an antibody or antigen-binding fragment thereof isolated from any of the polypeptide libraries disclosed herein, or by any of the methods of isolating an antibody or antigen-binding fragment thereof disclosed herein.

### BRIEF DESCRIPTION OF THE DRAWINGS

FIGS. 1A-1C are a series of bar graphs showing the length distribution of CDR1 (FIG. 1A), CDR2 (FIG. 1B), and CDR3 (FIG. 1C) of canine germline VH (upper panel), VK (middle panel), and VL (lower panel) genes in peripheral blood mononuclear cells (PBMCs) from dogs determined by next-generation sequencing (NGS).

FIG. 2 is a bar graph showing the frequency distribution of different framework regions corresponding to canine germline VH, VK, or VL genes in PBMCs from dogs determined by NGS.

FIG. 3 is a series of bar graphs showing binding of recombinant SARS-CoV-2 S1 protein (S1 Delta) by polyclonal phages of the canine phage display libraries following four rounds (R) of selection.

Binding was measured by enzyme-linked immunosorbent assay (ELISA) and represented by raw OD450 values. Bovine serum albumin (BSA) was used as a control to determine background OD450.

FIG. 4 is a series of bar graphs showing the distribution of signal/background (S/B) ratio of an ELISA assay measuring the binding of recombinant SARS-CoV-1 S1 protein by individual phage clones obtained by screening the canine phage display libraries.

### DETAILED DESCRIPTION OF THE INVENTION

The invention described herein relates to polypeptide libraries (e.g., antibody libraries) for the selection of canine antibodies, nucleic acids encoding said polypeptide libraries, and polypeptides (e.g., antibodies or antigen-binding fragments thereof) derived from said polypeptide libraries. The invention also relates to methods for generating and using said polypeptide libraries (e.g., for isolating a polypeptide that specifically binds an antigen).

### Definitions

Where values are described in terms of ranges, it should be understood that the description includes the disclosure of all possible sub-ranges within such ranges, as well as specific numerical values that fall within such ranges irrespective of whether a specific numerical value or specific sub-range is expressly stated. All numerical designations, e.g., pH, KD, temperature, time, concentration, and molecular weight, including ranges, are approximations which are varied plus or minus (+/−) by increments of 1.0 or 0.1, as appropriate, or alternatively by a variation of +/− 15%, or alternatively 10%, or alternatively 5%, or alternatively 2%. It is to be understood, although not always explicitly stated, that all numerical designations are preceded by the term “about” and that a numerical designation may include numerical values that are rounded to the nearest significant figure. It also is to be understood, although not always explicitly stated, that the reagents described herein are merely exemplary and that equivalents of such are known in the art.

Unless otherwise defined, scientific and technical terms used in connection with the present disclosure shall have meanings that are commonly understood by those of ordinary skill in the art.

Further, unless otherwise required by context or expressly indicated, singular terms shall include pluralities and plural terms shall include the singular. For any conflict in definitions between various sources or publications, the definition provided herein will control.

It is understood that embodiments of the invention described herein include “comprising,” “consisting,” and “consisting essentially of” aspects and embodiments. As used herein, the singular form “a”, “an,” and “the” includes plural references (e.g., at least one, one or more) unless indicated otherwise. The use of the term “or” herein means “and/or” and is not meant to imply that alternatives are mutually exclusive unless specified otherwise.

As used herein when referring to a measurable value such as an amount or concentration and the like, the term “about” is meant to encompass variations of 20%, 10%, 5%, 1%, 0.5%, or even 0.1% of the specified amount. The term “about” can also be used to describe range.

As used herein, the terms “percent (%) sequence identity,” “% identical,” and “homology” with respect to a nucleic acid or polypeptide sequence are defined as the percentage of nucleotides or amino acid residues in a reference sequence that are identical with the nucleotides or amino acid residues in the specific nucleic acid or polypeptide sequence, after aligning the sequences and introducing gaps, if necessary, to achieve the maximum percent sequence identity, and not considering any conservative substitutions as part of the sequence identity. Alignment for purposes of determining percent sequence identity can be achieved in various ways that are within the skill in the art, for instance, using publicly available computer software such as BLAST, BLAST-2, CLUSTAL OMEGA, ALIGN, or MEGALIGN™ (DNASTAR) software. Those skilled in the art can determine appropriate parameters for measuring alignment, including any parameters needed to achieve maximal alignment over the full length of sequences being compared. In some embodiments, a variant has at least 50% sequence identity with the reference nucleic acid molecule or polypeptide after aligning the sequences and introducing gaps, if necessary, to achieve the maximum percent sequence identity, and not considering any conservative substitutions as part of the sequence identity. Such variants include, for instance, polypeptides wherein one or more amino acid residues are added or deleted at the N- or C-terminus of the polypeptide. In some embodiments, a variant has at least 50% sequence identity, at least 60% sequence identity, at least 65% sequence identity, at least 70% sequence identity, at least 75% sequence identity, at least 80% sequence identity, at least 85% sequence identity, at least 90% sequence identity, at least 95% sequence identity, at least 97% sequence identity, at least 98% sequence identity, or at least 99% sequence identity with the sequence of the reference nucleic acid or polypeptide.

As used herein, the term “amino acid substitution” refers to the replacement of one amino acid in a polypeptide with another amino acid. In some embodiments, an amino acid substitution is a conservative substitution. Amino acid substitutions may be introduced into a polypeptide screened for a desired activity, for example, retained or improved binding to FcRn, retained or improved antigen binding, decreased immunogenicity, improved ADCC or CDC, or enhanced pharmacokinetics.

As used herein, the term “conservative substitution” as used herein refers to a substitution of one amino acid residue for another amino acid residue that has similar properties such as charge, hydrophobicity, and/or size. For example, amino acids may be grouped according to common side-chain properties:
Hydrophobic: e.g., Norleucine (Nle), Met, Ala, Val, Leu, and lie;
Neutral hydrophilic: e.g., Cys, Ser, Thr, Asn, and Gln;
Acidic: e.g., Asp, and Glu;
Basic: e.g., His, Lys, and Arg;
Rigid: e.g., Gly, and Pro; and
Aromatic: e.g., Trp, Tyr, and Phe.

Conservative substitutions will entail exchanging a member of one of these classes with another member of the same class. Non-conservative substitutions will entail exchanging a member of one of these classes with another class. In some embodiments, a conservative amino acid substitution refers to a substitution that results in similar properties or functions as another amino acid substitution. For example, a conservative amino acid substitution of A426Y can be A426F, A426T, or A426W. Additional, nonlimiting examples for conservative amino acid substitutions are shown in Table 1.

TABLE 1
Examples for conservative amino acid substitutions

| Original residue | Exemplary conservative substitutions |
|---|---|
| Ala (A) | Gly; Val; Leu; Ile; Ser |
| Arg (R) | Lys; His; Gln; Asn |
| Asn (N) | Gln; His; Asp; Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Nle |
| Leu (L) | Nle; Ile; Val; Met; Ala; Phe |
| Lys (K) | Arg; His; Gln; Asn |
| Met (M) | Leu; Phe; Ile; Tyr |
| Phe (F) | Trp; Leu; Val; Ile; Ala; Tyr; Met |
| Pro (P) | Ala; Gly |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Nle |

As used herein, the term “affinity” refers to the strength of the sum total of noncovalent interactions between a single binding site of a molecule (e.g., an antibody or a receptor) and its binding partner (e.g., an antigen or a ligand). Unless indicated otherwise, as used herein, “binding affinity” refers to intrinsic binding affinity which reflects a 1:1 interaction between members of a binding pair (e.g., antibody and antigen, receptor and ligand). The affinity of a molecule X for its partner Y can generally be represented by the dissociation constant (KD). Affinity can be measured by common protein-protein interaction tools known in the art, such as, for example, immunoblot, enzyme-linked immunosorbent assay (ELISA), kinetic exclusion assay (KINExA®), biolayer interferometry (BLI), or surface plasmon resonance (SPR) devices. Specific illustrative and exemplary embodiments for measuring binding affinity are described in the following.

The term “surface plasmon resonance (SPR)” denotes an optical phenomenon that allows for the analysis of real-time biospecific interactions by detection of alterations in protein concentrations within a biosensor matrix, for example, using the BIAcore™ system (BIAcore International AB, a GE Healthcare company, Uppsala, Sweden and Piscataway, N.J.). For further descriptions, see Jonsson et al., 1993, Ann. Biol. Clin. 51: 19-26.

As user herein, the term “amino acid sequence” refers to a sequence of amino acids residues in a peptide or protein. The terms “polypeptide” and “protein” are used interchangeably to refer to a polymer of amino acid residues and are not limited to a minimum length. Such polymers of amino acid residues may contain natural or unnatural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to polymerase chain reaction (PCR) amplification.

As used herein, the term “antibody” herein is used in the broadest sense and refers to various antibody structures, including but not limited to monoclonal antibodies, polyclonal antibodies, multispecific antibodies (e.g., bispecific antibodies), and antibody fragments (e.g., Fab) so long as they exhibit the desired antigen-binding activity. An antibody may contain at least one heavy chain and/or at least one light chain. The heavy and light chains of an antibody may be interconnected by disulfide bonds. Each heavy chain of an antibody may contain a heavy chain variable region (VH) and a heavy chain constant region. The heavy chain constant region of a naturally occurring antibody (e.g., an IgG antibody) may comprise CH1, CH2, and CH3 domains. Each light chain of an antibody may contain a light chain variable region (VL) and a light chain constant region. The light chain constant region of a naturally occurring antibody (e.g., an IgG antibody) may comprise a CL domain. The variable regions of the heavy and light chains of an antibody may contain a binding domain that interacts with an antigen. The constant regions of an antibody may mediate the binding of the antibody to host tissues or factors, such as various cells of the immune system (e.g., effector cells) and the components of the classical complement system (e.g., C1q). The VH and VL regions can be further divided into regions of hypervariability, or complementarity determining regions (CDRs), interspersed with regions that are relatively conserved, or framework regions (FWs). The FWs of an antibody serve to position and align the CDRs, which are primarily responsible for binding specificity to an antigen. Each VH and VL comprises three CDRs and four FWs arranged (from amino-terminus to carboxy-terminus) in the following order: FW1-CDR1-FW2-CDR2-FW3-CDR3-FW4. The term “bispecific antibody” refers to an antibody derivative that has, in the same antibody molecule, variable regions that recognize two different epitopes. A bispecific antibody may be an antibody that recognizes two different antigens, or an antibody that recognizes two different epitopes on the same antigen.

As used herein, the term “canine antibody” refers to antibodies having variable regions in which one or more of the FWs and the CDRs are derived from sequences of canine origin. For example, both of the FWs and the CDRs may be derived from sequences of canine origin. Furthermore, if the antibody contains one or more constant regions, the constant regions may also be derived from canine sequences, such as canine germline sequences, or mutated versions thereof. The canine antibodies of the disclosure may also include one or more amino acid residues not encoded by canine sequences (e.g., due to mutations introduced by mutagenesis in vitro or somatic mutations in vivo).

As used herein, the terms “antibody fragment” or “antigen-binding fragment” refer to a molecule other than a full-length antibody that comprises a portion of a full-length antibody that binds the antigen to which the full-length antibody binds. Antigen binding functions of an antibody can be performed by fragments of an intact antibody. In some embodiments, antibody fragments include but are not limited to Fab; single chain variable fragment (e.g., scFv); Fv; Fab′; Fab′-SH; F(ab′)₂; nanobody; diabody; and multispecific antibodies formed from antibody fragments.

As used herein, the terms “full-length antibody” and “whole antibody” are used herein interchangeably to refer to an antibody having a structure substantially similar to a native antibody structure or having heavy chains that contain an Fc region as defined herein.

As used herein, the term “monoclonal antibody” refers to a composition of antibody molecules of single molecular composition. A monoclonal antibody composition displays a single binding specificity and affinity for a particular epitope.

As used herein, the terms “nanobody,” “VHH,” “VHH domain,” “VHH antibody fragment,” and “single domain antibody” as interchangeably used herein denote the variable domain of the single heavy chain of antibodies of the type of those found in Camelidae, which are typically found in natural form to lack light chains. Suitable nanobodies will be familiar to persons skilled in the art, illustrated examples of which include nanobodies of camels, dromedaries, llamas, and alpacas. However, the single domain antibody may be from non-Camelidae sources as well.

As used herein, the term “isotype” refers to the antibody molecular class (e.g., IgA, IgD, IgE, IgG, and IgM) that is determined by the heavy chain constant region. Each isotype may be subdivided into different subclasses of distinct structural features. For example, in canine, there are four IgG subclasses: IgGA, IgGB, IgGC, and IgGD. An engineered antibody having an Fc region variant, or modifications at the Fc domain, e.g., to enhance or reduce effector functions or binding affinity to Fc receptors, is considered having the same isotype as the corresponding wild type antibody.

As used herein, the term “binding domain” refers to a part of a compound or a molecule that specifically binds to a target epitope, antigen, ligand, or receptor. Binding domains include but are not limited to antibodies (e.g., monoclonal, polyclonal, recombinant, and chimeric antibodies), antibody fragments or portions thereof (e.g., Fab, scFv, Fv, Fab′, Fab′-SH, F(ab′)₂, nanobody, and diabody), receptors or fragments thereof (e.g., an extracellular domain of a canine receptor protein), ligands, aptamers, and other molecules having an identified binding partner.

As used herein, the term “chimeric” antibody refers to an antibody in which a portion of the heavy and/or light chain is derived from a particular source or species, while the remainder of the heavy and/or light chain is derived from a different source or species.

As used herein, the terms “Fc region,” “Fc domain,” and “Fc polypeptide” refer to a C-terminal region of an immunoglobulin heavy chain that contains at least a portion of the constant region. The term “Fc region variant” refers to a variant of the Fc region of a canine having a substitution or substitutions relative to the wild type canine Fc region. The term “Fc domain of the wild type canine IgG” refers to the native Fc region of a canine antibody. The term “canine Fc region variant” refers to a variant of the Fc region of a canine antibody having a substitution or substitutions relative to the wild type canine Fc region. In some embodiments, the canine Fc region sequences are from a canine (e.g., dog) IgG (e.g., IgGA, IgGB, IgGC, or IgGD). In some embodiments, the IgG Fc polypeptide comprises the hinge, CH2, and CH3, but does not comprise CH1 or CL. In some embodiments, the IgG Fc polypeptide comprises CH2 and CH3, but does not comprise CH1, the hinge, or CL. In some embodiments, the IgG Fc polypeptide comprises CH1, hinge, CH2, and CH3, with or without CL. In some embodiments, the IgG Fc polypeptide comprises CH1, hinge, CH2, CH3, and CL. For example, CL may be linked to CH1 via a disulfide bridge. In some embodiments, an Fc polypeptide, such as an IgG Fc polypeptide, lacks one or more C-terminal amino acids, such as 1 to 20, 1 to 15, 1 to 10, 1 to 5, or 1 to 2 amino acids, while retaining biological activity. In some embodiments, the biological activity of an Fc polypeptide is the ability to bind FcRn. Unless otherwise specified herein, numbering of amino acid residues in the Fc region or constant region is according to the EU numbering system, also called the EU index, as described in Kabat et al. Sequences of Proteins of Immunological Interest, 5th Ed. Public Health Service, National Institutes of Health, Bethesda, MD, 1991.

As used herein, the term “Fc domain monomer” refers to a polypeptide chain that includes at least a hinge domain and second and third antibody constant domains (CH2 and CH3) or functional fragments thereof (e.g., fragments that are capable of (i) dimerizing with another Fc domain monomer to form an Fc domain and (ii) binding to an Fc receptor). The Fc domain monomer can be any immunoglobulin antibody isotype, including, e.g., IgG. Additionally, the Fc domain monomer can be an IgG isotype, e.g., IgGA, IgGB, IgGC, or IgGD in dogs. In some embodiments, an Fc domain monomer does not include any portion of an immunoglobulin that is capable of antigen binding, e.g., a variable domain or a CDR. In some embodiments, an Fc domain monomer includes a portion of an immunoglobulin that can act as a binding domain, e.g., a variable domain or a CDR. In some embodiments, an Fc domain monomer includes a single-domain antibody, e.g., a VHH domain.

As used herein, the term “Fc construct” refers to associated polypeptide chains that includes Fc domain monomers or Fc region variants as described herein (e.g., an Fc construct comprising Fc domain monomers or Fc region variants). Fc constructs described herein can include Fc domain monomers that have the same or different sequences. In some embodiments, an Fc construct does not include any portion of an immunoglobulin that is capable of antigen binding, e.g., a variable domain or a CDR. In some embodiments, an Fc construct includes a portion of an immunoglobulin that can act as an antigen-recognition region, e.g., a variable domain or a CDR. In some embodiments, an Fc construct includes a single-domain antibody, e.g., a VHH domain.

As used herein, the term “library” refers to a non-naturally occurring collection of distinct molecules. While individual molecules of a library may naturally occur, the entire collection of molecules constituting a library according to the invention does not naturally occur in a native, non-engineered organism. A library in the context of the present disclosure is a mixture of heterogeneous polypeptides or nucleic acids. The library is composed of members, each of which have a single polypeptide or nucleic acid sequence. Sequence differences between library members are responsible for the diversity present in the library. The library may take the form of a simple mixture of polypeptides or nucleic acids, or may be in the form of an organism or a cell (e.g., a bacterium, a virus, a bacteriophage, an animal cell, or a plant cell) transformed with a library of nucleic acids and/or expressing the library of polypeptides. In some embodiments, the nucleic acids are incorporated into vectors. For example, the nucleic acids may be incorporated into expression vectors to allow expression of the polypeptides encoded by the nucleic acids. In a certain aspect, a library may take the form of a population of host organisms or cells, in which each organism or cell contains one or more copies of an expression vector containing a single member of the library in nucleic acid form which can be expressed to produce its corresponding polypeptide member.

Thus, the population of host organisms or cells has the potential to encode a large collection of genetically diverse polypeptide variants. Antibody libraries can be derived from immunoglobulins or fragments thereof that are biased towards specific antigen. Such libraries may be prepared by isolating immunoglobulins or fragments thereof from immunized animals (e.g., immunized canines). Alternatively, antibody libraries can be derived from naive immunoglobulins or fragments thereof (i.e., immunoglobulins that are not biased towards specific antigen). Such libraries are referred to as “unbiased” libraries. In some embodiments, antibody libraries are constructed with VH and VL gene pools that are cloned from B cells isolated from an animal (e.g., a canine). When said animal is not immunized, it is possible to generate unbiased, naive antibody libraries. Alternatively, unbiased, naive antibody libraries can also be generated using a synthetic approach, where the library is constructed entirely in vitro, e.g. using recombinant DNA technology. Such antibody libraries are referred to as “synthetic” antibody libraries. As used herein, the term “fully synthetic” library refers to antibody libraries that are completely, de novo constructed by DNA synthesis or related DNA technologies. In such libraries, the entire collection of DNA, i.e., the nucleic acids encoding the CDRs, as well as the surrounding regions (e.g. the FWs), of the antibodies of the library, is constructed de novo. The terms “synthetic” and “fully synthetic” therefore refer to the de novo origin of the nucleic acids. In contrast, a “semi-synthetic” antibody library encodes or contains antibodies that are partially constructed de novo. For example, a semi-synthetic library may contain antibodies whose one or more CDRs are synthesized de novo, whereas the remaining components, e.g., the remaining CDRs and/or FWs, are derived from natural sources.

As used herein, the term “unique” refers to a sequence that is different (e.g., has a different chemical structure) from every other sequence within the defined collection of sequences (e.g., the theoretical diversity). It is understood that there are likely more than one copy of many unique sequences from the theoretical diversity in a particular collection of sequences. For example, a polypeptide library comprising three unique polypeptide sequences at the theoretical level may comprise nine total polypeptide members if each sequence occurs three times in the library. In some embodiments, each unique sequence may occur only once, less than once, or more than once.

As used herein, the term “VH/VL combination” refers to the combination (e.g., pairing) of one VH polypeptide member and one VL polypeptide member from a polypeptide library of the disclosure. The pairing may be between different domains of one polypeptide chain comprising the VH polypeptide and the VL polypeptide, as in, e.g., an scFv, or between two polypeptide chains comprising the VH polypeptide and the VL polypeptide, as in, e.g., a Fab fragment.

As used herein, the term “wild type” refers to a non-mutated version of a polypeptide that occurs in nature, or a fragment thereof. A wild type polypeptide may be produced recombinantly.

As used herein, the term “germline” refers to fully germline sequences and in addition to germline sequences that have been modified or engineered with minor mutations in the amino acid sequence. For example, minor mutations may be introduced intentionally to such germline sequences to facilitate manipulation or expression. Minor mutations may also be caused by errors in synthesis, amplification, or cloning.

As used herein, the term “effector functions” refers to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g., B cell receptor); and B cell activation.

As used herein, the terms “nucleic acid” and “polynucleotide,” which are used interchangeably, refer to deoxyribonucleotides, ribonucleotides, and polymers thereof, in either single- or double-stranded form. The terms encompass modified nucleic acids containing known base analogs or modified sugar-phosphate backbone residues or linkages, which are naturally occurring or non-naturally occurring while having similar binding properties as a wild type nucleic acid, and which may be metabolized in a manner similar to a wild type nucleic acid.

As used herein, the term “vector” refers to a nucleic acid molecule capable of propagating another nucleic acid to which it is linked. The term includes the vector as a self-replicating nucleic acid structure as well as the vector incorporated into the genome of a host cell into which it has been introduced. Certain vectors can direct the expression of nucleic acids to which they are operatively linked, and such vectors are referred to herein as “expression vectors.” An expression vector comprises a recombinant nucleic acid comprising expression control sequences operatively linked to a nucleic caid sequence to be expressed. An expression vector comprises sufficient elements (e.g., cis-acting elements) for expression of a nucleic acid. Exemplary expression vectors include, but are not limited to cosmids, plasmids, and viruses (e.g., lentiviruses, retroviruses, adenoviruses, and adeno-associated viruses) that incorporate the one or more recombinant nucleic acids.

As used herein, the terms “host cell” and “host cell culture” are used interchangeably and refer to cells into which exogenous nucleic acid has been introduced, including the progeny of such cells. Host cells include bacterial (e.g., E. coli cells) and eukaryotic cells. In some embodiments, host cells include yeast cells (e.g., Pichia (see, e.g., Powers et al., 2001, J Immunol Methods. 251: 123-135), Hanseula, or Saccharomyces). In some embodiments, host cells also include “transformants” and “transformed cells,” which include the primary transformed cell lines (e.g., CHO, 293E, COS, 293T, and HeLa) and progeny derived therefrom without regard to the number of passages. Progeny may not be completely identical in nucleic acid content to a parent cell but may contain mutations. Mutant progeny that has the same function or biological activity as screened or selected for in the originally transformed cell are included herein.

As used herein, the term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

As used herein, the term “epitope” refers to the particular site or sites on an antigen molecule to which an antibody or other binding agent binds. For example, an epitope may be a linear epitope or a conformational epitope.

As used herein, the terms “reduce” and “inhibit” refer to an overall decrease, for example, of 20% or greater, of 50% or greater, or of 75%, 85%, 90%, 95%, or greater, e.g., as compared to a reference or control.

As used herein, the terms “increase” and “enhance” refer to an overall increase, for example, of 20% or greater, of 50% or greater, or of 75%, 85%, 90%, 95%, or greater, e.g., as compared to a reference or control.

As used herein, a polypeptide “variant” refers to a polypeptide that differs from a reference polypeptide by single or multiple non-native amino acid substitutions, deletions, and/or additions. In some embodiments, a variant retains at least one biological activity of the reference polypeptide. In some embodiments, a variant has a biological activity that the reference polypeptide substantially lacks. A “canine Fc region variant” comprises an amino acid sequence which differs from that of a wild type canine Fc region by at least one amino acid modification, preferably one or more amino acid substitution(s). Preferably, the canine Fc region variant has at least one amino acid substitution compared to a wild type canine Fc region, e.g., from one to ten amino acid substitutions, and preferably from one to five amino acid substitutions in a wild type canine Fc region. The canine Fc region variant herein will preferably possess at least 80% homology with a wild type canine Fc region, and most preferably at least 90% homology therewith, more preferably at least 95% homology therewith. In some embodiments, the canine IgG Fc region is a canine IgGA Fc region variant, a canine IgGB Fc region variant, a canine IgGC Fc region variant, or a canine IgGD Fc region variant. In some embodiments, the Fc region variant (e.g., a canine Fc region variant) comprises the hinge, CH2, and CH3, but does not comprise CH1 or CL. In some embodiments, the Fc region variant (e.g., a canine Fc region variant) comprises CH2 and CH3, but does not comprise CH1, the hinge, or CL. In some embodiments, the Fc region variant (e.g., a canine Fc region variant) comprises CH1, hinge, CH2, and CH3, with or without CL. In some embodiments, the Fc region variant (e.g., a canine Fc region variant) comprises CH1, hinge, CH2, CH3, and CL. For example, CL may be linked to CH1 via a disulfide bridge.

### Library Construction

A polypeptide library of the disclosure may contain VH polypeptide members derived from a germline canine IgG VH region. In some embodiments, VH polypeptide members may be derived from a sequence that is at least 80% identical (e.g., at least 85%, 90%, 95%, 97%, 99%, or 100% identical) to the germline canine IgG VH region CanVH1 or CanVH2. The amino acid sequences of CanVH1 and CanVH2 are provided below, in which the amino acid residues corresponding to the highly diverse heavy chain CDR3 (HCDR3) are replaced with “X”:

CanVH1
(SEQ ID NO: 318):
EVQLVESGGDLVKPGGSLRLSCVASGFTFSSYHMSWVRQAPGKGL
QWVAYINSGGSSTSYADAVKGRFTISRDNAKNTLYLQMNSLRAED
TAVYYCXXXXXXWGQGTLVTVSS
CanVH2
(SEQ ID NO: 319):
ELTLQESGPGLVKPSQTLSLTCVVSGGSVTSSYYWNWIRQRPGRG
LEWMGYWTGSTNYNPAFQGRISITADTAKNQFSLQLSSMTTEDTA
VYYCXXXXXXWGQGTLVTVSS

A polypeptide library of the disclosure may contain VL polypeptide members derived from a germline canine IgG VL region. In some embodiments, VL polypeptide members may be derived from a sequence that is at least 80% identical (e.g., at least 85%, 90%, 95%, 97%, 99%, or 100% identical) to a germline canine IgG VL region selected from the group consisting of CanVL1, CanVL2, and CanVL3. The amino acid sequences of CanVL1, CanVL2, and CanVL3 are provided below, in which the amino acid residues corresponding to the highly diverse light chain CDR3 (LCDR3) are replaced with “X”:

CanVL1
(SEQ ID NO: 320):
QSVLTQPTSVSGSLGQRVTISCSGSTNNIGIVGASWYQQLPGKAP
KLLVDSDGDRPSGVPDRFSGSKSGNSATLTITGLQAEDEADYYCQ
XXXXXXFGGGTHLTVL
CanVL2
(SEQ ID NO: 321):
QAVLTQPPSVSAALGQRVTISCTGSNTNIGSGYDVQWYQQLPGKS
PKTIIYGNSNRPSGVPVRFSGSKSGSTATLTITGIQAEDEADYYC
QXXXXXXFGGGTHLTV
CanVL3
(SEQ ID NO: 322):
QSMLTQPASVSGSLGQKVTISCTGSSSNIGGNYVGWYQQLPGIGP
RTVIYGNNYRPSGVPDRFSGSKSGSSATLTISGLQAEDEAEYYCS
XXXXXXFGGGTHLTV

In some embodiments, the polypeptide library comprises VH polypeptide members derived from a sequence that is at least 80% identical (e.g., at least 85%, 90%, 95%, 97%, 99%, or 100% identical) to CanVH1 and VL polypeptide members derived from a sequence that is at least 80% identical (e.g., at least 85%, 90%, 95%, 97%, 99%, or 100% identical) to CanVL1. In some embodiments, the polypeptide library comprises VH polypeptide members derived from CanVH1 and VL polypeptide members derived from CanVL1.

In some embodiments, the polypeptide library comprises VH polypeptide members derived from a sequence that is at least 80% identical (e.g., at least 85%, 90%, 95%, 97%, 99%, or 100% identical) to CanVH1 and VL polypeptide members derived from a sequence that is at least 80% identical (e.g., at least 85%, 90%, 95%, 97%, 99%, or 100% identical) to CanVL2. In some embodiments, the polypeptide library comprises VH polypeptide members derived from CanVH1 and VL polypeptide members derived from CanVL2.

In some embodiments, the polypeptide library comprises VH polypeptide members derived from a sequence that is at least 80% identical (e.g., at least 85%, 90%, 95%, 97%, 99%, or 100% identical) to CanVH2 and VL polypeptide members derived from a sequence that is at least 80% identical (e.g., at least 85%, 90%, 95%, 97%, 99%, or 100% identical) to CanVL3. In some embodiments, the polypeptide library comprises VH polypeptide members derived from CanVH2 and VL polypeptide members derived from CanVL3.

In some embodiments, the polypeptide library of the disclosure comprises VH polypeptide members comprising VH FW1-HCDR1-VH FW2-HCDR2-VH FW3-HCDR3-VH FW4 segments.

In some embodiments, the VH FW1 segment comprises an amino acid sequence that is at least 80% identical (e.g., at least 85%, 90%, 95%, 97%, 99%, or 100% identical) to the sequence of:

(a)
(SEQ ID NO: 1)
EVQLVESGGDLVKPGGSLRLSCVAS;
or
(b)
(SEQ ID NO: 12)
ELTLQESGPGLVKPSQTLSLTCVVS.

In some embodiments, the VH FW1 segment comprises the amino acid sequence of SEQ ID NO: 1. In some embodiments, the VH FW1 segment comprises the amino acid sequence of SEQ ID NO: 12.

In some embodiments, the VH FW2 segment comprises an amino acid sequence that is at least 80% identical (e.g., at least 85%, 90%, 95%, 97%, 99%, or 100% identical) to the sequence of:

(a)
(SEQ ID NO: 2)
WVRQAPGKGLQW;
or
(b)
(SEQ ID NO: 13)
WIRQRPGRGLEW.

In some embodiments, the VH FW2 segment comprises the amino acid sequence of SEQ ID NO: 2. In some embodiments, the VH FW2 segment comprises the amino acid sequence of SEQ ID NO: 13.

In some embodiments, the VH FW3 segment comprises an amino acid sequence that is at least 80% identical (e.g., at least 85%, 90%, 95%, 97%, 99%, or 100% identical) to the sequence of:

(a)
(SEQ ID NO: 3)
DAVKGRFTISRDNAKNTLYLQMNSLRAEDTAVYYC;
or
(b)
(SEQ ID NO: 14)
PAFQGRISITADTAKNQFSLQLSSMTTEDTAVYYC.

In some embodiments, the VH FW3 segment comprises the amino acid sequence of SEQ ID NO: 3. In some embodiments, the VH FW3 segment comprises the amino acid sequence of SEQ ID NO: 14.

In some embodiments, the VH FW4 segment comprises an amino acid sequence that is at least 80% identical (e.g., at least 85%, 90%, 95%, 97%, 99%, or 100% identical) to the sequence of WGQGTLVTVSS (SEQ ID NO: 4).

In some embodiments, the VH FW4 segment comprises the amino acid sequence of SEQ ID NO: 4.

In some embodiments, the VH FW1 segment comprises an amino acid sequence that is at least 80% identical (e.g., at least 85%, 90%, 95%, 97%, 99%, or 100% identical) to SEQ ID NO: 1, the VH FW2 segment comprises an amino acid sequence that is at least 80% identical (e.g., at least 85%, 90%, 95%, 97%, 99%, or 100% identical) to SEQ ID NO: 2, the VH FW3 segment comprises an amino acid sequence that is at least 80% identical (e.g., at least 85%, 90%, 95%, 97%, 99%, or 100% identical) to SEQ ID NO: 3, and the VH FW4 segment comprises an amino acid sequence that is at least 80% identical (e.g., at least 85%, 90%, 95%, 97%, 99%, or 100% identical) to SEQ ID NO: 4.

In some embodiments, the VH FW1 segment comprises the amino acid sequence of SEQ ID NO: 1, the VH FW2 segment comprises the amino acid sequence of SEQ ID NO: 2, the VH FW3 segment comprises the amino acid sequence of SEQ ID NO: 3, and the VH FW4 segment comprises the amino acid sequence of SEQ ID NO: 4.

In some embodiments, the VH FW1 segment comprises an amino acid sequence that is at least 80% identical (e.g., at least 85%, 90%, 95%, 97%, 99%, or 100% identical) to SEQ ID NO: 12, the VH FW2 segment comprises an amino acid sequence that is at least 80% identical (e.g., at least 85%, 90%, 95%, 97%, 99%, or 100% identical) to SEQ ID NO: 13, the VH FW3 segment comprises an amino acid sequence that is at least 80% identical (e.g., at least 85%, 90%, 95%, 97%, 99%, or 100% identical) to SEQ ID NO: 14, and the VH FW4 segment comprises an amino acid sequence that is at least 80% identical (e.g., at least 85%, 90%, 95%, 97%, 99%, or 100% identical) to SEQ ID NO: 4.

In some embodiments, the VH FW1 segment comprises the amino acid sequence of SEQ ID NO: 12, the VH FW2 segment comprises the amino acid sequence of SEQ ID NO: 13, the VH FW3 segment comprises the amino acid sequence of SEQ ID NO: 14, and the VH FW4 segment comprises the amino acid sequence of SEQ ID NO: 5.

In some embodiments, the HCDR1 segment comprises from 1 to 25 (e.g., 1, 2, 3, 4, 5, 6, 7, 8, 9, 10, 11, 12, 13, 14, 15, 16, 17, 18, 19, 20, 21, 22, 23, 24, or 25) amino acid residues. In some embodiments, the HCDR1 segment comprises an amino acid sequence that is at least 80% identical (e.g., at least 85%, 90%, 95%, 97%, 99%, or 100% identical) to a sequence selected from the group consisting of SEQ ID NOS: 18-57 set forth in Table 2 below:

TABLE 2
Exemplary HCDR1 of canine antibodies

| | SEQ |
|---|---|
| HCDR1 Sequence | ID NO: |
| GFTFSNYGMT | 18 |
| GFTFSSFGMN | 19 |
| GFTFSNYGMN | 20 |
| GFSFSDSGLN | 21 |
| GFTFSNYNMG | 22 |
| GFTFNNYGMS | 23 |
| GFTFSTYGMT | 24 |
| GFTFSDYNMG | 25 |
| GFTFIRFGMS | 26 |
| GFTFSDYNMA | 27 |
| GFTFSTYGMN | 28 |
| GFTFSDYGMN | 29 |
| GFTFSTYNMG | 30 |
| GFTFSDYGMT | 31 |
| GFTFSNYGLS | 32 |
| GFTFSNSGMS | 33 |
| GFTFSNYPMT | 34 |
| GFTFRSYGMG | 35 |
| GFTFNIYGMS | 36 |
| GFTFSRYNMG | 37 |
| GGSVTSSYYWN | 38 |
| GGSVTSNYYWN | 39 |
| GGSVTSTYYWN | 40 |
| RGSVTSSYYWN | 41 |
| GGSVTNSYYWN | 42 |
| GGSVTSDYYWN | 43 |
| GGSVTSSHYWN | 44 |
| GGSVTSSSYWT | 45 |
| GGSVTSSSYWN | 46 |
| GGSVSSSYYWN | 47 |
| GGSVTSGYYWN | 48 |
| GGSVTTSYYWN | 49 |
| GGSVTNNYYWN | 50 |
| GGSVTNTYYWN | 51 |
| GGSVTTTYYWN | 52 |
| GGSVTSSYYWS | 53 |
| GDSVTSSYYWN | 54 |
| GDSVTSNYYWN | 55 |
| GGSVTSSYHWN | 56 |
| GGSVTSSYYWH | 57 |

In some embodiments, the HCDR1 segment comprises an amino acid sequence selected from the group consisting of SEQ ID NOS: 18-57.

In some embodiments, the HCDR1 segment comprises an amino acid sequence that is at least 80% identical (e.g., at least 85%, 90%, 95%, 97%, 99%, or 100% identical) to a sequence selected from the group consisting of SEQ ID NOS: 18-37. In some embodiments, the HCDR1 segment comprises an amino acid sequence selected from the group consisting of SEQ ID NOS: 18-37.

In some embodiments, the HCDR1 segment comprises an amino acid sequence that is at least 80% identical (e.g., at least 85%, 90%, 95%, 97%, 99%, or 100% identical) to a sequence selected from the group consisting of SEQ ID NOS: 18-37. In some embodiments, the HCDR1 segment comprises an amino acid sequence selected from the group consisting of SEQ ID NOS: 18-37.

In some embodiments, the HCDR1 segment comprises an amino acid sequence that is at least 80% identical (e.g., at least 85%, 90%, 95%, 97%, 99%, or 100% identical) to a sequence selected from the group consisting of SEQ ID NOS: 38-57. In some embodiments, the HCDR1 segment comprises an amino acid sequence selected from the group consisting of SEQ ID NOS: 38-57.

In some embodiments, the HCDR2 segment comprises from 1 to 25 (e.g., 1, 2, 3, 4, 5, 6, 7, 8, 9, 10, 11, 12, 13, 14, 15, 16, 17, 18, 19, 20, 21, 22, 23, 24, or 25) amino acid residues. In some embodiments, the HCDR2 segment comprises an amino acid sequence that is at least 80% identical (e.g., at least 85%, 90%, 95%, 97%, 99%, or 100% identical) to a sequence selected from the group consisting of SEQ ID NOS: 58-97 set forth in Table 3 below:

TABLE 3
Exemplary HCDR2 of canine antibodies

| | SEQ |
|---|---|
| HCDR2 Sequence | ID NO: |
| VANIDSSGTETTYV | 58 |
| VAWIYASGSSTSYA | 59 |
| VAYIKKGGSWTTYA | 60 |
| VTFINSGGSRTTYA | 61 |
| VASINSAGSGTGYA | 62 |
| VAYINSPGNSVDYP | 63 |
| VAYINSGGTNTDYA | 64 |
| VAHIDSGGINTNFA | 65 |
| VAYIDSGGSSTTYS | 66 |
| VAYIDSSGSDTLYA | 67 |
| VANINAGGTSTGYI | 68 |
| VAYIKPGQTFITYL | 69 |
| VAYINSGGTTTHYA | 70 |
| VAYIHSGGTTSTYG | 71 |
| VAYINSGATTTHYA | 72 |
| VAWIYGSGTGTSYG | 73 |
| VAYIDRSGSHTIYA | 74 |
| VAQINIGGSITHYA | 75 |
| VADIDKGGSSTRYA | 76 |
| VAYINSGGNTIGYA | 77 |
| MGYWTGSTNYN | 78 |
| MGYWTGTTNYN | 79 |
| MGYWTGNTNYN | 80 |
| MGYWTGSTKYN | 81 |
| MGYWIGSTSYN | 82 |
| MGYWTGSTSYN | 83 |
| MGYWAGSTNYN | 84 |
| MGYWTGRTNYN | 85 |
| MGYWTGTTKYN | 86 |
| MGYWTGDTNYN | 87 |
| MGYWTGNTKYN | 88 |
| MGYWTGGTNYN | 89 |
| MGYWVGSTTYN | 90 |
| MGYWTGTTRYN | 91 |
| LGYWTGSTNYN | 92 |
| MGYWTGSTDYN | 93 |
| MGFWTGSTNYN | 94 |
| MGYWSGSTNYN | 95 |
| MGYWTGYTNYN | 96 |
| MGYWTGSTTYN | 97 |

In some embodiments, the HCDR2 segment comprises an amino acid sequence selected from the group consisting of SEQ ID NOS: 58-97.

In some embodiments, the HCDR2 segment comprises an amino acid sequence that is at least 80% identical (e.g., at least 85%, 90%, 95%, 97%, 99%, or 100% identical) to a sequence selected from the group consisting of SEQ ID NOS: 58-77. In some embodiments, the HCDR2 segment comprises an amino acid sequence selected from the group consisting of SEQ ID NOS: 58-77.

In some embodiments, the HCDR2 segment comprises an amino acid sequence that is at least 80% identical (e.g., at least 85%, 90%, 95%, 97%, 99%, or 100% identical) to a sequence selected from the group consisting of SEQ ID NOS: 58-77. In some embodiments, the HCDR2 segment comprises an amino acid sequence selected from the group consisting of SEQ ID NOS: 58-77.

In some embodiments, the HCDR2 segment comprises an amino acid sequence that is at least 80% identical (e.g., at least 85%, 90%, 95%, 97%, 99%, or 100% identical) to a sequence selected from the group consisting of SEQ ID NOS: 78-97. In some embodiments, the HCDR2 segment comprises an amino acid sequence selected from the group consisting of SEQ ID NOS: 78-97.

In some embodiments, the HCDR3 segment comprises from 1 to 35 (e.g., 1, 2, 3, 4, 5, 6, 7, 8, 9, 10, 11, 12, 13, 14, 15, 16, 17, 18, 19,20,21,22,23,24,25,26,27,28,29,30,31,32,33,34, or 35) amino acid residues. In some embodiments, the HCDR3 segment comprises an amino acid sequence that is at least 80% identical (e.g., at least 85%, 90%, 95%, 97%, 99%, or 100% identical) to a sequence selected from the group consisting of SEQ ID NOS: 98-137 set forth in Table 4 below:

TABLE 4
Exemplary HCDR3 of canine antibodies

| | SEQ |
|---|---|
| HCDR3 Sequence | ID NO: |
| TSTPIYTDAYVANFDY | 98 |
| ASDGDTFGYILNGFHS | 99 |
| AKEVRDTHGLTFNFDS | 100 |
| AWSDYNSYRLGDYLDH | 101 |
| ASESARVIHAYVWFYN | 102 |
| ARSPLGYYGSFSNFDY | 103 |
| ATVWAPYGYGSHNFDY | 104 |
| ARSPLGYYGSVSNFDY | 105 |
| ARSPPGYYGSVSNFDY | 106 |
| ANYEGRYWETVYNFQY | 107 |
| AKDGYDGDIWIDNFDY | 108 |
| AKDGEVKNNWYNWFQY | 109 |
| ANEGPSYYQGLNWFYY | 110 |
| VRLNLAGWGLLREDNF | 111 |
| ASGIYIWIRSHRNFEY | 112 |
| ATPGLGDNKYWEEVIY | 113 |
| AKAADINLWYGRGFDQ | 114 |
| AGLDGGRFSGGNAFGY | 115 |
| ATTTGYGTYETYNIDY | 116 |
| TTSGRTPSGAKYNFAH | 117 |
| ASLPGSSWSGYNFDY | 118 |
| ARDGGWRYGWLGVARPF | 119 |
| DY | |
| VRNRYYGSSSWDFDY | 120 |
| ASLRRYFGSYYSESDL | 121 |
| ASLPGSSWSHFDY | 122 |
| ARGYDSYHPRY | 123 |
| ARRGNIWANGGLFDY | 124 |
| ARDGSRGIYGSGNFHY | 125 |
| VRRSDIWSSNGHFDY | 126 |
| ARVAIAPWNGFDY | 127 |
| AKAYYGGYDGYNLEY | 128 |
| ARRGPHFGSYYY | 129 |
| ARRGSSWYYTYFDY | 130 |
| ARYIYGSGFDY | 131 |
| ASLPGSSWSGYNFEY | 132 |
| ASTGSIYGYAPFN | 133 |
| ARGSSGNFDY | 134 |
| ASGSDIWSSNGHFDS | 135 |
| ARALEGSSWYSTVNFDY | 136 |
| ARGRRGYIYGYADGHFDF | 137 |

In some embodiments, the HCDR3 segment comprises an amino acid sequence selected from the group consisting of SEQ ID NOS: 98-137.

In some embodiments, the HCDR3 segment comprises an amino acid sequence that is at least 80% identical (e.g., at least 85%, 90%, 95%, 97%, 99%, or 100% identical) to a sequence selected from the group consisting of SEQ ID NOS: 98-117. In some embodiments, the HCDR3 segment comprises an amino acid sequence selected from the group consisting of SEQ ID NOS: 98-117.

In some embodiments, the HCDR3 segment comprises an amino acid sequence that is at least 80% identical (e.g., at least 85%, 90%, 95%, 97%, 99%, or 100% identical) to a sequence selected from the group consisting of SEQ ID NOS: 98-117. In some embodiments, the HCDR3 segment comprises an amino acid sequence selected from the group consisting of SEQ ID NOS: 98-117.

In some embodiments, the HCDR3 segment comprises an amino acid sequence that is at least 80% identical (e.g., at least 85%, 90%, 95%, 97%, 99%, or 100% identical) to a sequence selected from the group consisting of SEQ ID NOS: 118-137. In some embodiments, the HCDR3 segment comprises an amino acid sequence selected from the group consisting of SEQ ID NOS: 118-137.

In some embodiments, the HCDR1 segment comprises an amino acid sequence selected from the group consisting of SEQ ID NOS: 18-37, the HCDR2 segment comprises an amino acid sequence selected from the group consisting of SEQ ID NOS: 58-77, and the HCDR3 segment comprises an amino acid sequence selected from the group consisting of SEQ ID NOS: 98-117.

In some embodiments, the HCDR1 segment comprises an amino acid sequence selected from the group consisting of SEQ ID NOS: 18-37, the HCDR2 segment comprises an amino acid sequence selected from the group consisting of SEQ ID NOS: 58-77, and the HCDR3 segment comprises an amino acid sequence selected from the group consisting of SEQ ID NOS: 98-117.

In some embodiments, the HCDR1 segment comprises an amino acid sequence selected from the group consisting of SEQ ID NOS: 38-57, the HCDR2 segment comprises an amino acid sequence selected from the group consisting of SEQ ID NOS: 78-97, and the HCDR3 segment comprises an amino acid sequence selected from the group consisting of SEQ ID NOS: 118-137.

In some embodiments, the polypeptide library of the disclosure comprises VL polypeptide members comprising VL FW1-LCDR1-VL FW2-LCDR2-VL FW3-LCDR3-VL FW4 segments.

In some embodiments, the VL FW1 segment comprises an amino acid sequence that is at least 80% identical (e.g., at least 85%, 90%, 95%, 97%, 99%, or 100% identical) to a sequence selected from the group consisting of:

(a)
(SEQ ID NO: 5)
QSVLTQPTSVSGSLGQRVTISCSGS;
(b)
(SEQ ID NO: 9)
QAVLTQPPSVSAALGQRVTISCTGS;
and
(c)
(SEQ ID NO: 15)
QSMLTQPASVSGSLGQKVTISCTGS.

In some embodiments, the VL FW1 segment comprises the amino acid sequence of SEQ ID NO: 5. In some embodiments, the VL FW1 segment comprises the amino acid sequence of SEQ ID NO: 9. In some embodiments, the VL FW1 segment comprises the amino acid sequence of SEQ ID NO: 15.

In some embodiments, the VL FW2 segment comprises an amino acid sequence that is at least 80% identical (e.g., at least 85%, 90%, 95%, 97%, 99%, or 100% identical) to a sequence selected from the group consisting of:

(a)
(SEQ ID NO: 6)
WYQQLPGKAPKLLV;
(b)
(SEQ ID NO: 10)
WYQQLPGKSPKTII;
and
(c)
(SEQ ID NO: 16)
WYQQLPGIGPRTVI.

In some embodiments, the VL FW2 segment comprises the amino acid sequence of SEQ ID NO: 6. In some embodiments, the VL FW2 segment comprises the amino acid sequence of SEQ ID NO: 10. In some embodiments, the VL FW2 segment comprises the amino acid sequence of SEQ ID NO: 16.

In some embodiments, the VL FW3 segment comprises an amino acid sequence that is at least 80% identical (e.g., at least 85%, 90%, 95%, 97%, 99%, or 100% identical) to a sequence selected from the group consisting of:

(a)
(SEQ ID NO: 7)
GVPDRFSGSKSGNSATLTITGLQAEDEADYYC;
(b)
(SEQ ID NO: 11)
GVPVRFSGSKSGSTATLTITGIQAEDEADYYC;
and
(c)
(SEQ ID NO: 17)
GVPDRFSGSKSGSSATLTISGLQAEDEAEYYC.

In some embodiments, the VL FW3 segment comprises the amino acid sequence of SEQ ID NO: 7. In some embodiments, the VL FW3 segment comprises the amino acid sequence of SEQ ID NO: 11. In some embodiments, the VL FW3 segment comprises the amino acid sequence of SEQ ID NO: 17.

In some embodiments, the VL FW4 segment comprises an amino acid sequence that is at least 80% identical (e.g., at least 85%, 90%, 95%, 97%, 99%, or 100% identical) to the sequence of FGGGTHLTVL (SEQ ID NO: 8).

In some embodiments, the VL FW4 segment comprises the amino acid sequence of SEQ ID NO: 8.

In some embodiments, the VL FW1 segment comprises an amino acid sequence that is at least 80% identical (e.g., at least 85%, 90%, 95%, 97%, 99%, or 100% identical) to SEQ ID NO: 5, the VL FW2 segment comprises an amino acid sequence that is at least 80% identical (e.g., at least 85%, 90%, 95%, 97%, 99%, or 100% identical) to SEQ ID NO: 6, the VL FW3 segment comprises an amino acid sequence that is at least 80% identical (e.g., at least 85%, 90%, 95%, 97%, 99%, or 100% identical) to SEQ ID NO: 7, and the VL FW4 segment comprises an amino acid sequence that is at least 80% identical (e.g., at least 85%, 90%, 95%, 97%, 99%, or 100% identical) to SEQ ID NO: 8.

In some embodiments, the VL FW1 segment comprises the amino acid sequence of SEQ ID NO: 5, the VL FW2 segment comprises the amino acid sequence of SEQ ID NO: 6, the VL FW3 segment comprises the amino acid sequence of SEQ ID NO: 7, and the VL FW4 segment comprises the amino acid sequence of SEQ ID NO: 8.

In some embodiments, the VL FW1 segment comprises an amino acid sequence that is at least 80% identical (e.g., at least 85%, 90%, 95%, 97%, 99%, or 100% identical) to SEQ ID NO: 9, the VL FW2 segment comprises an amino acid sequence that is at least 80% identical (e.g., at least 85%, 90%, 95%, 97%, 99%, or 100% identical) to SEQ ID NO: 10, the VL FW3 segment comprises an amino acid sequence that is at least 80% identical (e.g., at least 85%, 90%, 95%, 97%, 99%, or 100% identical) to SEQ ID NO: 11, and the VL FW4 segment comprises an amino acid sequence that is at least 80% identical (e.g., at least 85%, 90%, 95%, 97%, 99%, or 100% identical) to SEQ ID NO: 8.

In some embodiments, the VL FW1 segment comprises the amino acid sequence of SEQ ID NO: 9, the VL FW2 segment comprises the amino acid sequence of SEQ ID NO: 10, the VL FW3 segment comprises the amino acid sequence of SEQ ID NO: 11, and the VL FW4 segment comprises the amino acid sequence of SEQ ID NO: 8.

In some embodiments, the VL FW1 segment comprises an amino acid sequence that is at least 80% identical (e.g., at least 85%, 90%, 95%, 97%, 99%, or 100% identical) to SEQ ID NO: 15, the VL FW2 segment comprises an amino acid sequence that is at least 80% identical (e.g., at least 85%, 90%, 95%, 97%, 99%, or 100% identical) to SEQ ID NO: 16, the VL FW3 segment comprises an amino acid sequence that is at least 80% identical (e.g., at least 85%, 90%, 95%, 97%, 99%, or 100% identical) to SEQ ID NO: 17, and the VL FW4 segment comprises an amino acid sequence that is at least 80% identical (e.g., at least 85%, 90%, 95%, 97%, 99%, or 100% identical) to SEQ ID NO: 8.

In some embodiments, the VL FW1 segment comprises the amino acid sequence of SEQ ID NO: 15, the VL FW2 segment comprises the amino acid sequence of SEQ ID NO: 16, the VL FW3 segment comprises the amino acid sequence of SEQ ID NO: 17, and the VL FW4 segment comprises the amino acid sequence of SEQ ID NO: 8.

In some embodiments, the LCDR1 segment comprises from 1 to25 (e.g., 1, 2, 3, 4, 5, 6, 7, 8, 9, 10, 11, 12, 13, 14, 15, 16, 17, 18, 19, 20, 21, 22, 23, 24, or 25) amino acid residues. In some embodiments, the LCDR1 segment comprises an amino acid sequence that is at least 80% identical (e.g., at least 85%, 90%, 95%, 97%, 99%, or 100% identical) to a sequence selected from the group consisting of SEQ ID NOS: 138-197 set forth in Table 5 below:

TABLE 5
Exemplary LCDR1 of canine antibodies

| LCDR1 | SEQ |
|---|---|
| Sequence | ID NO: |
| TNNIGIVGAS | 138 |
| TNNIGIVGAN | 139 |
| TNNIGLVGAS | 140 |
| TNNIGIVGAT | 141 |
| TGNIGVVGAT | 142 |
| TNNIGIAGAS | 143 |
| TDNIGILGAT | 144 |
| TNNIGTVGAS | 145 |
| TNNIGVVGAS | 146 |
| TNNIGIVGAA | 147 |
| TNNIGTVGAT | 148 |
| ANNIGIFGAA | 149 |
| TDDIGLIGAT | 150 |
| TNNIGILGAT | 151 |
| TDNIGTIGAR | 152 |
| ANNIGIVGAT | 153 |
| TNDIRIVGAS | 154 |
| TNNINIVGAS | 155 |
| TNDIGIVGAT | 156 |
| TDNVGIVGAN | 157 |
| NTNIGSGYDVQ | 158 |
| DTNIGSGYDVQ | 159 |
| NTNIGSAYDVQ | 160 |
| NTNIGRGYDVQ | 161 |
| NTNIGRGHDVQ | 162 |
| NTNIGGGYDVQ | 163 |
| NTNIGSGYGVQ | 164 |
| NTNIGSNYDVQ | 165 |
| NTNIGSDYDVQ | 166 |
| NTNIGKDYDVQ | 167 |
| NTNIGSVYDVQ | 168 |
| NTNIGSGYDVH | 169 |
| NTNIGSGFDVQ | 170 |
| DTNIGGDYDVQ | 171 |
| DTNIGSNYDVQ | 172 |
| DTNIGTGYDVQ | 173 |
| NTNIGKNYDVH | 174 |
| ETNIGKNYDVQ | 175 |
| NTNIGSYFDVQ | 176 |
| SSNIGGNYVG | 178 |
| SSNIGGNYVA | 179 |
| SSNIGDNYVG | 180 |
| SSNIGGNNVG | 181 |
| NSNIGGNYVG | 182 |
| RSNIGGNYVG | 183 |
| SSNIGGNFVG | 184 |
| SSNIGDNYVA | 185 |
| SSNIGANYVA | 186 |
| SSNIGGNSVG | 187 |
| NSNIGGNYVA | 188 |
| SSNIGNNYVG | 189 |
| GSNIRTNYVG | 190 |
| SSNIGGYYVG | 191 |
| SSNIGGNHVG | 192 |
| SSNIGANYVG | 193 |
| SSNIGGNDVG | 194 |
| SSNIGSNYVG | 195 |
| SSNIGTNYVG | 196 |
| GSNIGGNYVG | 197 |

In some embodiments, the LCDR1 segment comprises an amino acid sequence selected from the group consisting of SEQ ID NOS: 138-197.

In some embodiments, the LCDR1 segment comprises an amino acid sequence that is at least 80% identical (e.g., at least 85%, 90%, 95%, 97%, 99%, or 100% identical) to a sequence selected from the group consisting of SEQ ID NOS: 138-157. In some embodiments, the LCDR1 segment comprises an amino acid sequence selected from the group consisting of SEQ ID NOS: 138-157.

In some embodiments, the LCDR1 segment comprises an amino acid sequence that is at least 80% identical (e.g., at least 85%, 90%, 95%, 97%, 99%, or 100% identical) to a sequence selected from the group consisting of SEQ ID NOS: 158-177. In some embodiments, the LCDR segment comprises an amino acid sequence selected from the group consisting of SEQ ID NOS: 158-177.

In some embodiments, the LCDR1 segment comprises an amino acid sequence that is at least 80% identical (e.g., at least 85%, 90%, 95%, 97%, 99%, or 100% identical) to a sequence selected from the group consisting of SEQ ID NOS: 178-197. In some embodiments, the LCDR1 segment comprises an amino acid sequence selected from the group consisting of SEQ ID NOS: 178-197.

In some embodiments, the LCDR2 segment comprises from 1 to 25 (e.g., 1, 2, 3, 4, 5, 6, 7, 8, 9, 10,11,12,13,14,15,16,17,18,19, 20, 21, 22, 23, 24, or 25) amino acid residues. In some embodiments, the LCDR2 segment comprises an amino acid sequence that is at least 80% identical (e.g., at least 85%, 90%, 95%, 97%, 99%, or 100% identical) to a sequence selected from the group consisting of SEQ ID NOS: 198-257 set forth in Table 6 below:

TABLE 6
Exemplary LCDR2 of canine antibodies

| LCDR2 | SEQ |
|---|---|
| Sequence | ID NO: |
| DSDGDRPS | 198 |
| YSDGDRPS | 199 |
| YSDGNRPS | 200 |
| YSDGSRPS | 201 |
| YNDGNRPS | 202 |
| DNDGDRPS | 203 |
| YSNGDRPS | 204 |
| YDDGDRPS | 205 |
| YTNGDRPS | 206 |
| YSDGKRPS | 207 |
| HSDGDRPS | 208 |
| DIDGDRPS | 209 |
| YRNGDRPS | 210 |
| FSDGDRPS | 211 |
| HSDGERPS | 212 |
| DSDGNRPS | 213 |
| DSDGERPS | 214 |
| YNNGDRPS | 215 |
| DGDGDRPS | 216 |
| YTTGDRPS | 217 |
| YGNSNRPS | 218 |
| YGNTNRPS | 219 |
| YGNNNRPS | 220 |
| YGDSNRPS | 221 |
| YGNRNRPS | 222 |
| YGNINRPS | 223 |
| YGYTNRPS | 224 |
| YGNSYRPS | 225 |
| YGNGNRPS | 226 |
| FGNSNRPS | 227 |
| YGNSDRPS | 228 |
| FGNTNRPS | 229 |
| YGTTFRPS | 230 |
| YANSNRPS | 231 |
| HGDSNRPS | 232 |
| YGDNNRPS | 233 |
| YGDTNRPS | 234 |
| YDNTNRPS | 235 |
| YGNDNRPS | 236 |
| YGNNYRPS | 238 |
| YGNDYRPS | 239 |
| YGDNYRPS | 240 |
| YANNYRPS | 241 |
| YGNGFRPP | 242 |
| YGNDHRPS | 243 |
| YSNNYRPS | 244 |
| YGDDYRPS | 245 |
| YGNSYRPS | 246 |
| YGNNHRPS | 247 |
| YGNYYRPS | 248 |
| YRDTYRPS | 249 |
| YGNNNRPS | 250 |
| YGDDHRPS | 251 |
| YGNSHRPS | 252 |
| YGDNHRPS | 253 |
| YGNTYRPS | 254 |
| YGYNYRPS | 255 |
| FGNNYRPS | 256 |
| YDNNYRPS | 257 |

In some embodiments, the LCDR2 segment comprises an amino acid sequence selected from the group consisting of SEQ ID NOS: 198-257.

In some embodiments, the LCDR2 segment comprises an amino acid sequence that is at least 80% identical (e.g., at least 85%, 90%, 95%, 97%, 99%, or 100% identical) to a sequence selected from the group consisting of SEQ ID NOS: 198-217. In some embodiments, the LCDR2 segment comprises an amino acid sequence selected from the group consisting of SEQ ID NOS: 198-217.

In some embodiments, the LCDR2 segment comprises an amino acid sequence that is at least 80% identical (e.g., at least 85%, 90%, 95%, 97%, 99%, or 100% identical) to a sequence selected from the group consisting of SEQ ID NOS: 218-237. In some embodiments, the LCDR2 segment comprises an amino acid sequence selected from the group consisting of SEQ ID NOS: 218-237.

In some embodiments, the LCDR2 segment comprises an amino acid sequence that is at least 80% identical (e.g., at least 85%, 90%, 95%, 97%, 99%, or 100% identical) to a sequence selected from the group consisting of SEQ ID NOS: 238-257. In some embodiments, the LCDR2 segment comprises an amino acid sequence selected from the group consisting of SEQ ID NOS: 238-257.

In some embodiments, the LCDR3 segment comprises from 1 to 35 (e.g., 1, 2, 3, 4, 5, 6, 7, 8, 9, 10, 11, 12, 13, 14, 15, 16, 17, 18, 19,20,21,22,23,24,25,26,27,28,29,30,31,32,33,34, or35) amino acid residues. In some embodiments, the LCDR3 segment comprises an amino acid sequence that is at least 80% identical (e.g., at least 85%, 90%, 95%, 97%, 99%, or 100% identical) to a sequence selected from the group consisting of SEQ ID NOS: 258-317 set forth in Table 7 below:

TABLE 7
Exemplary LCDR3 of canine antibodies

| LCDR3 | SEQ ID |
|---|---|
| Sequence | NO: |
| QSFDTTLDAAV | 258 |
| QSFDTTLDAYV | 259 |
| QSFDTTLDAGV | 260 |
| QSFDTTLDAYAV | 261 |
| QSFDTTLDAHV | 262 |
| QSFDTTLDAHAV | 263 |
| QSFDTTLDAV | 264 |
| QSFDTTLDAPV | 265 |
| QSFDIALQAHV | 266 |
| QSFDTTLDASV | 267 |
| QSFDTTLDAIV | 268 |
| QSFDTTLDGAV | 269 |
| QSFDNTLDIVL | 270 |
| QSVDSTLGAAV | 271 |
| QSFDTTLDDHVV | 272 |
| QSFDATLNAPV | 273 |
| QSYETTLDVV | 274 |
| QSFDTTLIV | 275 |
| QSFDTTLDAHIV | 276 |
| QSFDTTLGAAV | 277 |
| QSYDDNLDGHAV | 278 |
| QSYDDNLDGAV | 279 |
| QSYDDNLDAV | 280 |
| QSYDDNLDGHV | 281 |
| QSYDDNLDGYV | 282 |
| QSYDDNLDGIV | 283 |
| QSYDDNLDGV | 284 |
| QSYDDNLDGHYV | 285 |
| QSYDDKFDGHAV | 286 |
| QSYDDNFDGAV | 287 |
| QSYDDNLDGLV | 288 |
| QSYDDNLDGHAA | 289 |
| QSYDDNLDGHIV | 290 |
| QSYDGDLDGHV | 291 |
| QSYDDDLDGAV | 292 |
| QSYDDNLGGAV | 293 |
| QSYDDNLDDAV | 294 |
| QSYDDNLDYV | 295 |
| QSYDDNLDGHVV | 296 |
| QSYDDNLDGGV | 297 |
| SSWDDSLRGHV | 298 |
| SSWDDSLRGAV | 299 |
| SSWDDSLRGYV | 300 |
| SSWDDSLRGIV | 301 |
| SSWDDSLRGVV | 302 |
| SSWDDSLRGGV | 303 |
| SSWDDTLRGHV | 304 |
| SSWDDSLQYI | 305 |
| SSWDDGLRGHV | 306 |
| SSWDDSLRGPV | 307 |
| SSWDDSLRGTV | 308 |
| SSWDDTLSGHV | 309 |
| SSWDNSVRGYV | 310 |
| SSWDDSLRGSV | 311 |
| SSWDDRLRGAV | 312 |
| SSWDDSLRGHL | 313 |
| SSWDDSLWTPV | 314 |
| SSWDDSVRGVL | 315 |
| SSWDDRVRGVL | 316 |
| ASWDDNLRVQV | 317 |

In some embodiments, the LCDR3 segment comprises an amino acid sequence selected from the group consisting of SEQ ID NOS: 258-317.

In some embodiments, the LCDR3 segment comprises an amino acid sequence that is at least 80% identical (e.g., at least 85%, 90%, 95%, 97%, 99%, or 100% identical) to a sequence selected from the group consisting of SEQ ID NOS: 258-277. In some embodiments, the LCDR3 segment comprises an amino acid sequence selected from the group consisting of SEQ ID NOS: 258-277.

In some embodiments, the LCDR3 segment comprises an amino acid sequence that is at least 80% identical (e.g., at least 85%, 90%, 95%, 97%, 99%, or 100% identical) to a sequence selected from the group consisting of SEQ ID NOS: 278-297. In some embodiments, the LCDR3 segment comprises an amino acid sequence selected from the group consisting of SEQ ID NOS: 278-297.

In some embodiments, the LCDR3 segment comprises an amino acid sequence that is at least 80% identical (e.g., at least 85%, 90%, 95%, 97%, 99%, or 100% identical) to a sequence selected from the group consisting of SEQ ID NOS: 298-317. In some embodiments, the LCDR3 segment comprises an amino acid sequence selected from the group consisting of SEQ ID NOS: 298-317.

In some embodiments, the LCDR1 segment comprises an amino acid sequence selected from the group consisting of SEQ ID NOS: 138-157, the LCDR2 segment comprises an amino acid sequence selected from the group consisting of SEQ ID NOS: 198-217, and the LCDR3 segment comprises an amino acid sequence selected from the group consisting of SEQ ID NOS: 258-277.

In some embodiments, the LCDR1 segment comprises an amino acid sequence selected from the group consisting of SEQ ID NOS: 158-177, the LCDR2 segment comprises an amino acid sequence selected from the group consisting of SEQ ID NOS: 218-237, and the LCDR3 segment comprises an amino acid sequence selected from the group consisting of SEQ ID NOS: 278-297.

In some embodiments, the LCDR1 segment comprises an amino acid sequence selected from the group consisting of SEQ ID NOS: 178-197, the LCDR2 segment comprises an amino acid sequence selected from the group consisting of SEQ ID NOS: 238-257, and the LCDR3 segment comprises an amino acid sequence selected from the group consisting of SEQ ID NOS: 298-317.

### Polypeptides

In some embodiments, the VH polypeptide members are connected to the VL polypeptide members by a linker. In some embodiments, the linker is made up of from 1 to 20 amino acids linked by peptide bonds, wherein the amino acids are selected from the 20 naturally occurring amino acids. Some of these amino acids may be glycosylated, as is well understood by those in the art. In some embodiments, the 1 to 20 amino acids are selected from glycine, alanine, proline, asparagine, glutamine, and lysine. In some embodiments, a linker is made up of a majority of amino acids that are sterically unhindered, such as glycine and alanine. Examples of linkers include: G; S; GS; GGS; SGG; GGGS (SEQ ID NO: 323); SGGG (SEQ ID NO: 324); GGGGS (SEQ ID NO: 325); SGGGG (SEQ ID NO: 326); GGGGGS (SEQ ID NO: 327); SGGGGG (SEQ ID NO: 328); GGGGGGS (SEQ ID NO: 329); SGGGGGG (SEQ ID NO: 330); (GGGGS)ₙ (SEQ ID NO: 325), wherein n is an integer of one or more (e.g., 1, 2, 3, 4, 5); and (SGGGG)ₙ (SEQ ID NO: 326), wherein n is an integer of one or more (e.g., 1, 2, 3, 4, 5). In some embodiments, the linker comprises the amino acid sequence of GGGGSGGGGSGGGGS (SEQ ID NO: 331).

The polypeptide library of the disclosure can be used to generate polypeptides with desirable properties, such as binding to a particular antigen. For example, the polypeptide library may comprise one or more antibodies or antigen-binding fragments thereof.

In some embodiments, the antibody or antigen-binding fragment thereof is selected from the group consisting of a monoclonal antibody or antigen-binding fragment thereof, a polyclonal antibody or antigen-binding fragment thereof, a canine antibody or antigen-binding fragment thereof, a caninized antibody or antigen-binding fragment thereof, a bispecific antibody or antigen-binding fragment thereof, a multi-specific antibody or antigen-binding fragment thereof, a dual-variable immunoglobulin domain, a monovalent antibody or antigen-binding fragment thereof, a chimeric antibody or antigen-binding fragment thereof, a single-chain Fv molecule (scFv), a diabody, a triabody, a Fv fragment, a Fab fragment, a F(ab′)2 molecule, and a tandem scFv (taFv).

### Nucleic Acids, Vectors, and Host Cells

The polypeptide library of the disclosure can be encoded by one or more nucleic acids. For instance, to express polypeptides of the polypeptide library of the disclosure, a host cell can be transfected with one or more recombinant expression vectors carrying DNA fragments encoding members of the polypeptide library, such that polypeptide members are expressed in the host cell and, optionally, secreted into the medium in which the host cells are cultured, from which medium the polypeptides can be recovered. Standard recombinant DNA methodologies are used to obtain nucleic acids encoding members of the polypeptide library, incorporate these nucleic acids into recombinant expression vectors and introduce the vectors into host cells that are known in the art.

Viral genomes provide a rich source of vectors that can be used for the efficient delivery of exogenous genes into the genome of a cell (e.g., a eukaryotic or prokaryotic cell). Viral genomes are particularly useful vectors for gene delivery because the polynucleotides contained within such genomes are typically incorporated into the genome of a target cell by generalized or specialized transduction. These processes occur as part of the natural viral replication cycle, and do not require added proteins or reagents to induce gene integration. Examples of viral vectors include a retrovirus, adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses useful for delivering polynucleotides encoding antibody light and heavy chains or antibody fragments of the disclosure include Norwalk virus, togavirus, flavivirus, reoviruses, papovavirus, hepadnavirus, and hepatitis virus. Examples of retroviruses include avian leukosis-sarcoma, mammalian C-type, B-type viruses, D-type viruses, HTLV-BLV group, lentivirus, and spumavirus. Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason-Pfizer monkey virus, simian immunodeficiency virus, simian sarcoma virus, Rous sarcoma virus and lentiviruses.

It is possible to express the polypeptide library of the disclosure in either prokaryotic or eukaryotic host cells. In some embodiments, expression of the polypeptide library is performed in eukaryotic cells, e.g., mammalian host cells, for optimal secretion of a properly folded and immunologically active antibody. Exemplary mammalian host cells for expressing recombinant polypeptides (e.g., members of the polypeptide library of the disclosure) include Chinese Hamster Ovary (CHO) cells, NSO myeloma cells, COS cells, 293 cells, and SP2/0 cells. Additional cell types that may be useful for the expression of the polypeptide library of the disclosure include bacterial cells, such as BL-21(DE3) E. colicells, which can be transformed with vectors containing foreign DNA according to established protocols known in the art. Additional eukaryotic cells that may be useful for expression of polypeptides include yeast cells, such as auxotrophic strains of S. cerevisiae, which can be transformed and selectively grown in incomplete media according to established procedures known in the art. When recombinant expression vectors containing genes encoding one or more members of the polypeptide library are introduced into mammalian host cells, polypeptides are produced by culturing the host cells for a period of time sufficient to allow for expression of the antibody in the host cells or secretion of the polypeptides into the culture medium in which the host cells are grown. Members of the polypeptide library of the disclosure can be recovered from the culture medium using standard protein purification methods. Once members of the polypeptide library of the disclosure has been produced by recombinant expression, they can be purified by any method known in the art, such as a method useful for purification of an immunoglobulin molecule, for example, by chromatography (e.g., ion exchange, affinity, and sizing column chromatography), centrifugation, differential solubility, or by any other standard technique for the purification of proteins.

Further, members of polypeptide library of the disclosure can be fused to heterologous polypeptide sequences described herein or otherwise known in the art to facilitate purification or to produce therapeutic conjugates.

Selection from Library

To select members (e.g., antibodies or antigen-binding fragments thereof) from the polypeptide library, display methods, such as display on phage, E. coli, or yeast, can be used. In some embodiments, antibodies or antigen-binding fragments thereof are presented as a fusion polypeptide on a bacteriophage surface protein. In some embodiments, antibodies or antigen-binding fragments thereof are selected by affinity to a desired antigen molecule. For example, the antibody-displaying phage particles are contacted with an antigen molecule that is immobilized onto a solid phase (e.g., assay plate or beads) or in solution. Phages expressing antibodies having desired affinity to the target antigen molecule are selected, while phages expressing antibodies that do not bind to the target antigen molecule are washed away. In phage display, the antibody presented on the selected phages corresponds one-to-one to the nucleic acid coding the same, and therefore, the antibody of interest can be easily identified. Further, the nucleic caid encoding the antibody can be easily amplified and purified. Therefore, phage display is widely used as method for screening and isolating antibodies from large libraries.

Desirable antigen molecules include, but are not limited to, antigen molecules that are related to a canine disease or disorder. In some embodiments, the antigen molecule is selected from the group consisting of: 17-IA, 4-1BB, 4Dc, 6-keto-PGF1a, 8-iso-PGF2a, 8-oxo-dG, Al Adenosine Receptor, A33, ACE, ACE-2, Activin, Activin A, Activin AB, Activin B, Activin C, Activin RIA, Activin RIA ALK-2, Activin RIB ALK-4, Activin RIIA, Activin RIIB, ADAM, ADAM10, ADAM12, ADAM15, ADAM17/TACE, ADAMS, ADAM9, ADAMTS, ADAMTS4, ADAMTS5, Addressins, aFGF, ALCAM, ALK, ALK-1, ALK-7, alpha-1-antitrypsin, alpha-V/beta-1 antagonist, ANG, Ang, APAF-1, APE, APJ, APP, APRIL, AR, IgE, Angiotensin type 1 (AT1) receptor, Angiotensin type 2 (AT2) receptor, ARC, ART, Artemin, anti-Id, ASPARTIC, Atrial natriuretic factor, av/b3 integrin, Axl, b2M, B7-1, B7-2, B7-H, B-lymphocyte Stimulator (BlyS), BACE, BACE-1, Bad, BAFF, BAFF-R, Bag-1, BAK, Bax, BCA-1, BCAM, Bcl, BCMA, BDNF, b-ECGF, bFGF, BID, Bik, BIM, BLC, BL-CAM, BLK, BMP, BMP-2 BMP-2a, BMP-3 Osteogenin, BMP-4 BMP-2b, BMP-5, BMP-6 Vgr-1, BMP-7 (OP-1), BMP-8 (BMP-8a, OP-2), BMPR, BMPR-IA (ALK-3), BMPR-IB (ALK-6), BRK-2, RPK-1, BMPR-II (BRK-3), BMPs, b-NGF, BOK, Bombesin, Bone-derived neurotrophic factor, BPDE, BPDE-DNA, BTC, complement factor 3 (C3), C3a, C4, C5, C5a, C10, CA125, CAD-8, Calcitonin, cAMP, carcinoembryonic antigen (CEA), carcinoma-associated antigen, Cathepsin A, Cathepsin B, Cathepsin C/DPPI, Cathepsin D, Cathepsin E, Cathepsin H, Cathepsin L, Cathepsin O, Cathepsin S, Cathepsin V, Cathepsin X/Z/P, CBL, CC1, CCK2, CCL, CCL1, CCL11, CCL12, CCL13, CCL14, CCL15, CCL16, CCL17, CCL18, CCL19, CCL2, CCL20, CCL21, CCL22, CCL23, CCL24, CCL25, CCL26, CCL27, CCL28, CCL3, CCL4, CCL5, CCL6, CCL7, CCL8, CCL9/10, CCR, CCR1, CCR10, CCR10, CCR2, CCR3, CCR4, CCR5, CCR6, CCR7, CCR8, CCR9, CD1, CD2, CD3, CD3E, CD4, CD5, CD6, CD7, CD8, CD10, CD11a, CD11b, CD11c, CD13, CD14, CD15, CD16, CD18, CD19, CD20, CD21, CD22, CD23, CD25, CD27L, CD28, CD29, CD30, CD30L, CD32, CD33 (p67 proteins), CD34, CD38, CD40, CD40L, CD44, CD45, CD46, CD47, CD49a, CD52, CD54, CD55, CD56, CD61, CD64, CD66e, CD74, CD80 (B7-1), CD89, CD95, CD123, CD137, CD138, CD140a, CD146, CD147, CD148, CD152, CD164, CEACAM5, CFTR, cGMP, CINC, Clostridium botulinum toxin, Clostridium perfringens toxin, CKb8-1, CLC, CMV, CMV UL, CNTF, CNTN-1, COX, C-Ret, CRG-2, CT-1, CTACK, CTGF, CTLA-4, CX3CL1, CX3CR1, CXCL, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCR, CXCR1, CXCR2, CXCR3, CXCR4, CXCR5, CXCR6, cytokeratin tumor-associated antigen, DAN, DCC, DcR3, DC-SIGN, Decay accelerating factor, des(1-3)-IGF-I (brain IGF-1), Dhh, digoxin, DNAM-1, Dnase, Dpp, DPPIV/CD26, Dtk, ECAD, EDA, EDA-A1, EDA-A2, EDAR, EGF, EGFR (ErbB-1), EMA, EMMPRIN, ENA, endothelin receptor, Enkephalinase, eNOS, Eot, eotaxin1, EpCAM, Ephrin B2/EphB4, EPO, ERCC, E-selectin, ET-1, Factor Ila, Factor VII, Factor VilIc, Factor IX, fibroblast activation protein (FAP), Fas, FcR1, FEN-1, Ferritin, FGF, FGF-19, FGF-2, FGF3, FGF-8, FGFR, FGFR-3, Fibrin, FL, FLIP, Flt-3, Flt-4, Follicle stimulating hormone, Fractalkine, FZD1, FZD2, FZD3, FZD4, FZD5, FZD6, FZD7, FZD8, FZD9, FZD10, G250, Gas 6, GCP-2, GCSF, GD2, GD3, GDF, GDF-1, GDF-3 (Vgr-2), GDF-5 (BMP-14, CDMP-1), GDF-6 (BMP-13, CDMP-2), GDF-7 (BMP-12, CDMP-3), GDF-8 (Myostatin), GDF-9, GDF-15 (MIC-1), GDNF, GDNF, GFAP, GFRa-1, GFR-alphai, GFR-alpha2, GFR-alpha3, GITR, GLP1, GLP2, Glucagon, Glut 4, glycoprotein lb/IIla (GP IIb/IIla), GM-CSF, gp130, gp72, GRO, GnRH, Growth hormone releasing factor, Hapten (NP-cap or NIP-cap), HB-EGF, HCC, HCMV gB envelope glycoprotein, HCMV) gH envelope glycoprotein, HCMV UL, Hemopoietic growth factor (HGF), Hep B gp120, heparanase, Her2, Her2/neu (ErbB-2), Her3 (ErbB-3), Her4 (ErbB-4), herpes simplex virus (HSV) gB glycoprotein, HSV gD glycoprotein, HGFA, High molecular weight melanoma-associated antigen (HMW-MAA), HIV gp120, HIV IIIB gp120 V3 loop, HLA, HLA-DR, HM1.24, HMFG PEM, HRG, Hrk, cardiac myosin, cytomegalovirus (CMV), growth hormone (GH), HVEM, 1-309, IAP, ICAM, ICAM-1, ICAM-3, ICE, ICOS, IFNg, Ig, IgA receptor, IgE, IGF, IGF binding proteins, IGF-1R, IGFBP, IGF-I, IGF-II, IL, IL-1, IL-1R, IL-2, IL-2R, IL-4, IL-4R, IL-5, IL-5R, IL-6, IL-6R, IL-8, IL-9, IL-10, IL-12, IL-13, IL-15, IL-17, IL-18, IL-18R, IL-21, IL-22, IL-23, IL-25, IL-31, IL-33, interleukin receptor (e.g., IL-1R, IL-2R, IL-4R, IL-5R, IL-6R, IL-8R, IL-9R, IL-10R, IL-12R, IL-13R, IL-15R, IL-17R, IL-18R, IL-21R, IL-22R, IL-23R, IL-25R, IL-31R, IL-33R), interferon (INF)-alpha, INF-beta, INF-gamma, Inhibin, iNOS, Insulin A-chain, Insulin B-chain, Insulin-like growth factor 1, integrin alpha2, integrin alpha3, integrin alpha4, integrin alpha4/betai, integrin alpha4/beta7, integrin alpha5 (alphaV), integrin alpha5/betai, integrin alpha5/beta3, integrin alpha6, integrin betai, integrin beta2, interferon gamma, IP-10, I-TAC, JE, Kallikrein 2, Kallikrein 5, Kallikrein 6, Kallikrein 11, Kallikrein 12, Kallikrein 14, Kallikrein 15, Kallikrein L1, Kallikrein L2, Kallikrein L3, Kallikrein L4, KC, KDR, Keratinocyte Growth Factor (KGF), laminin 5, LAMP, LAP, LAP (TGF-1), Latent TGF-1, Latent TGF-1 bpi, LBP, LDGF, LECT2, Lefty, Lewis-Y antigen, Lewis-Y related antigen, LFA-1, LFA-3, Lfo, LIF, LIGHT, lipoproteins, LIX, LKN, Lptn, L-Selectin, LT-a, LT-b, LTB4, LTBP-1, Lung surfactant, Luteinizing hormone, Lymphotoxin Beta Receptor, Mac-1, MAdCAM, MAG, MAP2, MARC, MCAM, MCAM, MCK-2, MCP, M-CSF, MDC, Mer, METALLOPROTEASES, MGDF receptor, MGMT, MHC(HLA-DR), MIF, MIG, MIP, MIP-1-alpha, MK, MMAC1, MMP, MMP-1, MMP-10, MMP-11, MMP-12, MMP-13, MMP-14, MMP-15, MMP-2, MMP-24, MMP-3, MMP-7, MMP-8, MMP-9, MPIF, Mpo, MSK, MSP, mucin (Muc1), MUC18, Muellerian-inhibitin substance, Mug, MuSK, NAIP, NAP, NAV 1.7, NCAD, N-Cadherin, NCA 90, NCAM, NCAM, Neprilysin, Neurotrophin-3, −4, or −6, Neurturin, Neuronal growth factor (NGF), NGFR, NGF-beta, nNOS, NO, NOS, Npn, NRG-3, NT, NTN, OB, OGG1, oncostatin M receptor (OSMR), OPG, OPN, OSM, OX40L, OX40R, p150, p95, PADPr, Parathyroid hormone, PARC, PARP, PBR, PBSF, PCAD, P-Cadherin, PCNA, PD1, PDL1, PDGF, PDGF, PDK-1, PECAM, PEM, PF4, PGE, PGF, PGI2, PGJ2, PIN, PLA2, placental alkaline phosphatase (PLAP), P1GF, PLP, PP14, Proinsulin, Prorelaxin, Protein C, PS, PSA, PSCA, prostate specific membrane antigen (PSMA), PTEN, PTHrp, Ptk, PTN, R51, RANK, RANKL, RANTES, RANTES, Relaxin A-chain, Relaxin B-chain, renin, respiratory syncytial virus (RSV) F, RSV Fgp, Ret, Rheumatoid factors, RLIP76, RPA2, RSK, S100, SCF/KL, SDF-1, SERINE, Serum albumin, sFRP-3, Shh, SIGIRR, SK-1, SLAM, SLPI, SMAC, SMDF, SMOH, SOD, SPARC, Stat, STEAP, STEAP-II, TACE, TACI, TAG-72 (tumor-associated glycoprotein-72), TARC, TCA-3, T-cell receptors (e.g., T-cell receptor alpha/beta), TdT, TECK, TEM1, TEM5, TEM7, TEM8, TERT, testicular PLAP-like alkaline phosphatase, TfR, TGF, TGF-alpha, TGF-beta, TGF-beta Pan Specific, TGF-beta R1 (ALK-5), TGF-beta R11, TGF-beta RIIb, TGF-beta RIII, TGF-betai, TGF-beta2, TGF-beta3, TGF-beta4, TGF-beta5, Thrombin, Thymus Ck-1, Thyroid stimulating hormone, Tie, TIMP, TIQ, Tissue Factor, TMEFF2, Tmpo, TMPRSS2, TNF, TNF-alpha, TNF-alpha beta, TNF-beta2, TNFc, TNF-RI, TNF-RII, TNFRSF10A (TRAIL R1Apo-2, DR4), TNFRSF10B (TRAIL R2DR5, KILLER, TRICK-2A, TRICK-B), TNFRSF10C (TRAIL R3DcR1, LIT, TRID), TNFRSF10D (TRAIL R4 DcR2, TRUNDD), TNFRSF1 IA (RANK ODF R, TRANCE R), TNFRSF11B (OPG OCIF, TR1), TNFRSF12 (TWEAK R FN14), TNFRSF13B (TACI), TNFRSF13C (BAFF R), TNFRSF14 (HVEM ATAR, HveA, LIGHT R, TR2), TNFRSF16 (NGFR p75NTR), TNFRSF17 (BCMA), TNFRSF18 (GITR AITR), TNFRSF19 (TROY TAJ, TRADE), TNFRSF19L (RELT), TNFRSF1A (TNF R1CD120a, p55-60), TNFRSF1B (TNF RII CD120b, p75-80), TNFRSF26 (TNFRH3), TNFRSF3 (LTbR TNF RIII, TNFC R), TNFRSF4 (OX40 ACT35, TXGP1R), TNFRSF5 (CD40 p50), TNFRSF6 (Fas Apo-1, APT1, CD95), TNFRSF6B (DcR3M68, TR6), TNFRSF7 (CD27), TNFRSF8 (CD30), TNFRSF9 (4-1BB CD137, ILA), TNFRSF21 (DR6), TNFRSF22 (DCTRAIL R2 TNFRH2), TNFRST23 (DCTRAIL R1TNFRH1), TNFRSF25 (DR3Apo-3, LARD, TR-3, TRAMP, WSL-1), TNFSF10 (TRAIL Apo-2 Ligand, TL2), TNFSFI 1 (TRANCE/RANK Ligand ODF, OPG Ligand), TNFSF12 (TWEAK Apo-3 Ligand, DR3Ligand), TNFSF13 (APRIL TALL2), TNFSF13B (BAFF BLYS, TALL1, THANK, TNFSF20), TNFSF14 (LIGHT HVEM Ligand, LTg), TNFSF15 (TL1A/VEGI), TNFSF18 (GITR Ligand AITR Ligand, TL6), TNFSF1A (TNF-α Conectin, DIF, TNFSF2), TNFSF1B (TNF-b LTa, TNFSF1), TNFSF3 (LTb TNFC, p33), TNFSF4 (OX40 Ligand gp34, TXGP1), TNFSF5 (CD40 Ligand CD154, gp39, HIGM1, IMD3, TRAP), TNFSF6 (Fas Ligand Apo-1 Ligand, APT1 Ligand), TNFSF7 (CD27 Ligand CD70), TNFSF8 (CD30 Ligand CD153), TNFSF9 (4-1BB Ligand CD137 Ligand), TP-1, t-PA, Tpo, TRAIL, TRAIL R, TRAIL-R1, TRAIL-R2, TRANCE, transferring receptor, TRF, Trk (e.g., TrkA), TROP-2, TSG, TSLP, tumor-associated antigen CA 125, tumor-associated antigen expressing Lewis Y related carbohydrate, TWEAK, TXB2, Ung, UPAR, uPAR-1, Urokinase, VCAM, VCAM-1, VECAD, VE-Cadherin, VE-cadherin-2, VEFGR-1 (fit-1), VEGF, VEGFR, VEGFR-3 (flt-4), VEGI, VIM, Viral antigens, VLA, VLA-1, VLA-4, VNR integrin, von Willebrands factor, WIF-1, WNT1, WNT2, WNT2B/13, WNT3, WNT3A, WNT4, WNT5A, WNT5B, WNT6, WNT7A, WNT7B, WNT8A, WNT8B, WNT9A, WNT9A, WNT9B, WNT10A, WNT10B, WNT11, WNT16, XCL1, XCL2, XCR1, XCR1, XEDAR, XIAP, XPD, and receptors for hormones and growth factor. In some embodiments, the antigen molecule is a SARS-CoV-2 protein. In some embodiments, the antigen molecule is a SARS-CoV-2 Spike protein.

### EXAMPLES

### Example 1. Canine Antibody Repertoire from Next-Generation Sequencing (NGS) of Canine Peripheral Blood Mononuclear Cells (PBMCs)

A canine phage display library was generated with canine germline frameworks that are abundantly expressed in the antibody repertoire found in dogs and expressed on bacteriophage. In addition, the frameworks for the library, when converted into canine IgGB, are expected to express at a relatively high level with mammalian cells and good thermostability. The CDRs (HCDR1, HCDR2, HCDR3, LCDR1, LCDR2, and LCDR3) for the library contain sequences natively found in dogs. As a first step for the library generation, the immune repertoire of dogs was characterized by NGS of PBMCs from different dogs (Table 8).

TABLE 8
Dogs used for NGS characterization of the immune repertoire

| Dog Number | Breed |
|---|---|
| 1 | Belgian Malinois |
| 2 | Belgian Sheepdog |
| 3 | Mixed |
| 4 | Plott Hound |
| 5 | Rottweiler |
| 6 | Siberian Husky |
| 7 | Pitbull |
| 8 | Great Pyrenese |
| 9 | American Staffordshire |
| 10 | German Shepherd |
| 11 | Hound Mix |
| 12 | German Shepherd |
| 13 | English Springer Spaniel |
| 14 | Boxer |
| 15 | Italian Spinone |
| 16 | Shetland Sheepdog |
| 17 | Border Collie |
| 18 | Poodle |
| 19 | Cocker Spaniel |
| 20 | Mixed |

PBMCs were isolated from dog blood using BD VACUTAINER® CPT™ tubes (BD Biosciences, 362753) using the recommended protocol from the manufacturer. Following the suspension of the cell pellet in 50 μL of phosphate buffered saline, pH 7.2, 250 μL of RNALATER® (ThermoFisher Scientific, AM7020) was added and frozen at −80° C. RNA was extracted using RNAQUEOUS™ Total RNA Isolation Kit (ThermoFisher, AM1912) as indicated by the manufacturer. cDNA was generated using the SUPERSCRIPT™ First-Strand Synthesis System for RT-PCR kit (ThermoFisher, 11904018) for each individual animal using an Oligo(dT) primer for first-strand synthesis. Amplification of canine VH, VK, and VL genes from cDNA was performed separately for each individual animal by PCR using pooled, gene-specific forward and reverse primers for their respective genes (Table 9). Amplified DNA was then isolated and purified by agarose gel electrophoresis.

TABLE 9
Primers used for NGS characterization of
canine antibody repertoire

| | Sequence | Sequence | Tₘ |
|---|---|---|---|
| Gene | Name | (5′ → 3′) | (° C.) |
| Heavy | Canine | GAGGAGCAACTGGTGGA | 68.2 |
| Chain | IGHV | GTTTGGAGGACACATGG | |
| Forward | FOR 1 | TGAATCC | |
| | | (SEQ ID NO: 332) | |
| | Canine | GAACTCACACTGCAGGA | 69.1 |
| | IGHV | GTCAGGGCCAGGACTGG | |
| | FOR 2 | (SEQ ID NO: 333) | |
| | Canine | GAGGTGCAGCTGGTGGA | 68.3 |
| | IGHV | GTCTGGGGG | |
| | FOR 3 | (SEQ ID NO: 334) | |
| | Canine | GAGGTACAGCTGGTGGA | 65.7 |
| | IGHV | GTCTGGGGG | |
| | FOR 4 | (SEQ ID NO: 335) | |
| | Canine | GAGGTACAGCTGGTGGA | 64.3 |
| | IGHV | ATCTGGGGG | |
| | FOR 5 | (SEQ ID NO: 336) | |
| | Canine | GAGGTGCAGCTGGTGGA | 66.2 |
| | IGHV | GTCTGGAGG | |
| | FOR 6 | (SEQ ID NO: 337) | |
| | Canine | GAGGTCCAGCTGGTGCA | 68.6 |
| | IGHV | GTCTGGGGC | |
| | FOR 7 | (SEQ ID NO: 338) | |
| Kappa | Canine | GATATTGTCATGACACA | 66.6 |
| Light | IGKV | GACCCCACTGTCCCTGT | |
| Chain | FOR 1 | CCG | |
| Forward | | (SEQ ID NO: 339) | |
| | Canine | GATATCGTCATGACACA | 67.5 |
| | IGKV | GACCCCACTGTCCCTGT | |
| | FOR 2 | CCG | |
| | | (SEQ ID NO: 340) | |
| | Canine | GAGGCCGTGATGACGCA | 68.5 |
| | IGKV | GACCCCACTGTC | |
| | FOR 3 | (SEQ ID NO: 341) | |
| | Canine | GAAATCGTGATGACACA | 66.7 |
| | IGKV | GTCTCCAGCCTCCCTCT | |
| | FOR 4 | CC | |
| | | (SEQ ID NO: 342) | |
| Lambda | Canine | TTGCCCGTGCTGACCCA | 69.4 |
| Light | IGLV | GCCTCCAAGTG | |
| Chain | FOR 1 | (SEQ ID NO: 343) | |
| Forward | Canine | CAGGCTGTGCTGACTCA | 68.7 |
| | IGLV | GCCACCCTCTGTGTC | |
| | FOR 2 | (SEQ ID NO: 344) | |
| | Canine | CAGTCTGCCCTGACTCA | 68.4 |
| | IGLV | ACCTTCCTCGGTGTCTG | |
| | FOR 3 | G | |
| | | (SEQ ID NO: 345) | |
| | Canine | CAGTCTATGCTGACTCA | 67.1 |
| | IGLV | GCCAGCCTCAGTGTCTG | |
| | FOR 4 | G | |
| | | (SEQ ID NO: 346) | |
| | Canine | CAGTCTGTGCTGACTCA | 68.6 |
| | IGLV | GCCGGCCTCAGTGTC | |
| | FOR 5 | (SEQ ID NO: 347) | |
| | Canine | CAGTCTGTGCTGACTCA | 68.7 |
| | IGLV | ACCAGCCTCAGTGTCCG | |
| | FOR 6 | G | |
| | | (SEQ ID NO: 348) | |
| | Canine | CAGTCTGTGCTGACTCA | 68.8 |
| | IGLV | GCCGACCTCAGTGTCGG | |
| | FOR 7 | (SEQ ID NO: 349) | |
| | Canine | TCCTATGTGCTGTCTCA | 68.8 |
| | IGLV | GCCGCCATCAGCGACTG | |
| | FOR 8 | (SEQ ID NO: 350) | |
| | CanineI | TCCTATGTGCTGACACA | 67.7 |
| | GLV | GCTGCCATCCATGAGTG | |
| | FOR 9 | TGAC | |
| | | (SEQ ID NO: 351) | |
| Heavy | Canine_ | TGAGGAGACGGTGACCA | 68 |
| Chain | IGHJ | GGGTGCCC | |
| Reverse | REV 1 | (SEQ ID NO: 352) | |
| | Canine_ | TGAGGAGACAGTGACCA | 68.2 |
| | IGHJ | GGGTGCCCTGG | |
| | REV 2 | (SEQ ID NO: 353) | |
| | Canine_ | TGAGGAGACGGTGACCA | 67.9 |
| | IGHJ | GGGTTCCCTGG | |
| | REV 3 | (SEQ ID NO: 354) | |
| | Canine_ | TGAGGACACAGTGACCA | 68 |
| | IGHJ | GGGTCCCTTGGC | |
| | REV 4 | (SEQ ID NO: 355) | |
| | Canine_ | TGAGGACACGAAGAGTG | 67.7 |
| | IGHJ | AGGTGCCATGGCC | |
| | REV 5 | (SEQ ID NO: 356) | |
| Kappa | Canine_ | CGGAGCAGGAACCAAGG | 65.6 |
| Light | IGJK | TGGAGCTCAAA | |
| Chain | REV 1 | (SEQ ID NO: 357) | |
| Reverse | Canine_ | TTTGATCTCCAGTTTGG | 62.7 |
| | IGJK | TCCCTTGGCC | |
| | REV 2 | (SEQ ID NO: 358) | |
| | Canine_ | TTTGATCTCCACCTTGG | 65.6 |
| | IGJK | TCCCTTGGCCG | |
| | REV 3 | (SEQ ID NO: 359) | |
| | Canine_ | GGCAAAGGGACACATCT | 58.9 |
| | IGJK | GGAGATTAAA | |
| | REV 4 | (SEQ ID NO: 360) | |
| Lambda | Canine_ | GAGGACGGTCAGCTGGG | 68.6 |
| Light | IGJL | TCCCTTCACCG | |
| Chain | REV 1 | (SEQ ID NO: 361) | |
| Reverse | Canine_ | GAGGATGGTCAGCTGGG | 68.7 |
| | IGJL | TCCCTCTGCCG | |
| | REV 2 | (SEQ ID NO: 362) | |
| | Canine_ | AAGGACGGTCAGTTGGG | 67 |
| | IGJL | TTCCTGAGCCG | |
| | REV 3 | (SEQ ID NO: 363) | |
| | Canine | GAGGACGGTCAGATGGG | 68.2 |
| | IGJL | TGCCTCCGC | |
| | REV 4 | (SEQ ID NO: 364) | |
| | Canine_ | GAGGACGGTCAGGTGGG | 68.5 |
| | IGJL | TGCCTCCG | |
| | REV 5 | (SEQ ID NO: 365) | |

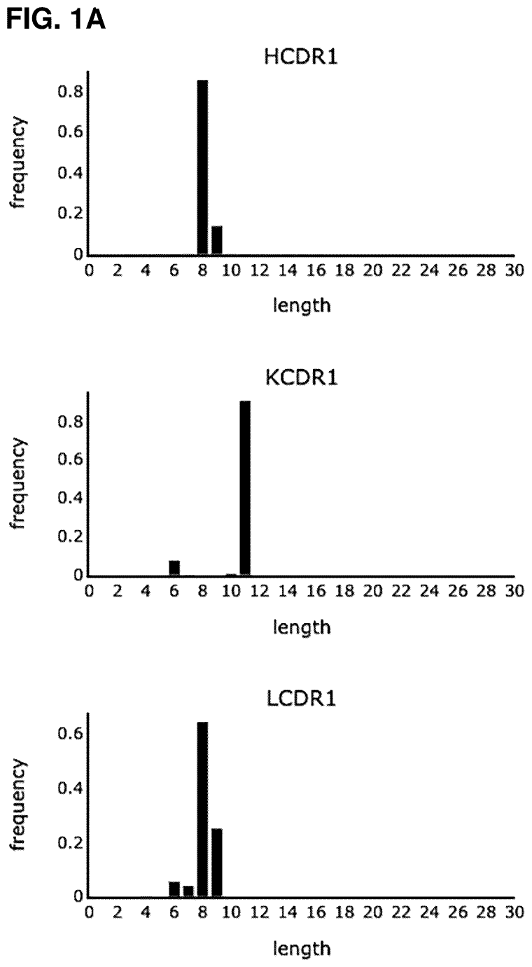

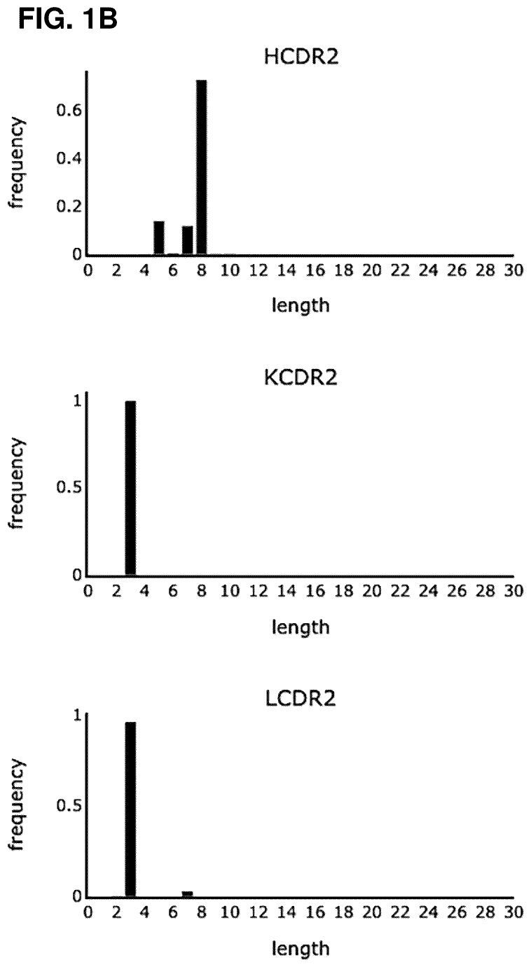

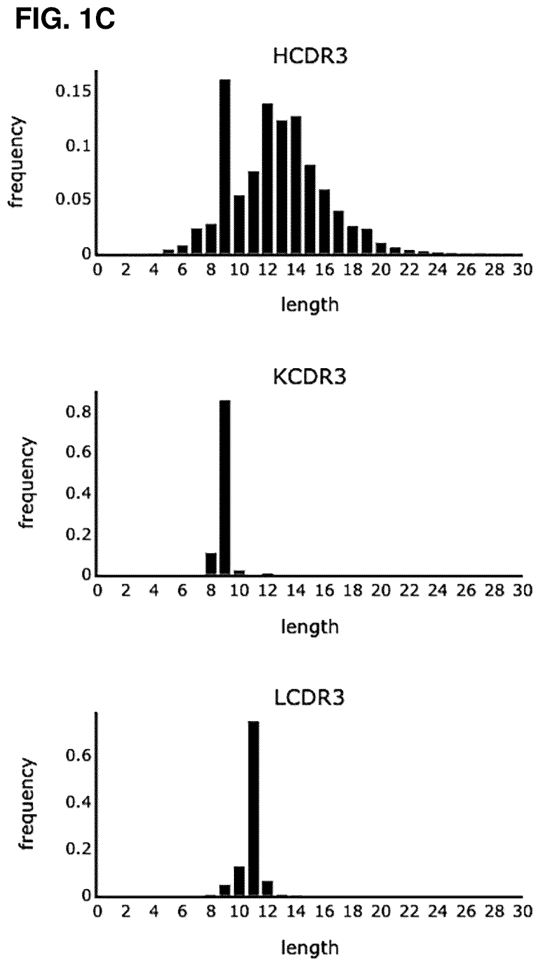

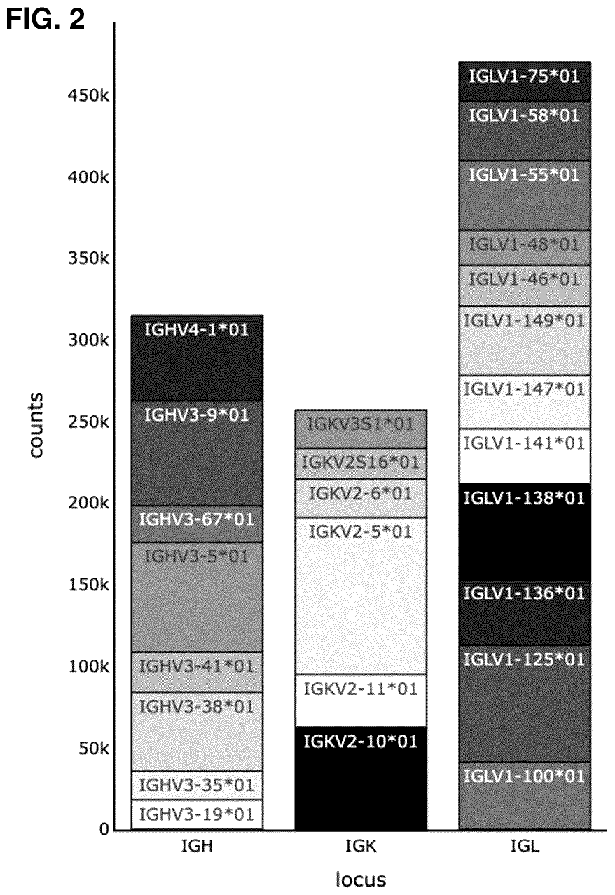

NGS sample preparation was performed using Twist Bioscience Library Preparation Kit with Amp Mix (Twist Biosciences) as indicated by the manufacturer. In brief, end-repair and Poly(A)-tailing was performed followed by ligation of universal adapters. PCR was then used to incorporate unique dual-index (UDI) barcodes. Each animal's amplified VH, VK, and VL gene pools were individually barcoded so that the diversity for each sample could be traced back to the specific donor. Pooled NGS sample library was then run on an Illumina MISEQ™ using the MISEQ™ Reagent Kits v3 (600 cycle) kit (Illumina, MS-102-3003). Sequencing files were first demultiplexed, followed by trimming and joining paired reads (Q25=99.6% accuracy for base calling, min length filter ˜600). Annotation and segmentation of VDJ sequences was then performed using a custom developed software for germline assignment. The number of unique CDRs identified are shown in Table 10. The distribution of CDR lengths is shown in FIGS. 1A-1C. Also, the frequency of the different frameworks is shown in FIG. 2.

TABLE 10
Unique CDR sequences identified in canine antibody repertoire

| CDR | Chain | Number of Unique CDRs |
|---|---|---|
| CDR1 | IGH | 20,370 |
| | IGK | 8,573 |
| | IGL | 33,175 |
| CDR2 | IGH | 40,547 |
| | IGK | 480 |
| | IGL | 3,471 |
| CDR3 | IGH | 106,881 |
| | IGK | 10,585 |
| | IGL | 82,450 |

### Example 2. Combinations of Canine VH and VK/VL Germlines Expressed on Phage

Display of paired canine VH and VK or VL as a single-chain variable fragment (scFv) was performed as follows. The top 20 most abundant VH were paired with either the top 5 most abundant VK or 15 most abundant VL genes (Table 11) observed in the canine antibody repertoire NGS sequencing. Each pairing of VH and VL frameworks was tested. Synthetic double-stranded DNA fragments (Twist Biosciences) encoding each individual VH and VL pairing were synthesized, with a (G4S)₃linker (GGGGSGGGGSGGGGS, SEQ ID NO: 331) between the VH and VL sequences as well as flanking sequences homologous to the recipient phagemid vector, pADL22c (Antibody Design Laboratories). Linear pADL22c was obtained through restriction digest with Sfil, followed by purification by agarose gel electrophoresis. Subcloning was performed using NEBUILDER® HiFi DNA Assembly Master Mix (New England Biolabs, E2621X) as instructed by the manufacturer. DNA was then transformed into electrocompetent TG1 E. coli(Lucigen, 50-104-7949), and clones were verified by Sanger sequencing.

TABLE 11
Canine VH, VK, and VL germlines used in library construction

| IGHV Germline | IGKV Germline | IGLV Germline |
|---|---|---|
| IGHV3-38*01 | IGKV2-5*02 | IGLV1-138*01 |
| IGHV3-19*01 | IGKV2-11*01 | IGLV1-55*01 |
| IGHV3-35*01 | IGKV2-10*01 | IGLV1-136*01 |
| IGHV3-5*01 | IGKV2-16*01 | IGLV1-141*01 |
| IGHV4-1*01 | IGKV3-18*01 | IGLV3-11*01 |
| IGHV3-67*01 | | IGLV1-75*01 |
| IGHV3-69*01 | | IGLV1-125*01 |
| IGHV3-9*01 | | IGLV1-48*01 |
| IGHV3-50*01 | | IGLV1-46*01 |
| IGHV3-24*01 | | IGLV3-14*01 |
| IGHV3-47*01 | | IGLV1-100*01 |
| IGHV3-41*01 | | IGLV1-103*01 |
| IGHV3-2*01 | | IGLV1-149*01 |
| IGHV3-39*01 | | IGLV4-22*01 |
| IGHV1-30*01 | | IGLV2-31*01 |
| IGHV3-6*01 | | |
| IGHV3-81*01 | | |
| IGHV3-54*01 | | |
| IGHV3-7*01 | | |
| IGHV3-3*01 | | |

Determining the level of scFv display on M13 phage was performed for each of the VH and VK/VL pairs (Tables 12 and 13). Each variable heavy and variable clones were grown in 2xYT media supplemented with 100 μg/mL carbenicillin and 2% (w/v) glucose (2xYT-CG) overnight with shaking at 30° C. Overnight cultures were inoculated into 1 mL of fresh pre-warmed 2xYT-CG media and grown to an OD₆₀₀=0.5 at 37° C. followed by infection with 2.2×108 pfu of M13K07 helper phage for 30 minutes of static incubation and 30 minutes of shaking incubation at 37° C. Cultures were then pelleted by centrifugation at 4,000×g for 10 minutes and resuspended in 2xTY supplemented with 100 μg/mL carbenicillin and 50 μg/mL kanamycin and grown overnight at 30° C. with shaking. The following day, cultures were pelleted by centrifugation, and clarified supernatant was diluted in phosphate-buffered saline (PBS) supplemented with 0.05% TWEEN®-20, 3% (w/v) powdered non-fat milk, and 0.5% bovine serum albumin (BSA) (PBST-MB).

TABLE 12
scFv display level of canine VH and VK pairs

| | IGKV2-5 | IGKV2-11 | IGKV2-10 | IGKV2-16 | IGKV3-18 |
|---|---|---|---|---|---|
| IGHV3-38 | 3.51 | 2.33 | 1.36 | 0.9 | 1.51 |
| IGHV3-19 | 1.33 | 1.47 | 1.09 | 1.14 | 1.08 |
| IGHV3-35 | 11.36 | 1.14 | 0.96 | 2.61 | 2.44 |
| IGHV3-5 | 3.83 | 2.87 | 1.1 | 6.95 | 1.78 |
| IGHV4-1 | 2.06 | 3.15 | 1.41 | 1.07 | 0.85 |
| IGHV3-67 | 1.17 | 1.24 | 1.19 | 2.1 | 1.19 |
| IGHV3-69 | 0.93 | 8.24 | 12.4 | 1.16 | 1.22 |
| IGHV3-9 | 1.3 | 4.06 | 1.32 | 12.25 | 12.9 |
| IGHV3-50 | 2.92 | 1.93 | 1.49 | 1.34 | 2.13 |
| IGHV3-24 | 1.16 | 1.3 | 2.28 | 1.29 | 1.17 |
| IGHV3-47 | 0.81 | 2.13 | 1.44 | 0.95 | 2.52 |
| IGHV3-41 | 0.88 | 1.75 | 1.79 | 1.15 | 4.94 |
| IGHV3-2 | 3.22 | 3.11 | 2.8 | 2.4 | 1.55 |
| IGHV3-39 | 1.01 | 1.3 | 1.21 | 1.37 | 0.86 |
| IGHV1-30 | 0.89 | 1.12 | 1.23 | 1.13 | 1.27 |
| IGHV3-6 | 1.47 | 1.6 | 1.18 | 1.02 | 1.32 |
| IGHV3-81 | 16.43 | 14.09 | 15.15 | 5.02 | 7.05 |
| IGHV3-54 | 2.12 | 16.62 | 1.25 | 25.49 | 2.82 |
| IGHV3-7 | 14.59 | 3.28 | 11.44 | 16.01 | 4.22 |
| IGHV3-3 | 6.75 | 4.61 | 4.88 | 19.01 | 4.53 |

TABLE 13
scFv display level of canine VH and VL pairs

| | IGLV1-138 | IGLV1-55 | IGLV1-136 | IGLV1-141 | IGLV3-11 | IGLV1-75 | IGLV1-125 | IGLV1-48 |
|---|---|---|---|---|---|---|---|---|
| IGHV3-38 | 2.06 | 2.96 | 13.31 | 2.86 | 2 | 9.16 | 3.67 | 2.38 |
| IGHV3-19 | 1.11 | 2.64 | 0.98 | 1.46 | 2.04 | 12.29 | 1.22 | 15.86 |
| IGHV3-35 | 5.9 | 3.01 | 1.11 | 10.43 | 0.9 | 1.06 | 18.48 | 7.15 |
| IGHV3-5 | 3.31 | 2.72 | 3.59 | 9.93 | 1.89 | 1.48 | 1.01 | 5.54 |
| IGHV4-1 | 1.7 | 9.79 | 2.28 | 1.83 | 2 | 1.88 | 1.1 | 3.39 |
| IGHV3-67 | 2.13 | 1.05 | 1.56 | 6.3 | 1.07 | 1.22 | 3.26 | 7.05 |
| IGHV3-69 | 4.94 | 7.18 | 1.59 | 1.67 | 2.71 | 2.89 | 6.46 | 5.57 |
| IGHV3-9 | 21.36 | 2.96 | 2.91 | 6.6 | 5.73 | 1.64 | 9.45 | 17.25 |
| IGHV3-50 | 2.78 | 3.59 | 1.26 | 1.29 | 1.63 | 4.43 | 2.19 | 19.26 |
| IGHV3-24 | 1.03 | 1.13 | 3.98 | 1.71 | 1.64 | 1.34 | 2.1 | 4.11 |
| IGHV3-47 | 1.61 | 2.26 | 1.74 | 10.56 | 1.61 | 2.57 | 15.39 | 8.42 |
| IGHV3-41 | 1.31 | 15.57 | 3.53 | 3.57 | 2.36 | 2.32 | 22.77 | 14.5 |
| IGHV3-2 | 2.08 | 1.87 | 5.13 | 2.21 | 1.41 | 2.08 | 6.67 | 11.42 |
| IGHV3-39 | 1.3 | 2.47 | 3.01 | 2.32 | 2.44 | 1.18 | 2.61 | 7.38 |
| IGHV1-30 | 1.16 | 1.34 | 1.59 | 1.45 | 1.69 | 1.18 | 1.33 | 5.74 |
| IGHV3-6 | 1.3 | 1.59 | 1.8 | 1.7 | 1.54 | 1.32 | 1.72 | 4.37 |
| IGHV3-81 | 30.36 | 25.83 | 9.23 | 13.21 | 7.24 | 15.7 | 14.91 | 19.42 |
| IGHV3-54 | 7.97 | 3.25 | 7.58 | 16.02 | 3.93 | 6.73 | 6.09 | 3.57 |
| IGHV3-7 | 7.17 | 4.57 | 3.96 | 4.27 | 6.33 | 6.21 | 13.07 | 8.69 |
| IGHV3-3 | 15.41 | 4.58 | 4.05 | 3.23 | 6.3 | 3.62 | 9.41 | 7.33 |

| | IGLV1-46 | IGLV3-14 | IGLV1-100 | IGLV1-103 | IGLV1-149 | IGLV4-22 | IGLV2-31 |
|---|---|---|---|---|---|---|---|
| IGHV3-38 | 2.52 | 2.72 | 3.23 | 5.62 | 7.01 | 2.36 | 2.3 |
| IGHV3-19 | 7.26 | 8.81 | 8.55 | 8.17 | 8.02 | 2.92 | 2.98 |
| IGHV3-35 | 2.16 | 2.37 | 2 | 6.97 | 4.34 | 5.8 | 0.84 |
| IGHV3-5 | 2.56 | 4.59 | 6.02 | 5.97 | 4.98 | 6.34 | 6.58 |
| IGHV4-1 | 3.54 | 5.06 | 4.91 | 3.67 | 2.03 | 4.08 | 3.79 |
| IGHV3-67 | 2.99 | 3.15 | 9.99 | 2.94 | 6.17 | 5.11 | 3.66 |
| IGHV3-69 | 9.02 | 5.07 | 9.75 | 2.78 | 7.54 | 4.88 | 7.09 |
| IGHV3-9 | 4.83 | 17.97 | 17.32 | 17.02 | 10.16 | 4.67 | 8.39 |
| IGHV3-50 | 14.98 | 9.12 | 14.07 | 14.05 | 11.22 | 6.91 | 15.38 |
| IGHV3-24 | 4.84 | 2.14 | 2.82 | 3.78 | 1.79 | 2.76 | 2.23 |
| IGHV3-47 | 6.72 | 4.54 | 5.79 | 7.01 | 2.57 | 1.42 | 4.03 |
| IGHV3-41 | 13.46 | 13.2 | 12.09 | 11.67 | 11.67 | 9.56 | 11.17 |
| IGHV3-2 | 13.13 | 12.41 | 16.27 | 13.72 | 14.36 | 8.74 | 13.09 |
| IGHV3-39 | 5.3 | 6.34 | 7.05 | 7.05 | 4.61 | 3.24 | 4.55 |
| IGHV1-30 | 2.6 | 2.38 | 3.63 | 3.99 | 2.5 | 2.03 | 1.56 |
| IGHV3-6 | 6.81 | 3.85 | 5.83 | 6.01 | 2.16 | 3.63 | 3.52 |
| IGHV3-81 | 9.19 | 9.54 | 19.41 | 6.52 | 7.12 | 2.37 | 3.71 |
| IGHV3-54 | 8 | 11.53 | 4.24 | 3.05 | 9.04 | 1.86 | 4.6 |
| IGHV3-7 | 9.38 | 4.33 | 6.65 | 6.29 | 12.53 | 3.78 | 3.92 |
| IGHV3-3 | 6.41 | 5.03 | 4.31 | 6.3 | 8.69 | 3.27 | 3.5 |

NUNC™ MAXISORP™ 96 well plates were coated with 0.2 μg mouse anti-histidine tag antibody (R&D, MAB050-100) or BSA for background measurements in 50 mM sodium carbonate, pH 9.5, overnight at 4° C. Plates were washed 3 times with PBS supplemented with TWEEN®-20 (PBS-T) and blocked with PBST-MB at 25° C. for 2 hours with shaking. Plates were then washed 3 times with PBS-T, and phage supernatant diluted in PBST-MB was applied to the well and allowed to incubate at 25° C. for 2 hours with shaking. Plates were then washed 3 times with PBS-T, and 1:5000 mouse anti-M13 g8p antibody HRP (Antibody Design Laboratories, AS003) diluted in PBST-MB was applied to the well for 1 hour with shaking. Plates were then washed 6 times with PBS-T and TMB substrate (3,3′,5,5′ tetramethylbenzidine) was added to develop the ELISA. Stop solution (1 M phosphoric acid) was added to the well, and absorbance at 450 nm was recorded on a Tecan SPARK® plate reader. Signal/background ratio (S/B) was calculated by the following equation (Equation 1) and normalized to phage concentration.

$$\frac{S}{B} = \frac{{OD}_{450 , His mAb}}{{OD}_{450 , BSA}} \qquad Equation 1$$

### Example 3. Expression, Purification and Thermal Stability of Canine Antibodies

Expression and purification of canine IgG antibodies was performed as follows. Double-stranded DNA fragments encoding the various heavy chain frameworks were fused with the CH1-CH2-CH3 domain of canine IgGB and synthesized by Twist Bioscience. The amino acid sequence of the CH1-CH2-CH3 domain of canine IgGB is provided below:

CH1-CH2-CH3 domain of canine IgGB:
(SEQ ID NO: 366)
ASTTAPSVFPLAPSCGSTSGSTVALACLVSGYFPEPVTVSWNSGSLTSGV
HTFPSVLQSSGLYSLSSMVTVPSSRWPSETFTCNVAHPASKTKVDKPVPK
RENGRVPRPPDCPKCPAPEMLGGPSVFIFPPKPKDTLLIARTPEVTCVVV
DLDPEDPEVQISWFVDGKQMQTAKTQPREEQFNGTYRVVSVLPIGHQDWL
KGKQFTCKVNNKALPSPIERTISKARGQAHQPSVYVLPPSREELSKNTVS
LTCLIKDFFPPDIDVEWQSNGQQEPESKYRTTPPQLDEDGSYFLYSKLSV
DKSRWQRGDTFICAVMHEALHNHYTQESLSHSPGK

The frameworks for kappa and lambda light chains were fused to the constant domain of the kappa chain or the constant domain of the lambda chain, respectively, and synthesized by Twist Bioscience. The amino acid sequences of the constant domain of the canine kappa chain or the constant domain of the canine lambda chain are provided below:

Constant domain of the canine kappa chain:
(SEQ ID NO: 367)
RNDAQPAVYLFQPSPDQLHTGSASVVCLLNSFYPKDINVKWKVDGVIQDT
GIQESVTEQDKDSTYSLSSTLTMSSTEYLSHELYSCEITHKSLPSTLIKS
FQRSECQRVD
Constant domain of the canine lambda chain:
(SEQ ID NO: 368)
GQPKASPSVTLFPPSSEELGANKATLVCLISDFYPSGVTVAWKADGSPVT
QGVETTKPSKQSNNKYAASSYLSLTPDKWKSHSSFSCLVTHEGSTVEKKV
APAECS

Fragments were subcloned into Notl/Xbal restriction digested pTwist CMV BG WPRE Neo linear vector using NEBUILDER® HiFi DNA Assembly Master Mix (NEB, E2621). All vector DNAs were sequence verified and scaled up for transient transfection into EXPI293F™ cells (ThermoFisher, Al 4527).

Transient transfections were performed as instructed by the manufacturer. EXPI293F™ cells were cultured at 37° C. with 8% CO₂ in EXPI293F™ Expression Medium (ThermoFisher, Al 435101) and diluted to a density of 3×10⁶ cells/mL. An equal ratio of plasmid DNA encoding the heavy and light chain for each respective pairing was used for transfection. Vector DNA (1 μg/mL of culture) was diluted into OPTI-MEM™ I media, as was EXPIFECTAMINE™ 293 reagent (ThermoFisher, Al 4525). The solutions were combined and allowed to complex before adding to the cell suspension. Enhancers were added as described by the manufacturer and cultures were then harvested on day 5. Supernatant was collected by centrifugation at 4,000×g for 20 minutes at 4° C. and applied to a 0.22 μm filter.

Canine antibodies were purified using a Hamilton MICROLAB® STAR automated platform. Antibodies were captured with 20 μL Phynexus tips (Biotage, 91-10-01) loaded with MABSELECT PRISMA™ protein A chromatography resin (Cytiva, 17549801) and washed with PBS. Antibodies were eluted with 50 mM citrate (pH 3.0) and immediately neutralized with 1 M HEPES, pH 8.0. Protein concentration was determined using a LUNATIC™ UV/Vis spectrophotometer (Unchained Labs), and protein purity was determined on the LABCHIP® GXII microfluidic capillary electrophoresis (CE-SDS) platform. Melting temperature (Tm) and aggregation temperature (T_{agg}) were determined on the UNCLE™ instrument (Unchained Labs) using the manufacturer's protocol (Table 14).

TABLE 14
Thermostability and protein yield for selected
VH and VL pairings as canine IgGB antibodies

| | | Average | Average | Yield |
|---|---|---|---|---|
| IGHV | IGKV/IGLV | Tₘ1 (° C.) | T_{agg} 266 (° C.) | (ug) |
| IGHV3-38 | IGKV2-5 | 71.8 | 61.5 | 52.80 |
| IGHV3-38 | IGLV1-55 | 70.6 | 71.7 | 68.20 |
| IGHV3-38 | IGLV1-136 | 67.6 | 68.2 | 105.60 |
| IGHV3-38 | IGLV1-75 | 67.3 | 68.4 | 99.00 |
| IGHV3-38 | IGLV1-125 | 71.5 | 70.5 | 17.20 |
| IGHV3-38 | IGLV1-100 | 67.3 | 68.3 | 97.32 |
| IGHV3-38 | IGLV1-103 | 65.7 | 65.5 | 87.31 |
| IGHV3-38 | IGLV1-149 | 69.3 | 69.4 | 108.81 |
| IGHV3-19 | IGLV1-75 | 59.1 | 59.5 | 37.04 |
| IGHV3-19 | IGLV1-48 | 62.7 | 63.5 | 88.74 |
| IGHV3-19 | IGLV1-46 | 63.1 | 64.3 | 86.92 |
| IGHV3-19 | IGLV3-14 | 61.2 | 61.4 | 73.63 |
| IGHV3-19 | IGLV1-100 | 64.1 | 65.2 | 49.54 |
| IGHV3-19 | IGLV1-103 | 62.5 | 62.5 | 51.37 |
| IGHV3-19 | IGLV1-149 | 61.6 | 61.7 | 105.77 |
| IGHV3-35 | IGKV2-5 | 66.4 | 65.4 | 96.64 |
| IGHV3-35 | IGLV1-138 | 62.5 | 62.6 | 80.45 |
| IGHV3-35 | IGLV1-55 | 63.9 | 63.7 | 89.40 |
| IGHV3-35 | IGLV1-141 | 64.7 | 63.8 | 95.37 |
| IGHV3-35 | IGLV1-125 | 64.5 | 64.4 | 44.17 |
| IGHV3-35 | IGLV1-103 | 64.5 | 62.9 | 22.30 |
| IGHV3-35 | IGLV1-149 | 62 | 60.9 | 118.84 |
| IGHV3-5 | IGKV2-5 | 67.4 | 68.1 | 45.12 |
| IGHV3-5 | IGKV2-16 | 67.7 | 68.1 | 112.77 |
| IGHV3-5 | IGLV1-138 | 69.6 | 69.8 | 114.79 |
| IGHV3-5 | IGLV1-136 | 69.3 | 69.8 | 105.02 |
| IGHV3-5 | IGLV1-141 | 70.9 | 71.7 | 37.26 |
| IGHV3-5 | IGLV1-48 | 69.6 | 69.9 | 112.92 |
| IGHV3-5 | IGLV3-14 | 68 | 68.9 | 65.47 |
| IGHV3-5 | IGLV1-100 | 70 | 70.9 | 111.93 |
| IGHV3-5 | IGLV1-103 | 68.2 | 68.6 | 96.82 |
| IGHV3-5 | IGLV1-149 | 69.7 | 70 | 115.03 |
| IGHV4-1 | IGKV2-11 | 69.2 | 68.6 | 96.42 |
| IGHV4-1 | IGLV1-55 | 71.6 | 70.8 | 110.85 |
| IGHV4-1 | IGLV1-48 | 71.3 | 70.4 | 102.89 |
| IGHV4-1 | IGLV1-46 | 71.8 | 71.4 | 114.24 |
| IGHV4-1 | IGLV3-14 | 70.6 | 69.7 | 96.822 |
| IGHV4-1 | IGLV1-100 | 72.1 | 71.4 | 120.86 |
| IGHV4-1 | IGLV1-103 | 71.1 | 69.7 | 96.99 |
| IGHV3-67 | IGLV1-141 | 69 | 70.3 | 85.18 |
| IGHV3-67 | IGLV1-125 | 69.4 | 70.3 | 125.86 |
| IGHV3-67 | IGLV1-48 | 70.1 | 69.9 | 42.98 |
| IGHV3-67 | IGLV1-46 | 68.1 | 69 | 106.32 |
| IGHV3-69 | IGKV2-11 | 67.8 | 67.8 | 52.84 |
| IGHV3-69 | IGKV2-10 | 70 | 70 | 71.21 |
| IGHV3-9 | IGKV2-11 | 69.2 | 68.4 | 65.51 |
| IGHV3-9 | IGKV2-16 | 69.2 | 68.8 | 68.59 |
| IGHV3-9 | IGKV3-18 | 68.5 | 68.4 | 39.13 |
| IGHV3-2 | IGKV2-5 | 70.9 | 68.7 | 46.24 |
| IGHV3-2 | IGKV2-11 | 71.2 | 68 | 63.29 |

### Example 4. Synthetic Phage Library Design and Construction

Synthetic canine libraries were constructed as follows. Using the data collected from the NGS antibody repertoire sequencing along with the display, expression, and thermostability data, three pairings of VH and VL germline frameworks were selected for the synthetic library design. The three pairing libraries were IGFV3-5/IGLV1-100 (Library 1), IGHV3-5/IGLV1-103 (Library 2), and IGHV4-1/IGLV1-48 (Library 3). CDRs for both VH and VL were sourced from the respective NGS. Only CDRs linked with the chosen frameworks as identified with NGS were used for each library. CDRs containing potential sequence liabilities were removed from the pool of CDR content. These included sequences containing a glycosylation motif (NXS/T, where X is any amino acid except proline), fragmentation motifs, unpaired cysteines, and methionine in HCDR3. Three distinct libraries with the selected IGHV/IGLV pairings were synthetically produced and assembled into scFv linear fragments using Twist Biosciences proprietary technology (Table 15).

TABLE 15
Summary of synthetic canine phage display libraries

| | | | | | | | | | | Full |
|---|---|---|---|---|---|---|---|---|---|---|
| | IGHV-IGLV | Number of sequences for each CDR | | | | | | Theoretical | Transformed | length |
| Library | Pairing | HCDR1 | HCDR2 | HCDR3 | LCDR1 | LCDR2 | LCDR3 | Diversity | Diversity | ORF |
| 1 | IGFHV3-5, | 1473 | 1322 | 8868 | 2019 | 1054 | 2069 | 7.60E+19 | 5.00E+09 | 85% |
| | IGLV1-100 | | | | | | | | | |
| 2 | IGHV3-5, | 1473 | 1322 | 8868 | 893 | 708 | 1040 | 1.14E+19 | 1.80E+10 | 75% |
| | IGLV1-103 | | | | | | | | | |
| 3 | IGHV4-1, | 2533 | 2768 | 6539 | 1037 | 842 | 1339 | 5.36E+19 | 4.00E+10 | 90% |
| | IGLV1-48 | | | | | | | | | |

The final scFv libraries all contain a 15 amino acid (G4S)₃ linker between the heavy and light chain. Linear library fragments were purified by agarose gel electrophoresis. pADL22c vector was linearized by Sfil restriction digest and purified by gel electrophoresis prior to cloning the library. The library was assembled by Twist Biosciences proprietary assembly reaction, and DNA was further purified by ethanol precipitation prior to electroporation into electrocompetent TG1 E. coli. A total of 15 electroporation reactions were performed using a GENE PULSER XCELL™ Electroporation System (Bio-Rad). Cells were immediately resuspended with pre-warmed recovery media (Lucigen, 80026-1) and allowed to incubate for 1 hour at 37° C. with shaking. Following culture recovery, 10 μL of cells were collected for determining the transformed library size (Equation 2, Table 15). The remaining cells were pelleted by centrifugation at 6500×g for 10 minutes at 4° C., resuspended in 1 mL of 2xYT media, and plated on 2xYT agar plates supplemented with 100 μg/mL carbenicillin and 2% (w/v) glucose. Plates were allowed to grow overnight at 30° C. E. coli lawns were harvested and stored as glycerol stock at −80° C. The percentage of scFv containing open frames was determined from Sanger sequencing of 96 individual clones (Table 15).

$$Equation 2$$

$$Transformed Library size ( CFU ) = \frac{\begin{matrix}Vol u m e of rescue culture \times \\ Number of colonies \times Dilution factor\end{matrix}}{Volume of culture plated out}$$

Phage libraries were produced as follows. A glycerol stock of each library was used to inoculate 1 L of pre-warmed 2xYT-GC media to an OD₆₀₀=0.15. Cultures were allowed to grow with shaking to an OD₆₀₀=0.5 followed by infection with 2.2×10¹¹ pfu of M13K07 helper phage for 30 minutes of static incubation and 30 minutes of shaking incubation at 37° C. Cultures were then pelleted by centrifugation at 6,500×g for 10 minutes, resuspended in 2xTY supplemented with 100 μg/mL carbenicillin and 50 μg/mL kanamycin, and grown overnight at 30° C. with shaking. Clarified supernatant was collected by centrifugation at 6,500×g for 10 minutes and % volume of PEG/NaCl was added to the supernatant and allowed to incubate on ice for 1 hour. Phage was then pelleted by centrifugation at 10,000×g for 30 minutes at 4° C. The supernatant was discarded, and the pellet containing phage was resuspended in PBS. This solution was then centrifuged at 6,000×g for 10 minutes at 4° C. to remove any remaining cellular debris. A second PEG/NaCl precipitation was performed following the steps described above. Pelleted phage was resuspended in PBS supplemented with 0.5% (w/v) BSA and 20% (v/v) glycerol at an OD₂₆₈=1.0 and stored at −80° C.

### Example 5. Canine Phage Library Selections and Screening with SARS-CoV-2 S1 Protein

The three libraries were evaluated for their performance by performing selections and screens with recombinant SARS-CoV-2 S1 protein (Acro Biosystems, S1 N-C52H3). Biotinylated SARS-CoV-2 S1 protein was diluted into PBS-T (PBS+0.1% TWEEN®-20) supplemented with 0.5% BSA (w/v) (PBST-B). DYNABEADS™ M-280 Streptavidin (ThermoFisher, 11205D) were used for antigen capture, and 1 mg beads were used for every 66 pmole of protein. Capture beads were washed 4 times in PBST-B prior to loading with biotinylated antigen. Beads and antigen were allowed to incubate with shaking at 25° C. for 30 minutes. Following incubation, the beads were washed 4 times with PBST-B to remove unbound antigen. Phage library (5×10¹² colony forming units (CFU)) was then applied to the antigen-loaded beads and allowed to incubate with shaking for 1 hour. Each library was panned independently. Following binding, beads were immobilized with a magnet, and the supernatant containing unbound phage was removed. Beads were washed several times for the first round of panning and increasing numbers of washes were performed for later rounds of panning. Following the final wash of the beads, 1 mL of 10 mg/mL trypsin was added and allowed to incubate with shaking for 45 minutes at 37° C. The trypsin solution containing phage was then applied to 10 mL of log-phase TG1 E. co/i in 2xYT media and incubated for 30 minutes without shaking followed by 30 minutes with shaking at 37° C. To calculate the output from the respective round of panning, 10 μL of infected E. coli were collected and serially diluted to calculate the number of CFU recovered. The remaining infected E. coli were pelleted by centrifugation at 6,000×g for 10 minutes at 4° C. The pellet was resuspended in 1 mL 2xTY and plated on 2xYT agar plates supplemented with 100 μg/mL carbenicillin and 2% (w/v) glucose. Plates were incubated overnight at 30° C. For subsequent rounds of panning, depletions on diluted phage were performed by incubation of diluted phage on DYNABEADS™ M-280 Streptavidin prior to exposure to antigen loaded beads. Increasing numbers of washes and wash duration, as well as decreasing antigen concentration, was used to increase stringency across the panning campaign.

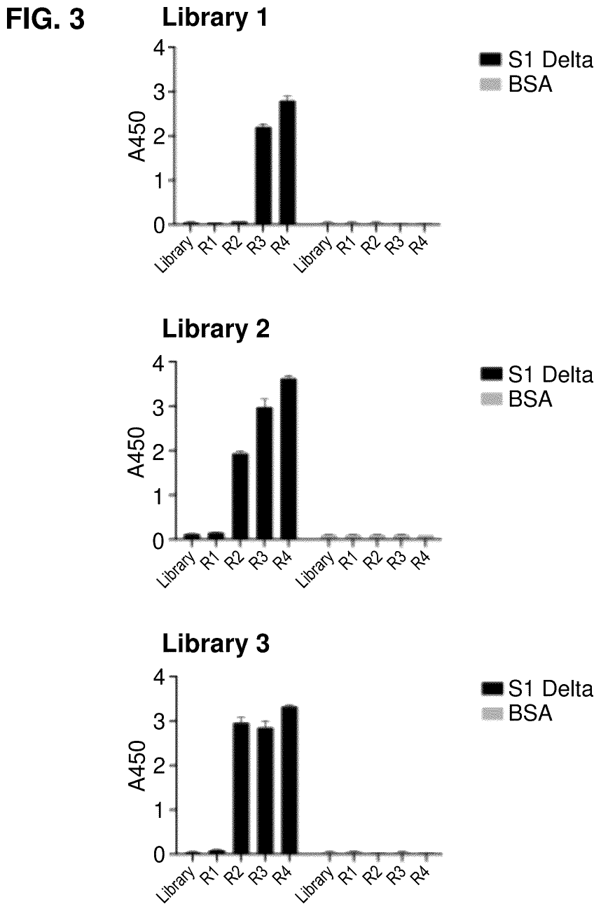

Polyclonal phage ELISA was used to access phage outputs for target specific binding across the whole panning campaign for each individual library. NUNC™ MAXISORP™ 384-well plates were coated with 0.05 μg SARS-CoV-1 S1 protein or BSA for background measurements in 50 mM sodium carbonate pH 9.5 overnight at 4° C. Plates were washed 3 times with PBS supplemented with TWEEN®-20 (PBS-T) and blocked with PBST-MB at 25° C. for 2 hours with shaking. Plates were then washed 3 times with PBS-T, and 1×10⁹ CFU purified phage diluted in PBST-MB was applied to the well and allowed to incubate at 25° C. for 2 hours with shaking. Plates were then washed 3 times with PBS-T, and 1:5000 mouse anti-M13 g8p antibody HRP (Antibody Design Laboratories, AS003-100) diluted in PBST-MB was applied to the well for 1 hour with shaking. Plates were then washed 6 times with PBS-T, and TMB substrate was added to develop the ELISA. Stop solution was added to the well and absorbance at 450 nm was recorded on a Tecan Spark plate reader (FIG. 3).

### Example 6. ELISA Screening of Individual Phage Clones

ELISAs were performed on individual phage clones as follows. For each library tested, E. coli glycerol stocks from Round 3 of panning were plated on 2xYT agar plates supplemented with 100 μg/mL carbenicillin and 2% (w/v) glucose. Individual colonies were picked using a QPIX® 420 colony picking system (Molecular Devices) and were re-arrayed into a 384-well plate with 2xYT media supplemented with 100 μg/mL carbenicillin and 2% (w/v) glucose. Plates were covered with breathable seals and grown overnight at 30° C. Culture media was then exchanged with 2xYT media supplemented with 100 μg/mL carbenicillin and 2.2×10⁷ pfu M13K07 helper phage. These cultures were grown overnight at 30° C., after which cultures were pelleted by centrifugation at 3,500×g for 15 minutes at 4° C. Supernatant from the above was used for clonal ELISA performed below.

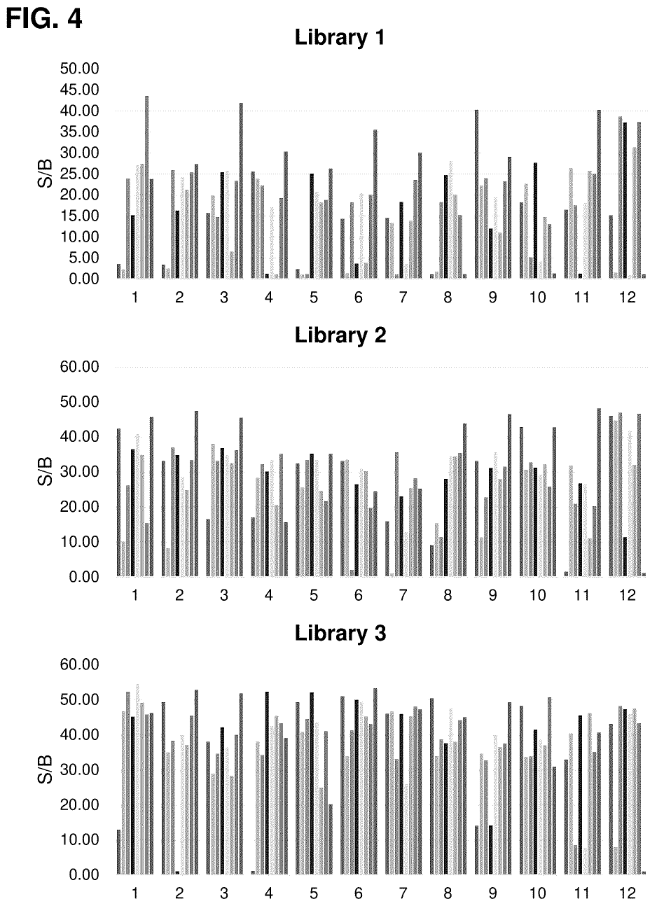

NUNC™ MAXISORP™ 384-well plates were coated with 0.05 μg SARS-CoV-1 S1 protein or BSA for background measurements in 50 mM sodium carbonate pH 9.5 overnight at 4° C. Plates were washed 3 times with PBS supplemented with TWEEN®-20 (PBS-T) and blocked with PBST-MB at 25° C. for 2 hours with shaking. Plates were then washed 3 times with PBS-T, and phage containing supernatant diluted in PBST-MB was applied to the well and allowed to incubate at 25° C. for 2 hours with shaking. Plates were then washed 3 times with PBS-T, and 1:5000 mouse anti-M13 g8p antibody HRP (Antibody Design Laboratories) diluted in PBST-MB was applied to the well for 1 hour with shaking. Plates were then washed 6 times with PBS-T, and TMB substrate was added to develop the ELISA. Stop solution was added to the well and absorbance at 450 nm was recorded on a Tecan Spark plate reader (FIG. 4).

Clones exhibiting an S/B ratio (see Equation 1) of greater than 3 were considered positive hits. To assess sequence diversity, 90 clones were analyzed by Sanger sequencing with a forward and reverse primer covering the whole coding region of the scFv (Table 16). The libraries demonstrated high productivity for the generation of antibodies against canine targets.

TABLE 16
Results of clonal ELISA screening
and Sanger sequencing of hit clones

| | | No. of | S/B | | Unique |
|---|---|---|---|---|---|
| | | Clones | SARS-CoV-2 | Unique | Clonal Types |
| Library | Round | Screened | S1 > 3 | Sequences | (HCDR3) |
| 1 | 3 | 95 | 79 | 14 | 9 |
| 2 | 3 | 95 | 92 | 23 | 23 |
| 3 | 3 | 95 | 93 | 11 | 8 |

| | | | | |
|---|---|---|---|---|
| Total | 285 | 264 | 48 | 40 |

1. A polypeptide library comprising at least 10³ unique polypeptide members, wherein each polypeptide member in the library comprises:
(a) a heavy chain variable domain (VH) comprising VH framework region (FW) 1-heavy chain complementarity determining region (HCDR) 1-VH FW2-HCDR2-VH FW3-HCDR3-VH FW4 segments, wherein the VH FW1 segment comprises the amino acid sequence of SEQ ID NO: 1, the VH FW2 segment comprises the amino acid sequence of SEQ ID NO: 2, the VH FW3 segment comprises the amino acid sequence of SEQ ID NO: 3, and the VH FW4 segment comprises the amino acid sequence of SEQ ID NO: 4; and
(b) a light chain variable domain (VL) comprising VL FW1-light chain complementarity determining region (LCDR) 1-VL FW2-LCDR2-VL FW3-LCDR3-VL FW4 segments, wherein the VL FW1 segment comprises the amino acid sequence of SEQ ID NO: 5, the VL FW2 segment comprises the amino acid sequence of SEQ ID NO: 6, the VL FW3 segment comprises the amino acid sequence of SEQ ID NO: 7, and the VL FW4 segment comprises the amino acid sequence of SEQ ID NO: 8.

2. The polypeptide library of claim 1, wherein:
(a) the HCDR1 segment comprises 10 amino acid residues;
(b) the HCDR2 segment comprises 14 amino acid residues;
(c) the HCDR3 segment comprises from 4 to 16 amino acid residues;
(d) the LCDR1 segment comprises from 4 to 12 amino acid residues;
(e) the LCDR2 segment comprises 8 amino acid residues; and
(f) the LCDR3 segment comprises from 9 to 12 amino acid residues.

3. The polypeptide library of claim 1, wherein:
(a) the HCDR1 segment comprises an amino acid sequence that is at least 80% identical to a sequence selected from the group consisting of SEQ ID NOS: 18-37;
(b) the HCDR2 segment comprises an amino acid sequence that is at least 80% identical to a sequence selected from the group consisting of SEQ ID NOS: 58-77;
(c) the HCDR3 segment comprises an amino acid sequence that is at least 80% identical to a sequence selected from the group consisting of SEQ ID NOS: 98-117;
(d) the LCDR1 segment comprises an amino acid sequence that is at least 80% identical to a sequence selected from the group consisting of SEQ ID NOS: 138-157;
(e) the LCDR2 segment comprises an amino acid sequence that is at least 80% identical to a sequence selected from the group consisting of SEQ ID NOS: 198-217; and
(f) the LCDR3 segment comprises an amino acid sequence that is at least 80% identical to a sequence selected from the group consisting of SEQ ID NOS: 258-277.

4. The polypeptide library of claim 3, wherein:
(a) the HCDR1 segment comprises an amino acid sequence selected from the group consisting of SEQ ID NOS: 18-37;
(b) the HCDR2 segment comprises an amino acid sequence selected from the group consisting of SEQ ID NOS: 58-77;
(c) the HCDR3 segment comprises an amino acid sequence selected from the group consisting of SEQ ID NOS: 98-117;
(d) the LCDR1 segment comprises an amino acid sequence selected from the group consisting of SEQ ID NOS: 138-157;
(e) the LCDR2 segment comprises an amino acid sequence selected from the group consisting of SEQ ID NOS: 198-217; and
(f) the LCDR3 segment comprises an amino acid sequence selected from the group consisting of SEQ ID NOS: 258-277.

5. A polypeptide library comprising at least 10³ unique polypeptide members, wherein each polypeptide member in the library comprises:
(a) a heavy chain variable domain (VH) comprising VH framework region (FW) 1-heavy chain complementarity determining region (HCDR) 1-VH FW2-HCDR2-VH FW3-HCDR3-VH FW4 segments, wherein the VH FW1 segment comprises the amino acid sequence of SEQ ID NO: 1, the VH FW2 segment comprises the amino acid sequence of SEQ ID NO: 2, the VH FW3 segment comprises the amino acid sequence of SEQ ID NO: 3, and the VH FW4 segment comprises the amino acid sequence of SEQ ID NO: 4; and
(b) a light chain variable domain (VL) comprising VL FW1-light chain complementarity determining region (LCDR) 1-VL FW2-LCDR2-VL FW3-LCDR3-VL FW4 segments, wherein the VL FW1 segment comprises the amino acid sequence of SEQ ID NO: 9, the VL FW2 segment comprises the amino acid sequence of SEQ ID NO: 10, the VL FW3 segment comprises the amino acid sequence of SEQ ID NO: 11, and the VL FW4 segment comprises the amino acid sequence of SEQ ID NO: 12.

6. The polypeptide library of claim 5, wherein:
(a) the HCDR1 segment comprises 10 amino acid residues;
(b) the HCDR2 segment comprises 14 amino acid residues;
(c) the HCDR3 segment comprises from 4 to 16 amino acid residues;
(d) the LCDR1 segment comprises from 4 to 18 amino acid residues;
(e) the LCDR2 segment comprises 8 or 12 amino acid residues; and
(f) the LCDR3 segment comprises from 9 to 13 amino acid residues.

7. The polypeptide library of claim 5, wherein:
(a) the HCDR1 segment comprises an amino acid sequence that is at least 80% identical to a sequence selected from the group consisting of SEQ ID NOS: 18-37;
(b) the HCDR2 segment comprises an amino acid sequence that is at least 80% identical to a sequence selected from the group consisting of SEQ ID NOS: 58-77;
(c) the HCDR3 segment comprises an amino acid sequence that is at least 80% identical to a sequence selected from the group consisting of SEQ ID NOS: 98-117;
(d) the LCDR1 segment comprises an amino acid sequence that is at least 80% identical to a sequence selected from the group consisting of SEQ ID NOS: 158-177;
(e) the LCDR2 segment comprises an amino acid sequence that is at least 80% identical to a sequence selected from the group consisting of SEQ ID NOS: 218-237; and
(f) the LCDR3 segment comprises an amino acid sequence that is at least 80% identical to a sequence selected from the group consisting of SEQ ID NOS: 278-297.

8. The polypeptide library of claim 7, wherein:
(a) the HCDR1 segment comprises an amino acid sequence selected from the group consisting of SEQ ID NOS: 18-37;
(b) the HCDR2 segment comprises an amino acid sequence selected from the group consisting of SEQ ID NOS: 58-77;
(c) the HCDR3 segment comprises an amino acid sequence selected from the group consisting of SEQ ID NOS: 98-117;
(d) the LCDR1 segment comprises an amino acid sequence selected from the group consisting of SEQ ID NOS: 158-177;
(e) the LCDR2 segment comprises an amino acid sequence selected from the group consisting of SEQ ID NOS: 218-237; and
(f) the LCDR3 segment comprises an amino acid sequence selected from the group consisting of SEQ ID NOS: 278-297.

9. A polypeptide library comprising at least 10³ unique polypeptide members, wherein each polypeptide member in the library comprises:
(a) a heavy chain variable domain (VH) comprising VH framework region (FW) 1-heavy chain complementarity determining region (HCDR) 1-VH FW2-HCDR2-VH FW3-HCDR3-VH FW4 segments, wherein the VH FW1 segment comprises the amino acid sequence of SEQ ID NO: 13, the VH FW2 segment comprises the amino acid sequence of SEQ ID NO: 14, the VH FW3 segment comprises the amino acid sequence of SEQ ID NO: 15, and the VH FW4 segment comprises the amino acid sequence of SEQ ID NO: 16; and
(b) a light chain variable domain (VL) comprising VL FW1-light chain complementarity determining region (LCDR) 1-VL FW2-LCDR2-VL FW3-LCDR3-VL FW4 segments, wherein the VL FW1 segment comprises the amino acid sequence of SEQ ID NO: 17, the VL FW2 segment comprises the amino acid sequence of SEQ ID NO: 18, the VL FW3 segment comprises the amino acid sequence of SEQ ID NO: 19, and the VL FW4 segment comprises the amino acid sequence of SEQ ID NO: 20.

10. The polypeptide library of claim 9, wherein:
(a) the HCDR1 segment comprises from 8 to 14 amino acid residues;
(b) the HCDR2 segment comprises from 9 to 21 amino acid residues;
(c) the HCDR3 segment comprises from 2 to 29 amino acid residues;
(d) the LCDR1 segment comprises from 5 to 16 amino acid residues;
(e) the LCDR2 segment comprises 7 or 8 amino acid residues; and
(f) the LCDR3 segment comprises from 5 to 16 amino acid residues.

11. The polypeptide library of claim 9, wherein:
(a) the HCDR1 segment comprises an amino acid sequence that is at least 80% identical to a sequence selected from the group consisting of SEQ ID NOS: 38-57;
(b) the HCDR2 segment comprises an amino acid sequence that is at least 80% identical to a sequence selected from the group consisting of SEQ ID NOS: 78-97;
(c) the HCDR3 segment comprises an amino acid sequence that is at least 80% identical to a sequence selected from the group consisting of SEQ ID NOS: 118-137;
(d) the LCDR1 segment comprises an amino acid sequence that is at least 80% identical to a sequence selected from the group consisting of SEQ ID NOS: 178-197;
(e) the LCDR2 segment comprises an amino acid sequence that is at least 80% identical to a sequence selected from the group consisting of SEQ ID NOS: 238-257; and
(f) the LCDR3 segment comprises an amino acid sequence that is at least 80% identical to a sequence selected from the group consisting of SEQ ID NOS: 298-317.

12. The polypeptide library of claim 11, wherein:
(a) the HCDR1 segment comprises an amino acid sequence selected from the group consisting of SEQ ID NOS: 38-57;
(b) the HCDR2 segment comprises an amino acid sequence selected from the group consisting of SEQ ID NOS: 78-97;
(c) the HCDR3 segment comprises an amino acid sequence selected from the group consisting of SEQ ID NOS: 118-137;
(d) the LCDR1 segment comprises an amino acid sequence selected from the group consisting of SEQ ID NOS: 178-197;
(e) the LCDR2 segment comprises an amino acid sequence selected from the group consisting of SEQ ID NOS: 238-257; and
(f) the LCDR3 segment comprises an amino acid sequence selected from the group consisting of SEQ ID NOS: 298-317.

13. The polypeptide library of claim 1, wherein the polypeptide library comprising at least 10⁵ or at least 10⁶ unique polypeptide members.

14. (canceled)

15. The polypeptide library of claim 1, wherein the polypeptide library comprises one or more antibodies or antigen-binding fragments thereof.

16. The polypeptide library of claim 15, wherein the antibody or antigen-binding fragment thereof is selected from the group consisting of a monoclonal antibody or antigen-binding fragment thereof, a polyclonal antibody or antigen-binding fragment thereof, a canine antibody or antigen-binding fragment thereof, a caninized antibody or antigen-binding fragment thereof, a bispecific antibody or antigen-binding fragment thereof, a multi-specific antibody or antigen-binding fragment thereof, a dual-variable immunoglobulin domain, a monovalent antibody or antigen-binding fragment thereof, a chimeric antibody or antigen-binding fragment thereof, a single-chain Fv molecule (scFv), a diabody, a triabody, a Fv fragment, a Fab fragment, a F(ab′)2 molecule, and a tandem scFv (taFv).

17. A collection of nucleic acids encoding the polypeptide library of claim 1.

18. A collection of vectors comprising the collection of nucleic acids of claim 17.

19. A collection of host cells comprising the collection of nucleic acids of claim 17, a collection of vectors comprising the collection of nucleic acids of claim 17.

20. A method of isolating an antibody or antigen-binding fragment thereof that specifically binds to an antigen, the method comprising the steps of:
(a) contacting the polypeptide library of claim 1 with the antigen;
(b) removing the members of the polypeptide library that do not bind to the antigen; and
(c) recovering the members of the polypeptide library that bind to the antigen.

21. An antibody or antigen-binding fragment thereof, wherein:
(a) the antibody or antigen-binding fragment thereof is isolated from the polypeptide library of claim 1; or
(b) the antibody or antigen-binding fragment thereof is isolated by a method comprising the steps of:
(i) contacting the polypeptide library of claim 1 with the antigen;
(ii) removing the members of the polypeptide library that do not bind to the antigen; and
(iii) recovering the members of the polypeptide library that bind to the antigen.
